(19) United States
(12) Patent Application Publication
(10) Pub. No.: US 2016/0017041 A1
(43) Pub. Date: Jan. 21, 2016

(54) TREATMENT AND PREVENTION OF ACUTE KIDNEY INJURY USING ANTI-ALPHA V BETA 5 ANTIBODIES

(71) Applicant: BIOGEN MA INC., Cambridge, MA (US)

(72) Inventors: Shelia M. Violette, Lexington, MA (US); Bradley J. Maroni, Boston, MA (US)

(21) Appl. No.: 14/770,522

(22) PCT Filed: Mar. 13, 2014
(86) PCT No.: PCT/US2014/026234

§ 371 Date: Aug. 26, 2015

Related U.S. Application Data
(60) Provisional application No. 61/792,681, filed on Mar. 15, 2013.
(60) Provisional application No. 61/898,811, filed on Nov. 1, 2013.

(51) Int. Cl. C07K 16/28; A61K 45/06; A61K 39/395

(52) U.S. Cl. CPC C07K 16/2839; A61K 39/3955; A61K 45/06; A61K 2039/505

(57) ABSTRACT

Methods of using antibodies and antibody fragments that specifically bind αvβ5 or β5 to treat or prevent acute kidney injury and/or its sequelae are disclosed.

### CROSS-REFERENCE TO RELATED APPLICATIONS

This application claims the benefit of priority to U.S. Provisional Appl. No. 61/792,681 filed Mar. 15, 2013, and U.S. Provisional Appl. No. 61/898,811 filed Nov. 1, 2013, the contents of both of which are incorporated by reference in their entirety herein.

### FIELD

This invention relates generally to the use of antibodies or antigen-binding fragments thereof that bind to the alpha v beta 5 (αvβ₅) integrin for the treatment or prevention of acute kidney injury.

### BACKGROUND

Acute kidney injury (formerly known as acute renal failure) is a severe inflammation and damage of the kidney, which sometimes results in complete kidney failure. Acute kidney injury is characterized by the rapid loss of the kidney's excretory function and is typically diagnosed by the accumulation of end products of nitrogen metabolism (urea and creatinine) or decreased urine output, or both. It is the clinical manifestation of several disorders that affect the kidney acutely. Patients who have had acute kidney injury are at increased risk of developing chronic kidney disease.

Acute kidney injury is a condition that is common in hospital patients and very common in critically ill patients. Hospital-acquired acute kidney injury affects approximately 2 million patients in the Western World. Thus, it poses a significant clinical problem that complicates the course of hospitalization and portends worse clinical outcomes for hospitalized patients.

Acute kidney injury diagnoses are increasing in part because of an aging population, increased exposure to nephrotoxic drugs or infections in hospitals, as well as an increasing number of surgical interventions. Depending on the severity of kidney failure, the mortality rate ranges from 7% to as high as 80%, with an average of approximately 35%. Approximately 700,000 deaths in Europe, the US, and Japan each year are linked to this disease.

Accordingly, there is a great need for therapeutic agents that can treat or prevent acute kidney injury and its subsequent complications.

### SUMMARY

This disclosure features methods of using antibodies and antigen-binding fragments thereof that specifically bind to αvβ5 and/or β5 and their use in treating, preventing, or reducing the symptoms or severity of acute kidney injury.

In one aspect, the application discloses a method for treating, preventing, or reducing the severity of acute kidney injury or a complication thereof in a human subject in need thereof. The method involves administering to the human subject an effective amount of an antibody or an antigen-binding fragment thereof that specifically binds to the αvβ5 integrin. In certain embodiments, the antibody or the antigen-binding fragment thereof specifically binds to the β5 subunit of the αvβ5 integrin. In certain embodiments, the antibody or the antigen-binding fragment thereof is an αvβ5 antagonist.

In some embodiments, the antibody or the antigen-binding fragment thereof competes with an antibody produced by the hybridoma deposited as ATCC Deposit No. PTA-5817. In some embodiments, the antibody competes with an antibody produced by the hybridoma deposited as ATCC Deposit No. PTA-5817, wherein the effector function of the antibody is reduced or eliminated. In other embodiments, the antibody or the antigen-binding fragment thereof binds the same epitope as an antibody produced by the hybridoma deposited as ATCC Deposit No. PTA-5817. In certain embodiments, the antibody binds the same epitope as an antibody produced by the hybridoma deposited as ATCC Deposit No. PTA-5817, wherein the effector function of the antibody is reduced or eliminated. In other embodiments, the antibody or the antigen-binding fragment thereof comprises the heavy chain variable region CDR1, CDR2, and CDR3 of the antibody produced by the hybridoma deposited as ATCC Deposit No. PTA-5817. In certain embodiments, the antibody or the antigen-binding fragment thereof further comprises the light chain variable region CDR1, CDR2, and CDR3 of the antibody produced by the hybridoma deposited as ATCC Deposit No. PTA-5817. In other embodiments, the antibody or the antigen-binding fragment thereof is a humanized form of the antibody produced by the hybridoma deposited as ATCC Deposit No. PTA-5817. In other embodiments, the antibody or the antigen-binding fragment thereof is a humanized form of the antibody produced by the hybridoma deposited as ATCC Deposit No. PTA-5817, wherein the effector function of the antibody is reduced or eliminated.

In certain embodiments, the antibody or the antigen-binding fragment thereof comprises a VH CDR1 comprising or consisting of the amino acid sequence set forth in SEQ ID NO:3 or SEQ ID NO:3 with two or fewer substitutions (i.e., 2, 1, or 0 substitutions); a VH CDR2 comprising or consisting of the amino acid sequence set forth in SEQ ID NO:4 or SEQ ID NO:4 with two or fewer substitutions; and a VH CDR3 comprising or consisting of the amino acid sequence set forth in SEQ ID NO:5 or SEQ ID NO:5 with two or fewer substitutions. In certain embodiments, the antibody or the antigen-binding fragment thereof comprises a VH CDR1 comprising or consisting of the amino acid sequence set forth in SEQ ID NO:21 or SEQ ID NO:21 with two or fewer substitutions; a VH CDR2 comprising or consisting of the amino acid sequence set forth in SEQ ID NO:24 or SEQ ID NO:24 with two or fewer substitutions; and a VH CDR3 comprising or consisting of the amino acid sequence set forth in SEQ ID NO:5 or SEQ ID NO:5 with two or fewer substitutions. In certain embodiments, the antibody or the antigen-binding fragment thereof comprises a VH CDR1 comprising or consisting of the amino acid sequence set forth in SEQ ID NO:22 or SEQ ID NO:22 with two or fewer substitutions; a VH CDR2 comprising or consisting of the amino acid sequence set forth in SEQ ID NO:25 or SEQ ID NO:25 with two or fewer substitutions; and a VH CDR3 comprising or consisting of the amino acid sequence set forth in SEQ ID NO:5 or SEQ ID NO:5 with two or fewer substitutions. In certain embodiments, the antibody or the antigen-binding fragment thereof comprises a VH CDR1 comprising or consisting of the amino acid sequence set forth in SEQ ID NO:23 or SEQ ID NO:23 with two or fewer substitutions; a VH CDR2 comprising or consisting of the amino acid sequence set forth in SEQ ID NO:26 or SEQ ID NO:26 with two or fewer substitutions; and a VH CDR3 comprising or consisting of the amino acid sequence set forth in SEQ ID NO:27 or SEQ ID NO:27 with two or fewer substitutions. In certain embodiments, the antibody or the antigen-binding fragment thereof comprises a VH CDR1 comprising or consisting of the amino acid sequence set forth in SEQ ID NO:3 or SEQ ID NO:3 with one substitution; a VH CDR2 comprising or consisting of the amino acid sequence set forth in SEQ ID NO:4 or SEQ ID NO:4 with one substitution; and a VH CDR3 comprising or consisting of the amino acid sequence set forth in SEQ ID NO:5 or SEQ ID NO:5 with one substitution. In certain embodiments, the antibody or the antigen-binding fragment thereof comprises a VH CDR1 comprising or consisting of the amino acid sequence set forth in SEQ ID NO:21 or SEQ ID NO:21 with one substitution; a VH CDR2 comprising or consisting of the amino acid sequence set forth in SEQ ID NO:24 or SEQ ID NO:24 with one substitution; and a VH CDR3 comprising or consisting of the amino acid sequence set forth in SEQ ID NO:5 or SEQ ID NO:5 with one substitution. In certain embodiments, the antibody or the antigen-binding fragment thereof comprises a VH CDR1 comprising or consisting of the amino acid sequence set forth in SEQ ID NO:22 or SEQ ID NO:22 with one substitution; a VH CDR2 comprising or consisting of the amino acid sequence set forth in SEQ ID NO:25 or SEQ ID NO:25 with one substitution; and a VH CDR3 comprising or consisting of the amino acid sequence set forth in SEQ ID NO:5 or SEQ ID NO:5 with one substitution. In certain embodiments, the antibody or the antigen-binding fragment thereof comprises a VH CDR1 comprising or consisting of the amino acid sequence set forth in SEQ ID NO:23 or SEQ ID NO:23 with one substitution; a VH CDR2 comprising or consisting of the amino acid sequence set forth in SEQ ID NO:26 or SEQ ID NO:26 with one substitution; and a VH CDR3 comprising or consisting of the amino acid sequence set forth in SEQ ID NO:27 or SEQ ID NO:27 with one substitution. In certain embodiments, the antibody or the antigen-binding fragment thereof comprises a VH CDR1 comprising or consisting of the amino acid sequence set forth in SEQ ID NO:3; a VH CDR2 comprising or consisting of the amino acid sequence set forth in SEQ ID NO:4; and a VH CDR3 comprising or consisting of the amino acid sequence set forth in SEQ ID NO:5. In certain embodiments, the antibody or the antigen-binding fragment thereof comprises a VH CDR1 comprising or consisting of the amino acid sequence set forth in SEQ ID NO:21; a VH CDR2 comprising or consisting of the amino acid sequence set forth in SEQ ID NO:24; and a VH CDR3 comprising or consisting of the amino acid sequence set forth in SEQ ID NO:5. In certain embodiments, the antibody or the antigen-binding fragment thereof comprises a VH CDR1 comprising or consisting of the amino acid sequence set forth in SEQ ID NO:22; a VH CDR2 comprising or consisting of the amino acid sequence set forth in SEQ ID NO:25; and a VH CDR3 comprising or consisting of the amino acid sequence set forth in SEQ ID NO:5. In certain embodiments, the antibody or the antigen-binding fragment thereof comprises a VH CDR1 comprising or consisting of the amino acid sequence set forth in SEQ ID NO:23; a VH CDR2 comprising or consisting of the amino acid sequence set forth in SEQ ID NO:26; and a VH CDR3 comprising or consisting of the amino acid sequence set forth in SEQ ID NO:27.

In certain embodiments, the antibody or the antigen-binding fragment thereof in addition to comprising the VH CDRs 1, 2, and 3 described in the paragraph above, further comprises a VL CDR1 comprising or consisting of the amino acid sequence set forth in SEQ ID NO:6 or SEQ ID NO:6 with two or fewer substitutions; a VL CDR2 comprising or consisting of the amino acid sequence set forth in SEQ ID NO:7 or SEQ ID NO:7 with two or fewer substitutions; and a VL CDR3 comprising or consisting of the amino acid sequence set forth in SEQ ID NO:8 or SEQ ID NO:8 with two or fewer substitutions. In certain other embodiments, the antibody or the antigen-binding fragment thereof in addition to comprising the VH CDRs1, 2, and 3 described in the paragraph above, further comprises a VL CDR1 comprising or consisting of the amino acid sequence set forth in SEQ ID NO:28 or SEQ ID NO:28 with two or fewer substitutions; a VL CDR2 comprising or consisting of the amino acid sequence set forth in SEQ ID NO:29 or SEQ ID NO:29 with two or fewer substitutions; and a VL CDR3 comprising or consisting of the amino acid sequence set forth in SEQ ID NO:30 or SEQ ID NO:30 with two or fewer substitutions.

In certain embodiments, the antibody or the antigen-binding fragment thereof is a humanized antibody comprising a VH CDR1 comprising or consisting of the amino acid sequence set forth in SEQ ID NO:3 or SEQ ID NO:3 with two or fewer substitutions; a VH CDR2 comprising or consisting of the amino acid sequence set forth in SEQ ID NO:4 or SEQ ID NO:4 with two or fewer substitutions; a VH CDR3 comprising or consisting of the amino acid sequence set forth in SEQ ID NO:5 or SEQ ID NO:5 with two or fewer substitutions; a VL CDR1 comprising or consisting of the amino acid sequence set forth in SEQ ID NO:6 or SEQ ID NO:6 with two or fewer substitutions; a VL CDR2 comprising or consisting of the amino acid sequence set forth in SEQ ID NO:7 or SEQ ID NO:7 with two or fewer substitutions; and a VL CDR3 comprising or consisting of the amino acid sequence set forth in SEQ ID NO:8 or SEQ ID NO:8 with two or fewer substitutions, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:21 or SEQ ID NO:21 with two or fewer substitutions; a VH CDR2 comprising or consisting of the amino acid sequence set forth in SEQ ID NO:24 or SEQ ID NO:24 with two or fewer substitutions; a VH CDR3 comprising or consisting of the amino acid sequence set forth in SEQ ID NO:5 or SEQ ID NO:5 with two or fewer substitutions; a VL CDR1 comprising or consisting of the amino acid sequence set forth in SEQ ID NO:6 or SEQ ID NO:6 with two or fewer substitutions; a VL CDR2 comprising or consisting of the amino acid sequence set forth in SEQ ID NO:7 or SEQ ID NO:7 with two or fewer substitutions; and a VL CDR3 comprising or consisting of the amino acid sequence set forth in SEQ ID NO:8 or SEQ ID NO:8 with two or fewer substitutions, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:22 or SEQ ID NO:22 with two or fewer substitutions; a VH CDR2 comprising or consisting of the amino acid sequence set forth in SEQ ID NO:25 or SEQ ID NO:25 with two or fewer substitutions; a VH CDR3 comprising or consisting of the amino acid sequence set forth in SEQ ID NO:5 or SEQ ID NO:5 with two or fewer substitutions; a VL CDR1 comprising or consisting of the amino acid sequence set forth in SEQ ID NO:6 or SEQ ID NO:6 with two or fewer substitutions; a VL CDR2 comprising or consisting of the amino acid sequence set forth in SEQ ID NO:7 or SEQ ID NO:7 with two or fewer substitutions; and a VL CDR3 comprising or consisting of the amino acid sequence set forth in SEQ ID NO:8 or SEQ ID NO:8 with two or fewer substitutions, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:23 or SEQ ID NO:23 with two or fewer substitutions; a VH CDR2 comprising or consisting of the amino acid sequence set forth in SEQ ID NO:26 or SEQ ID NO:26 with two or fewer substitutions; a VH CDR3 comprising or consisting of the amino acid sequence set forth in SEQ ID NO:27 or SEQ ID NO:27 with two or fewer substitutions; a VL CDR1 comprising or consisting of the amino acid sequence set forth in SEQ ID NO:28 or SEQ ID NO:28 with two or fewer substitutions; a VL CDR2 comprising or consisting of the amino acid sequence set forth in SEQ ID NO:29 or SEQ ID NO:29 with two or fewer substitutions; and a VL CDR3 comprising or consisting of the amino acid sequence set forth in SEQ ID NO:30 or SEQ ID NO:30 with two or fewer substitutions, wherein the effector function of the humanized antibody is reduced or eliminated.

In certain embodiments, the antibody or the antigen-binding fragment thereof is a humanized antibody comprising a VH CDR1 comprising or consisting of the amino acid sequence set forth in SEQ ID NO:3 or SEQ ID NO:3 with one substitution; a VH CDR2 comprising or consisting of the amino acid sequence set forth in SEQ ID NO:4 or SEQ ID NO:4 with one substitution; a VH CDR3 comprising or consisting of the amino acid sequence set forth in SEQ ID NO:5 or SEQ ID NO:5 with one substitution; a VL CDR1 comprising or consisting of the amino acid sequence set forth in SEQ ID NO:6 or SEQ ID NO:6 with one substitution; a VL CDR2 comprising or consisting of the amino acid sequence set forth in SEQ ID NO:7 or SEQ ID NO:7 with one substitution; and a VL CDR3 comprising or consisting of the amino acid sequence set forth in SEQ ID NO:8 or SEQ ID NO:8 with one substitution, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:21 or SEQ ID NO:21 with one substitution; a VH CDR2 comprising or consisting of the amino acid sequence set forth in SEQ ID NO:24 or SEQ ID NO:24 with one substitution; a VH CDR3 comprising or consisting of the amino acid sequence set forth in SEQ ID NO:5 or SEQ ID NO:5 with one substitution; a VL CDR1 comprising or consisting of the amino acid sequence set forth in SEQ ID NO:6 or SEQ ID NO:6 with one substitution; a VL CDR2 comprising or consisting of the amino acid sequence set forth in SEQ ID NO:7 or SEQ ID NO:7 with one substitution; and a VL CDR3 comprising or consisting of the amino acid sequence set forth in SEQ ID NO:8 or SEQ ID NO:8 with one substitution, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:22 or SEQ ID NO:22 with one substitution; a VH CDR2 comprising or consisting of the amino acid sequence set forth in SEQ ID NO:25 or SEQ ID NO:25 with one substitution; a VH CDR3 comprising or consisting of the amino acid sequence set forth in SEQ ID NO:5 or SEQ ID NO:5 with one substitution; a VL CDR1 comprising or consisting of the amino acid sequence set forth in SEQ ID NO:6 or SEQ ID NO:6 with one substitution; a VL CDR2 comprising or consisting of the amino acid sequence set forth in SEQ ID NO:7 or SEQ ID NO:7 with one substitution; and a VL CDR3 comprising or consisting of the amino acid sequence set forth in SEQ ID NO:8 or SEQ ID NO:8 with one substitution, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:23 or SEQ ID NO:23 with one substitution; a VH CDR2 comprising or consisting of the amino acid sequence set forth in SEQ ID NO:26 or SEQ ID NO:26 with one substitution; a VH CDR3 comprising or consisting of the amino acid sequence set forth in SEQ ID NO:27 or SEQ ID NO:27 with one substitution; a VL CDR1 comprising or consisting of the amino acid sequence set forth in SEQ ID NO:28 or SEQ ID NO:28 with one substitution; a VL CDR2 comprising or consisting of the amino acid sequence set forth in SEQ ID NO:29 or SEQ ID NO:29 with one substitution; and a VL CDR3 comprising or consisting of the amino acid sequence set forth in SEQ ID NO:30 or SEQ ID NO:30 with one substitution, wherein the effector function of the humanized antibody is reduced or eliminated.

In other embodiments, the antibody or the antigen-binding fragment thereof is a humanized antibody comprising a VH CDR1 comprising or consisting of the amino acid sequence set forth in SEQ ID NO:3; a VH CDR2 comprising or consisting of the amino acid sequence set forth in SEQ ID NO:4; a VH CDR3 comprising or consisting of the amino acid sequence set forth in SEQ ID NO:5; a VL CDR1 comprising or consisting of the amino acid sequence set forth in SEQ ID NO:6; a VL CDR2 comprising or consisting of the amino acid sequence set forth in SEQ ID NO:7; and a VL CDR3 comprising or consisting of the amino acid sequence set forth in SEQ ID NO:8, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:21; a VH CDR2 comprising or consisting of the amino acid sequence set forth in SEQ ID NO:24; a VH CDR3 comprising or consisting of the amino acid sequence set forth in SEQ ID NO:5; a VL CDR1 comprising or consisting of the amino acid sequence set forth in SEQ ID NO:6; a VL CDR2 comprising or consisting of the amino acid sequence set forth in SEQ ID NO:7; and a VL CDR3 comprising or consisting of the amino acid sequence set forth in SEQ ID NO:8, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:22; a VH CDR2 comprising or consisting of the amino acid sequence set forth in SEQ ID NO:25; a VH CDR3 comprising or consisting of the amino acid sequence set forth in SEQ ID NO:5; a VL CDR1 comprising or consisting of the amino acid sequence set forth in SEQ ID NO:6; a VL CDR2 comprising or consisting of the amino acid sequence set forth in SEQ ID NO:7; and a VL CDR3 comprising or consisting of the amino acid sequence set forth in SEQ ID NO:8, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:23; a VH CDR2 comprising or consisting of the amino acid sequence set forth in SEQ ID NO:26; a VH CDR3 comprising or consisting of the amino acid sequence set forth in SEQ ID NO:27; a VL CDR1 comprising or consisting of the amino acid sequence set forth in SEQ ID NO:28; a VL CDR2 comprising or consisting of the amino acid sequence set forth in SEQ ID NO:29; and a VL CDR3 comprising or consisting of the amino acid sequence set forth in SEQ ID NO:30, wherein the effector function of the humanized antibody is reduced or eliminated.

In some embodiments, the antibody or the antigen-binding fragment thereof is administered intravenously, subcutaneously, or intraarterially.

In certain embodiments, the human subject has been identified as having acute kidney injury based on the Acute Kidney Injury Network (AKIN) criteria or Risk/Injury/Failure/Loss/ESRD (RIFLE) criteria.

In another embodiment, the human subject has been identified as having an elevated level of serum creatinine, plasma creatinine, urine creatinine, or blood urea nitrogen (BUN), compared to a healthy control subject.

In another embodiment, the human subject has been identified as having an elevated level of serum or urine neutrophil gelatinase-associated lipocalin, serum or urine interleukin-18, serum or urine cystatin C, or urine KIM-1, compared to a healthy control subject.

In some embodiments, the acute kidney injury is an ischemic acute kidney injury. In one embodiment, the human subject has been identified as having reduced effective arterial volume. In one embodiment, the human subject has been identified as having intravascular volume depletion (e.g., due to hemorrhage, gastrointestinal loss, renal loss, skin and mucous membrane loss, nephrotic syndrome, cirrhosis, or capillary leak). In one embodiment, the human subject has been identified as having reduced cardiac output (e.g., due to cardiogenic shock, pericardial disease, congestive heart failure, valvular heart disease, pulmonary disease, or sepsis). In one embodiment, the human subject has been identified as having systemic vasodilation (e.g., caused by cirrhosis, anaphylaxis, or sepsis). In one embodiment, the human subject has been identified as having renal vasoconstriction (e.g., caused by early sepsis, hepatorenal syndrome, acute hypercalcemia, a drug, or a radiocontrast agent).

In some embodiments, the acute kidney injury is a nephrotoxic acute kidney injury. In one embodiment, the human subject has been exposed to a nephrotoxin. For example, the nephrotoxin can be a nephrotoxic drug selected from the group consisting of an antibiotic (e.g., an aminoglycoside), a chemotherapeutic agent (e.g., cis-platinum), a calcineurin inhibitor, amphotericin B, and a radiographic contrast agent. In another example, the nephrotoxin can be an illicit drug or a heavy metal.

In certain embodiments, the human subject has undergone a trauma injury or a crush injury.

In certain embodiments, the human subject has undergone an organ transplant surgery (e.g., a kidney transplant surgery or heart transplant surgery).

In certain embodiments, the human subject has undergone a surgery complicated by hypoperfusion.

In certain embodiments, the human subject has undergone cardiothoracic surgery or a vascular surgery.

In certain embodiments, the human subject has taken medication (e.g., an anticholinergic) that interferes with normal emptying of the bladder.

In certain embodiments, the human subject has benign prostatic hypertrophy or a cancer (e.g., prostate cancer, ovarian cancer, or colorectal cancer).

In certain embodiments, the human subject has a kidney stone.

In certain embodiments, the human subject has an obstructed urinary catheter.

In certain embodiments, the human subject has taken a drug that causes or leads to crystalluria, a drug that causes or leads to myoglobinuria, or a drug that causes or leads to cystitis.

In some embodiments, the human subject is administered a second therapeutic agent selected from the group consisting of an αvβ5 integrin inhibitor, an αvβ6 integrin inhibitor, a CXCR4 antagonist, an IL-6 inhibitor, an IL-1α inhibitor, an IL-12 inhibitor, a MIP-1-α inhibitor, AP214, THR-184, QPI-1002, a human alkaline phosphatase, an anti-apoptosis agent, an anti-necrosis agent, an anti-inflammatory agent, an anti-sepsis agent, a growth factor, a vasodilator, a free radical scavenger, neutrophil gelatinase-associated lipocalin, a C5a receptor antagonist, and α-melanocyte-stimulating hormone.

In a further aspect, the application discloses a method for protecting a kidney from injury in a human subject. The method involves administering to the human subject an effective amount of an antibody or an antigen-binding fragment thereof that specifically binds to the αvβ5 integrin. In certain embodiments, the antibody or the antigen-binding fragment thereof specifically binds to the β5 subunit of the αvβ5 integrin. In certain embodiments, the antibody or the antigen-binding fragment thereof is an αvβ5 antagonist. In certain embodiments, the injury is to the epithelium of the kidney. In certain embodiments, the injury is tubular epithelial injury. In some embodiments, the human subject has been or will be exposed to an ischemic or nephrotoxic insult. In some embodiments, the human subject has been exposed to oxidative damage (e.g., by free radicals such as reactive oxygen or nitrogen species).

In yet another aspect, the application discloses a method for treating a human subject having an injury to the kidney. The method involves administering to the human subject an effective amount of an antibody or an antigen-binding fragment thereof that specifically binds to the αvβ5 integrin. In certain embodiments, the antibody or the antigen-binding fragment thereof specifically binds to the β5 subunit of the αvβ5 integrin. In certain embodiments, the antibody or the antigen-binding fragment thereof is an αvβ5 antagonist. In certain embodiments, the injury is to the epithelium of the kidney. In certain embodiments, the injury is tubular epithelial injury. In some embodiments, the human subject has been or will be exposed to an ischemic or nephrotoxic insult. In some embodiments, the human subject has been exposed to oxidative damage (e.g., by free radicals such as reactive oxygen or nitrogen species).

In another aspect, the application discloses a method for improving urine flow in a human subject. The method involves administering to the human subject an effective amount of an antibody or an antigen-binding fragment thereof that specifically binds to the αvβ5 integrin. In certain embodiments, the antibody or the antigen-binding fragment thereof specifically binds to the β5 subunit of the αvβ5 integrin. In certain embodiments, the antibody or the antigen-binding fragment thereof is an αvβ5 antagonist.

In a further aspect, the application discloses a method for protecting a human subject's kidney from acute kidney injury during transplantation. The method involves administering to the human subject an effective amount of an antibody or an antigen-binding fragment thereof that specifically binds to the αvβ5 integrin prior to or at the same time as the kidney transplantation. In certain embodiments, the antibody or the antigen-binding fragment thereof specifically binds to the β5 subunit of the αvβ5 integrin. In certain embodiments, the antibody or the antigen-binding fragment thereof is an αvβ5 antagonist. In certain embodiments, the method further comprises administering to the human subject one or more doses of an antibody or an antigen-binding fragment thereof that specifically binds to the αvβ5 integrin after (e.g., 0.1, 0.2, 0.3, 0.4, 0.5, 1, 2, 3, 4, 5, 6, 7, 8, 9, 10, 11, 12, 24, 48, 72, 96,168 hours, or lweek, 2 weeks, 3 weeks or 1 month) the kidney transplantation.

In some embodiments of the above aspects, the antibody or the antigen-binding fragment thereof competes with an antibody produced by the hybridoma deposited as ATCC Deposit No. PTA-5817. In some embodiments, the antibody competes with an antibody produced by the hybridoma deposited as ATCC Deposit No. PTA-5817, wherein the effector function of the antibody is reduced or eliminated. In other embodiments, the antibody or the antigen-binding fragment thereof binds the same epitope as an antibody produced by the hybridoma deposited as ATCC Deposit No. PTA-5817. In certain embodiments, the antibody binds the same epitope as an antibody produced by the hybridoma deposited as ATCC Deposit No. PTA-5817, wherein the effector function of the antibody is reduced or eliminated. In other embodiments, the antibody or the antigen-binding fragment thereof comprises the heavy chain variable region CDR1, CDR2, and CDR3 of the antibody produced by the hybridoma deposited as ATCC Deposit No. PTA-5817. In certain embodiments, the antibody or the antigen-binding fragment thereof further comprises the light chain variable region CDR1, CDR2, and CDR3 of the antibody produced by the hybridoma deposited as ATCC Deposit No. PTA-5817. In other embodiments, the antibody or the antigen-binding fragment thereof is a humanized form of the antibody produced by the hybridoma deposited as ATCC Deposit No. PTA-5817. In other embodiments, the antibody or the antigen-binding fragment thereof is a humanized form of the antibody produced by the hybridoma deposited as ATCC Deposit No. PTA-5817, wherein the effector function of the antibody is reduced or eliminated.

In some embodiments of the above aspects, the antibody or the antigen-binding fragment thereof comprises a VH CDR1 having comprising or consisting of the amino acid sequence set forth in SEQ ID NO:3 or SEQ ID NO:3 with two or fewer substitutions (i.e., 2, 1, or 0 substitutions); a VH CDR2 comprising or consisting of having the amino acid sequence set forth in SEQ ID NO:4 or SEQ ID NO:4 with two or fewer substitutions; and a VH CDR3 comprising or consisting of having the amino acid sequence set forth in SEQ ID NO:5 or SEQ ID NO:5 with two or fewer substitutions. In certain embodiments, the antibody or the antigen-binding fragment thereof comprises a VH CDR1 comprising or consisting of having the amino acid sequence set forth in SEQ ID NO:21 or SEQ ID NO:21 with two or fewer substitutions; a VH CDR2 comprising or consisting of having the amino acid sequence set forth in SEQ ID NO:24 or SEQ ID NO:24 with two or fewer substitutions; and a VH CDR3 comprising or consisting of having the amino acid sequence set forth in SEQ ID NO:5 or SEQ ID NO:5 with two or fewer substitutions. In certain embodiments, the antibody or the antigen-binding fragment thereof comprises a VH CDR1 comprising or consisting of having the amino acid sequence set forth in SEQ ID NO:22 or SEQ ID NO:22 with two or fewer substitutions; a VH CDR2 comprising or consisting of having the amino acid sequence set forth in SEQ ID NO:25 or SEQ ID NO:25 with two or fewer substitutions; and a VH CDR3 comprising or consisting of having the amino acid sequence set forth in SEQ ID NO:5 or SEQ ID NO:5 with two or fewer substitutions. In certain embodiments, the antibody or the antigen-binding fragment thereof comprises a VH CDR1 comprising or consisting of the amino acid sequence set forth in SEQ ID NO:23 or SEQ ID NO:23 with two or fewer substitutions; a VH CDR2 comprising or consisting of the amino acid sequence set forth in SEQ ID NO:26 or SEQ ID NO:26 with two or fewer substitutions; and a VH CDR3 comprising or consisting of the amino acid sequence set forth in SEQ ID NO:27 or SEQ ID NO:27 with two or fewer substitutions. In certain embodiments, the antibody or the antigen-binding fragment thereof comprises a VH CDR1 comprising or consisting of the amino acid sequence set forth in SEQ ID NO:3 or SEQ ID NO:3 with one substitution; a VH CDR2 comprising or consisting of the amino acid sequence set forth in SEQ ID NO:4 or SEQ ID NO:4 with one substitution; and a VH CDR3 comprising or consisting of the amino acid sequence set forth in SEQ ID NO:5 or SEQ ID NO:5 with one substitution. In certain embodiments, the antibody or the antigen-binding fragment thereof comprises a VH CDR1 comprising or consisting of the amino acid sequence set forth in SEQ ID NO:21 or SEQ ID NO:21 with one substitution; a VH CDR2 comprising or consisting of the amino acid sequence set forth in SEQ ID NO:24 or SEQ ID NO:24 with one substitution; and a VH CDR3 comprising or consisting of the amino acid sequence set forth in SEQ ID NO:5 or SEQ ID NO:5 with one substitution. In certain embodiments, the antibody or the antigen-binding fragment thereof comprises a VH CDR1 comprising or consisting of the amino acid sequence set forth in SEQ ID NO:22 or SEQ ID NO:22 with one substitution; a VH CDR2 comprising or consisting of the amino acid sequence set forth in SEQ ID NO:25 or SEQ ID NO:25 with one substitution; and a VH CDR3 comprising or consisting of the amino acid sequence set forth in SEQ ID NO:5 or SEQ ID NO:5 with one substitution. In certain embodiments, the antibody or the antigen-binding fragment thereof comprises a VH CDR1 comprising or consisting of the amino acid sequence set forth in SEQ ID NO:23 or SEQ ID NO:23 with one substitution; a VH CDR2 comprising or consisting of the amino acid sequence set forth in SEQ ID NO:26 or SEQ ID NO:26 with one substitution; and a VH CDR3 comprising or consisting of the amino acid sequence set forth in SEQ ID NO:27 or SEQ ID NO:27 with one substitution. In certain embodiments, the antibody or the antigen-binding fragment thereof comprises a VH CDR1 comprising or consisting of the amino acid sequence set forth in SEQ ID NO:3; a VH CDR2 comprising or consisting of the amino acid sequence set forth in SEQ ID NO:4; and a VH CDR3 comprising or consisting of the amino acid sequence set forth in SEQ ID NO:5. In certain embodiments, the antibody or the antigen-binding fragment thereof comprises a VH CDR1 comprising or consisting of the amino acid sequence set forth in SEQ ID NO:21; a VH CDR2 comprising or consisting of the amino acid sequence set forth in SEQ ID NO:24; and a VH CDR3 comprising or consisting of the amino acid sequence set forth in SEQ ID NO:5. In certain embodiments, the antibody or the antigen-binding fragment thereof comprises a VH CDR1 comprising or consisting of the amino acid sequence set forth in SEQ ID NO:22; a VH CDR2 comprising or consisting of the amino acid sequence set forth in SEQ ID NO:25; and a VH CDR3 comprising or consisting of the amino acid sequence set forth in SEQ ID NO:5. In certain embodiments, the antibody or the antigen-binding fragment thereof comprises a VH CDR1 comprising or consisting of the amino acid sequence set forth in SEQ ID NO:23; a VH CDR2 comprising or consisting of the amino acid sequence set forth in SEQ ID NO:26; and a VH CDR3 comprising or consisting of the amino acid sequence set forth in SEQ ID NO:27.

In some embodiments of the above aspects, the antibody or the antigen-binding fragment thereof in addition to comprising the VH CDRs1, 2, and 3 described in the paragraph above, further comprises a VL CDR1 comprising or consisting of having the amino acid sequence set forth in SEQ ID NO:6 or SEQ ID NO:6 with two or fewer substitutions; a VL CDR2 comprising or consisting of having the amino acid sequence set forth in SEQ ID NO:7 or SEQ ID NO:7 with two or fewer substitutions; and a VL CDR3 comprising or consisting of having the amino acid sequence set forth in SEQ ID NO:8 or SEQ ID NO:8 with two or fewer substitutions. In certain other embodiments, the antibody or the antigen-binding fragment thereof in addition to comprising the VH CDRs1, 2, and 3 described in the paragraph above, further comprises a VL CDR1 comprising or consisting of having the amino acid sequence set forth in SEQ ID NO:28 or SEQ ID NO:28 with two or fewer substitutions; a VL CDR2 comprising or consisting of having the amino acid sequence set forth in SEQ ID NO:29 or SEQ ID NO:29 with two or fewer substitutions; and a VL CDR3 comprising or consisting of having the amino acid sequence set forth in SEQ ID NO:30 or SEQ ID NO:30 with two or fewer substitutions.

In some embodiments of the above aspects, the antibody or the antigen-binding fragment thereof is a humanized antibody comprising a VH CDR1 comprising or consisting of the amino acid sequence set forth in SEQ ID NO:3 or SEQ ID NO:3 with two or fewer substitutions; a VH CDR2 comprising or consisting of the amino acid sequence set forth in SEQ ID NO:4 or SEQ ID NO:4 with two or fewer substitutions; a VH CDR3 comprising or consisting of the amino acid sequence set forth in SEQ ID NO:5 or SEQ ID NO:5 with two or fewer substitutions; a VL CDR1 comprising or consisting of the amino acid sequence set forth in SEQ ID NO:6 or SEQ ID NO:6 with two or fewer substitutions; a VL CDR2 comprising or consisting of the amino acid sequence set forth in SEQ ID NO:7 or SEQ ID NO:7 with two or fewer substitutions; and a VL CDR3 comprising or consisting of the amino acid sequence set forth in SEQ ID NO:8 or SEQ ID NO:8 with two or fewer substitutions, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:21 or SEQ ID NO:21 with two or fewer substitutions; a VH CDR2 comprising or consisting of the amino acid sequence set forth in SEQ ID NO:24 or SEQ ID NO:24 with two or fewer substitutions; a VH CDR3 comprising or consisting of the amino acid sequence set forth in SEQ ID NO:5 or SEQ ID NO:5 with two or fewer substitutions; a VL CDR1 comprising or consisting of the amino acid sequence set forth in SEQ ID NO:6 or SEQ ID NO:6 with two or fewer substitutions; a VL CDR2 comprising or consisting of the amino acid sequence set forth in SEQ ID NO:7 or SEQ ID NO:7 with two or fewer substitutions; and a VL CDR3 comprising or consisting of the amino acid sequence set forth in SEQ ID NO:8 or SEQ ID NO:8 with two or fewer substitutions, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:22 or SEQ ID NO:22 with two or fewer substitutions; a VH CDR2 comprising or consisting of the amino acid sequence set forth in SEQ ID NO:25 or SEQ ID NO:25 with two or fewer substitutions; a VH CDR3 comprising or consisting of the amino acid sequence set forth in SEQ ID NO:5 or SEQ ID NO:5 with two or fewer substitutions; a VL CDR1 comprising or consisting of the amino acid sequence set forth in SEQ ID NO:6 or SEQ ID NO:6 with two or fewer substitutions; a VL CDR2 comprising or consisting of the amino acid sequence set forth in SEQ ID NO:7 or SEQ ID NO:7 with two or fewer substitutions; and a VL CDR3 comprising or consisting of the amino acid sequence set forth in SEQ ID NO:8 or SEQ ID NO:8 with two or fewer substitutions, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:23 or SEQ ID NO:23 with two or fewer substitutions; a VH CDR2 comprising or consisting of the amino acid sequence set forth in SEQ ID NO:26 or SEQ ID NO:26 with two or fewer substitutions; a VH CDR3 comprising or consisting of the amino acid sequence set forth in SEQ ID NO:27 or SEQ ID NO:27 with two or fewer substitutions; a VL CDR1 comprising or consisting of the amino acid sequence set forth in SEQ ID NO:28 or SEQ ID NO:28 with two or fewer substitutions; a VL CDR2 comprising or consisting of the amino acid sequence set forth in SEQ ID NO:29 or SEQ ID NO:29 with two or fewer substitutions; and a VL CDR3 comprising or consisting of the amino acid sequence set forth in SEQ ID NO:30 or SEQ ID NO:30 with two or fewer substitutions, wherein the effector function of the humanized antibody is reduced or eliminated.

In some embodiments of the above aspects, the antibody or the antigen-binding fragment thereof is a humanized antibody comprising a VH CDR1 comprising or consisting of the amino acid sequence set forth in SEQ ID NO:3 or SEQ ID NO:3 with one substitution; a VH CDR2 comprising or consisting of the amino acid sequence set forth in SEQ ID NO:4 or SEQ ID NO:4 with one substitution; a VH CDR3 comprising or consisting of the amino acid sequence set forth in SEQ ID NO:5 or SEQ ID NO:5 with one substitution; a VL CDR1 comprising or consisting of the amino acid sequence set forth in SEQ ID NO:6 or SEQ ID NO:6 with one substitution; a VL CDR2 comprising or consisting of the amino acid sequence set forth in SEQ ID NO:7 or SEQ ID NO:7 with one substitution; and a VL CDR3 comprising or consisting of the amino acid sequence set forth in SEQ ID NO:8 or SEQ ID NO:8 with one substitution, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:21 or SEQ ID NO:21 with one substitution; a VH CDR2 comprising or consisting of the amino acid sequence set forth in SEQ ID NO:24 or SEQ ID NO:24 with one substitution; a VH CDR3 comprising or consisting of the amino acid sequence set forth in SEQ ID NO:5 or SEQ ID NO:5 with one substitution; a VL CDR1 comprising or consisting of the amino acid sequence set forth in SEQ ID NO:6 or SEQ ID NO:6 with one substitution; a VL CDR2 comprising or consisting of the amino acid sequence set forth in SEQ ID NO:7 or SEQ ID NO:7 with one substitution; and a VL CDR3 comprising or consisting of the amino acid sequence set forth in SEQ ID NO:8 or SEQ ID NO:8 with one substitution, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:22 or SEQ ID NO:22 with one substitution; a VH CDR2 comprising or consisting of the amino acid sequence set forth in SEQ ID NO:25 or SEQ ID NO:25 with one substitution; a VH CDR3 comprising or consisting of the amino acid sequence set forth in SEQ ID NO:5 or SEQ ID NO:5 with one substitution; a VL CDR1 comprising or consisting of the amino acid sequence set forth in SEQ ID NO:6 or SEQ ID NO:6 with one substitution; a VL CDR2 comprising or consisting of the amino acid sequence set forth in SEQ ID NO:7 or SEQ ID NO:7 with one substitution; and a VL CDR3 comprising or consisting of the amino acid sequence set forth in SEQ ID NO:8 or SEQ ID NO:8 with one substitution, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:23 or SEQ ID NO:23 with one substitution; a VH CDR2 comprising or consisting of the amino acid sequence set forth in SEQ ID NO:26 or SEQ ID NO:26 with one substitution; a VH CDR3 comprising or consisting of the amino acid sequence set forth in SEQ ID NO:27 or SEQ ID NO:27 with one substitution; a VL CDR1 comprising or consisting of the amino acid sequence set forth in SEQ ID NO:28 or SEQ ID NO:28 with one substitution; a VL CDR2 comprising or consisting of the amino acid sequence set forth in SEQ ID NO:29 or SEQ ID NO:29 with one substitution; and a VL CDR3 comprising or consisting of the amino acid sequence set forth in SEQ ID NO:30 or SEQ ID NO:30 with one substitution, wherein the effector function of the humanized antibody is reduced or eliminated.

In some embodiments of the above aspects, the antibody or the antigen-binding fragment thereof is a humanized antibody comprising a VH CDR1 comprising or consisting of the amino acid sequence set forth in SEQ ID NO:3; a VH CDR2 comprising or consisting of the amino acid sequence set forth in SEQ ID NO:4; a VH CDR3 comprising or consisting of the amino acid sequence set forth in SEQ ID NO:5; a VL CDR1 comprising or consisting of the amino acid sequence set forth in SEQ ID NO:6; a VL CDR2 comprising or consisting of the amino acid sequence set forth in SEQ ID NO:7; and a VL CDR3 comprising or consisting of the amino acid sequence set forth in SEQ ID NO:8, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:21; a VH CDR2 comprising or consisting of the amino acid sequence set forth in SEQ ID NO:24; a VH CDR3 comprising or consisting of the amino acid sequence set forth in SEQ ID NO:5; a VL CDR1 comprising or consisting of the amino acid sequence set forth in SEQ ID NO:6; a VL CDR2 comprising or consisting of the amino acid sequence set forth in SEQ ID NO:7; and a VL CDR3 comprising or consisting of the amino acid sequence set forth in SEQ ID NO:8, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:22; a VH CDR2 comprising or consisting of the amino acid sequence set forth in SEQ ID NO:25; a VH CDR3 comprising or consisting of the amino acid sequence set forth in SEQ ID NO:5; a VL CDR1 comprising or consisting of the amino acid sequence set forth in SEQ ID NO:6; a VL CDR2 comprising or consisting of the amino acid sequence set forth in SEQ ID NO:7; and a VL CDR3 comprising or consisting of the amino acid sequence set forth in SEQ ID NO:8, wherein the effector function of the humanized antibody is reduced or eliminated. In certain embodiments, the antibody or the antigen-binding fragment thereof is a humanized antibody comprising a VH CDR1 comprising or consisting of the amino acid sequence set forth in SEQ ID NO:23; a VH CDR2 comprising or consisting of the amino acid sequence set forth in SEQ ID NO:26; a VH CDR3 comprising or consisting of the amino acid sequence set forth in SEQ ID NO:27; a VL CDR1 comprising or consisting of the amino acid sequence set forth in SEQ ID NO:28; a VL CDR2 comprising or consisting of the amino acid sequence set forth in SEQ ID NO:29; and a VL CDR3 comprising or consisting of the amino acid sequence set forth in SEQ ID NO:30, wherein the effector function of the humanized antibody is reduced or eliminated.

In some embodiments, the antibody or the antigen-binding fragment thereof is administered intravenously, subcutaneously, or intraarterially.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### DETAILED DESCRIPTION

This disclosure is based, at least in part, on the finding that an antibody that binds to the αvβ5 integrin is useful in treating acute kidney injury. In particular, in an animal model of acute kidney injury, an antibody that specifically binds to the αvβ5 integrin was found to reduce serum creatinine levels, an important indicator of renal health that is routinely used to determine if a subject has, or is at risk of developing, acute kidney injury. Accordingly, this disclosure features methods of treating or preventing acute kidney injury by administering an antibody or an antigen-binding fragment thereof that specifically binds the αvβ5 integrin or the β5 subunit of this integrin. In certain embodiments, the antibody or an antigen-binding fragment thereof is an αvβ5 antagonist (i.e., it competes with an αvβ5 ligand for available binding sites on the αvβ5 integrin).

Integrins are cell surface glycoprotein receptors that bind extracellular matrix proteins and mediate cell-cell and cell-extracellular matrix interactions, and cell-pathogen interactions. These receptors are composed of noncovalently associated alpha (α) and beta (β) chains that combine to give a variety of heterodimeric proteins with distinct cellular and adhesive specificities. These proteins can interact with cell surface ligands, transmembrane proteins, soluble proteases, pathogens, and growth factors. The importance of these receptors in biological processes is underscored by the pathological sequelae following integrin defects and from the often severe phenotypes of integrin subunit knockout animals.

The αvβ5 integrin is the only integrin that contains the β5 subunit. αvβ5 recognizes the RGD peptide sequence and binds vitronectin (Hynes, Cell, 69:11-25 (1992). αvβ5 can also bind fibronectin, osteopontin, tenascin c, and adenovirus penton c base. αvβ5 can activate TGF-β by a mechanism requiring an intact cytoskeleton and cell contraction. αv and β5 have both been sequenced and characterized (Hynes, 1992 supra and U.S. Pat. No. 5,527,679, respectively).

β5

The amino acid sequence of the human β5 protein (Genbank® Accession No. NP_{—}002204.2) is shown below:

(SEQ ID NO: 1)
MPRAPAPLYA CLLGLCALLP RLAGLNICTS GSATSCEECL
LIHPKCAWCS KEDFGSPRSI TSRCDLRANL VKNGCGGEIE
SPASSFHVLR SLPLSSKGSG SAGWDVIQMT PQEIAVNLRP
GDKTTFQLQV RQVEDYPVDL YYLMDLSLSM KDDLDNIRSL
GTKLAEEMRK LTSNFRLGFG SFVDKDISPF SYTAPRYQTN
PCIGYKLFPN CVPSFGFRHL LPLTDRVDSF NEEVRKQRVS
RNRDAPEGGF DAVLQAAVCK EKIGWRKDAL HLLVFTTDDV
PHIALDGKLG GLVQPHDGQC HLNEANEYTA SNQMDYPSLA
LLGEKLAENN INLIFAVTKN HYMLYKNFTA LIPGTTVEIL
DGDSKNIIQL IINAYNSIRS KVELSVWDQP EDLNLFFTAT
CQDGVSYPGQ RKCEGLKIGD TASFEVSLEA RSCPSRHTEH
VFALRPVGFR DSLEVGVTYN CTCGCSVGLE PNSARCNGSG
TYVCGLCECS PGYLGTRCEC QDGENQSVYQ NLCREAEGKP
LCSGRGDCSC NQCSCFESEF GKIYGPFCEC DNFSCARNKG
VLCSGHGECH CGECKCHAGY IGDNCNCSTD ISTCRGRDGQ
ICSERGHCLC GQCQCTEPGA FGEMCEKCPT CPDACSTKRD
CVECLLLHSG KPDNQTCHSL CRDEVITWVD TIVKDDQEAV
LCFYKTAKDC VMMFTYVELP SGKSNLTVLR EPECGNTPNA
MTILLAVVGS ILLVGLALLA IWKLLVTIHD RREFAKFQSE
RSRARYEMAS NPLYRKPIST HTVDFTFNKF NKSYNGTVD

The amino acid sequence of the murine β5 protein (Genbank® Accession No. NP_{—}001139356.1) is shown below:

(SEQ ID NO: 2)
MPRVPATLYACLLGLCALVP RLAGLNICTSGSATSCEECL
LIHPKCAWCS KEYFGNPRSI TSRCDLKANLIRNGCEGEIE
SPASSTHVLR NLPLSSKGSSATGSDVIQMT PQEIAVSLRP
GEQTTFQLQVRQVEDYPVDLYYLMDLSLSM KDDLENIRSLGTKLAEEM
RK LTSNFRLGFG SFVDKDISPFSYTAPRYQTNPCIGYKLFPN
CVPSFGFRHLLPLTDRVDSFNEEVRKQRVS RNRDAPEGGF
DAVLQAAVCKEKIGWRKDALHLLVFTTDDVPHIALDGKLGGLVQPHDGQC
HLNEANEYTASNQMDYPSLALLGEKLAENNINLIFAVTKN HYMLYKNFT
ALIPGTTVEIL HGDSKNIIQLIINAYSSIRA KVELSVWDQP EDLNLF
FTATCQDGISYPGQRKCEGLKIGD TASFEVSVEARSCPGRQAAQ
SFTLRPVGFR DSLQVEVAYN CTCGCSTGLE PNSARCSGNG
TYTCGLCECD PGYLGTRCEC QEGENQSGYQ NLCREAEGKP
LCSGRGECSC NQCSCFESEF GRIYGPFCEC DSFSCARNKG
VLCSGHGECH CGECKCHAGY IGDNCNCSTD VSTCRAKDGQ
ICSDRGRCVC GQCQCTEPGA FGETCEKCPT CPDACSSKRD
CVECLLLHQG KPDNQTCHHQ CKDEVITWVD TIVKDDQEAV
LCFYKTAKDC VMMFSYTELP NGRSNLTVLR EPECGSAPNA
MTILLAVVGS ILLIGMALLA IWKLLVTIHD RREFAKFQSE
RSRARYEMAS NPLYRKPIST HTVDFAFNKF NKSYNGSVD

Anti-αvβ5 Antibodies

This disclosure encompasses the use of an antibody or antigen-binding fragment thereof that specifically binds the αvβ5 integrin and/or the β5 subunit of the integrin for the treatment or prevention of acute kidney injury. In certain embodiments, the antibody or antigen-binding fragment competes with an αvβ5 ligand for available ligand binding sites on the αvβ5 integrin. In some embodiments, the antibody is a humanized form of the murine ALULA antibody produced by the hybridoma deposited at the ATCC on Feb. 13, 2004, with the accession number PTA-5817.

The amino acid sequence of the murine ALULA antibody's heavy chain variable region (VH) amino acid sequence is provided below:

(SEQ ID NO: 9)
EVQVQQSGTVLARPGASVKMSCKASGYTFTSYWMHWVKQRPGQGLEWIGA
IYPGNSDTSYNQKFKGKAKLTAVTSPNTAYMELSSLTNEDSAVYYCTTTT
YGYDWFAYWGQGTLVTVSA

The nucleic acid sequence encoding the murine ALULA antibody's VH nucleic acid is provided below:

(SEQ ID NO: 10)
GAGGTTCAGGTCCAGCAGTCTGGGACTGTGCTGGCAAGGCCTGGGGCTTC
AGTGAAGATGTCCTGCAAGGCTTCTGGCTACACCTTTACCAGCTACTGGA
TGCACTGGGTAAAACAGAGGCCTGGACAGGGTCTGGAATGGATTGGCGCT
ATTTATCCTGGAAATAGTGATACTAGCTACAACCAGAAGTTCAAGGGCAA
GGCCAAACTGACTGCAGTCACATCCCCCAACACTGCCTACATGGAGCTCA
GCAGCCTGACAAATGAGGACTCTGCGGTCTATTACTGTACAACCACTACA
TATGGTTACGACTGGTTTGCTTACTGGGGCCAAGGGACTCTGGTCACTGT
CTCTGCA

The amino acid sequence of the murine ALULA antibody's light chain variable region (VL) amino acid sequence is provided below:

(SEQ ID NO: 11)
NIMMTQSPSSLTVSAGEKVTMSCKSSQSVLYSSNQKNYLAWYQQKPGQSP
KLLIYWASTRESGVPDRFTGSGSGTDFTLTISSVQAEDLAVYYCHQYLSS
LTFGAGTKLELK

The nucleic acid sequence encoding the murine ALULA VL nucleic acid sequence is provided below:

(SEQ ID NO: 12)
AACATTATGATGACACAGTCGCCATCATCTCTGACTGTGTCTGCAGGAGA
AAAGGTCACTATGAGCTGTAAGTCCAGTCAAAGTGTTTTATACAGTTCAA
ATCAGAAGAACTACTTGGCCTGGTACCAGCAGAAACCAGGGCAGTCTCCT
AAACTGCTGATCTACTGGGCATCCACTAGGGAATCTGGTGTCCCTGATCG
CTTCACAGGCAGTGGATCTGGGACAGATTTTACTCTTACCATCAGCAGTG
TACAAGCTGAAGACCTGGCAGTTTATTACTGTCATCAATACCTCTCCTCG
CTCACGTTCGGTGCTGGGACCAAGCTGGAGCTGAAA

The amino acid sequences of the complementarity determining regions (CDRs) 1, 2, and 3 of the heavy chain variable region and the light chain variable region of ALULA are provided below. The CDRs are based upon the Kabat numbering system.

| Domain | SEQ ID NO | Sequence |
|---|---|---|
| VH CDR1 | 3 | SYWMH |
| VH CDR2 | 4 | AIYPGNSDTSYNQKFKG |
| VH CDR3 | 5 | TTYGYDWFAY |
| VL CDR1 | 6 | KSSQSVLYSSNQKNYLA |
| VL CDR2 | 7 | WASTRES |
| VL CDR3 | 8 | HQYLSSLT |

This application also discloses “alternate CDRs” of ALULA that can be used in the antibodies of this invention. By “alternate” CDRs are meant CDRs (CDR1, CDR2, and CDR3) defined according to any one of the Chothia from AbYsis, enhanced Chothia/AbM CDR, or the contact definitions. These alternate CDRs can be obtained, e.g., by using the AbYsis database (www.bioinf.org.uk/abysis/sequence_input/key_annotation/key_annotation.cgi). The amino acid sequences of “alternate” CDRs 1, 2, and 3 of the heavy chain variable region and the light chain variable region of ALULA are compared with the CDRs defined according to Kabat in the Table below.

| | | | Enhanced Chothia/ | |
|---|---|---|---|---|
| | | | AbM CDR | |
| Domain | Kabat Defintion | Chothia from AbYsis | Definition | Contact Definition |
| VH CDR1 | SYWMH | GYTFTSY | GYTFTSYWMH | TSYWMH |
| | (SEQ ID NO: 3) | (SEQ ID NO: 21) | (SEQ ID NO: 22) | (SEQ ID NO: 23) |
| VH CDR2 | AIYPGNSDTSYNQKFKG | YPGNSD | AIYPGNSDTS | WIGAIYPGNSDTS |
| | (SEQ ID NO: 4) | (SEQ ID NO: 24) | (SEQ ID NO: 25) | (SEQ ID NO: 26) |
| VH CDR3 | TTYGYDWFAY | TTYGYDWFAY | TTYGYDWFAY | TTTTYGYDWFA |
| | (SEQ ID NO: 5) | (SEQ ID NO: 5) | (SEQ ID NO: 5) | (SEQ ID NO: 27) |
| VL CDR1 | KSSQSVLYSSNQKNYLA | KSSQSVLYSSNQKNYLA | KSSQSVLYSSNQKNYLA | LYSSNQKNYLAWY |
| | (SEQ ID NO: 6) | (SEQ ID NO: 6) | (SEQ ID NO: 6) | (SEQ ID NO: 28) |
| VL CDR2 | WASTRES | WASTRES | WASTRES | LLIYWASTRE |
| | (SEQ ID NO: 7) | (SEQ ID NO: 7) | (SEQ ID NO: 7) | (SEQ ID NO: 29) |
| VL CDR3 | HQYLSSLT | HQYLSSLT | HQYLSSLT | HQYLSSL |
| | (SEQ ID NO: 8) | (SEQ ID NO: 8) | (SEQ ID NO: 8) | (SEQ ID NO: 30) |

In certain embodiments, the anti-αvβ5 (or anti-β5) antibody or antigen-binding fragment thereof is humanized. In one embodiment, the antibody or antigen-binding fragment thereof is humanized ALULA. In certain embodiments, the antibody or antigen-binding fragment thereof comprises an amino acid sequence that is at least 85%, 86%, 87%, 88%, 89%, 90%, 91%, 92%, 93%, 94%, 95%, 96%, 97%, 98%, or 99% identical to the amino acid sequence set forth in SEQ ID NO:9. In some embodiments, such antibodies or antigen-binding fragments that specifically bind αvβ5 (or the β5 subunit), inhibit the interaction between αvβ5 and vitronectin; inhibit the interaction between αvβ5 and LAP of TGF-β; and/or inhibit the activation of TGF-β. In certain embodiments, the anti-αvβ5 (or anti-β5) antibody or antigen-binding fragment thereof further comprises an amino acid sequence that is at least 85%, 86%, 87%, 88%, 89%, 90%, 91%, 92%, 93%, 94%, 95%, 96%, 97%, 98%, or 99% identical to the amino acid sequence set forth in SEQ ID NO:11. In some embodiments, where the antibody or antigen-binding fragment thereof comprises both a heavy chain variable region and a light chain variable region, the antibody or antigen-binding fragment specifically binds αvβ5 (or the β5 subunit), inhibits the interaction between αvβ5 and vitronectin; inhibits the interaction between αvβ5 and LAP of TGF-β; and/or inhibits the activation of TGF-β.

In some embodiments, the anti-αvβ5 (or anti-β5) antibody or antigen-binding fragment thereof comprises one or more, two, or all three of CDR1 (SEQ ID NO:3), CDR2 (SEQ ID NO:4), and CDR3 (SEQ ID NO:5) of the VH of ALULA. In some embodiments, such antibodies or antigen-binding fragments that specifically bind αvβ5 (or the β5 subunit), inhibit the interaction between αvβ5 and vitronectin; inhibit the interaction between αvβ5 and LAP of TGF-β; and/or inhibit the activation of TGF-β. In certain embodiments, the anti-αvβ5 (or anti-β5) antibody or antigen-binding fragment thereof further comprises one or more, two, or all three of CDR1 (SEQ ID NO:6), CDR2 (SEQ ID NO:7), and CDR3 (SEQ ID NO:8) of the VL of ALULA. In some embodiments, such antibodies or antigen-binding fragments that specifically bind αvβ5 (or the β5 subunit), inhibit the interaction between αvβ5 and vitronectin; inhibit the interaction between αvβ5 and LAP of TGF-β; and/or inhibit the activation of TGF-β. In a specific embodiment, the anti-αvβ5 (or anti-β5) antibody or antigen-binding fragment thereof comprises CDR1 (SEQ ID NO:3), CDR2 (SEQ ID NO:4), and CDR3 (SEQ ID NO:5) of the VH of ALULA and CDR1 (SEQ ID NO:6), CDR2 (SEQ ID NO:7), and CDR3 (SEQ ID NO:8) of the VL of ALULA. In another embodiment, the anti-αvβ5 (or anti-β5) antibody or antigen-binding fragment thereof comprises ALULA's VH CDR1 (SEQ ID NO:3) with two or fewer substitutions, VH CDR2 (SEQ ID NO:4) with two or fewer substitutions, and VH CDR3 (SEQ ID NO:5) with two or fewer substitutions. In a further embodiment, the anti-αvβ5 (or anti-β5) antibody or antigen-binding fragment thereof comprises ALULA's VL CDR1 (SEQ ID NO:6) with two or fewer substitutions, VL CDR2 (SEQ ID NO:7) with two or fewer substitutions, and VL CDR3 (SEQ ID NO:8) with two or fewer substitutions. In some embodiments, such antibodies or antigen-binding fragments that specifically bind αvβ5 (or the β5 subunit), inhibit the interaction between αvβ5 and vitronectin; inhibit the interaction between αvβ5 and LAP of TGF-β; and/or inhibit the activation of TGF-β.

In some embodiments, the anti-αvβ5 (or anti-β5) antibody or antigen-binding fragment thereof comprises one or more, two, or all three of an alternate CDR1 (SEQ ID NO:21, 22, or 23), an alternate CDR2 (SEQ ID NO:24, 25, or 26), and alternate CDR3 (SEQ ID NO:5 or 27) of the VH of ALULA. In some embodiments, such antibodies or antigen-binding fragments that specifically bind αvβ5 (or the β5 subunit), inhibit the interaction between αvβ5 and vitronectin; inhibit the interaction between αvβ5 and LAP of TGF-β; and/or inhibit the activation of TGF-β. In certain embodiments, the anti-αvβ5 (or anti-β5) antibody or antigen-binding fragment thereof further comprises one or more, two, or all three of an alternate CDR1 (SEQ ID NO:6 or 28), an alternate CDR2 (SEQ ID NO:7 or 29), and an alternate CDR3 (SEQ ID NO:8 or 30) of the VL of ALULA. In some embodiments, such antibodies or antigen-binding fragments that specifically bind αvβ5 (or the β5 subunit), inhibit the interaction between αvβ5 and vitronectin; inhibit the interaction between αvβ5 and LAP of TGF-β; and/or inhibit the activation of TGF-β. In one embodiment, the anti-αvβ5 (or anti-β5) antibody or antigen-binding fragment thereof comprises alternate CDR1 (SEQ ID NO:21), alternate CDR2 (SEQ ID NO:24), and alternate CDR3 (SEQ ID NO:5) of the VH of ALULA and/or alternate CDR1 (SEQ ID NO:6), alternate CDR2 (SEQ ID NO:7), and alternate CDR3 (SEQ ID NO:8) of the VL of ALULA. In another embodiment, the anti-αvβ5 (or anti-β5) antibody or antigen-binding fragment thereof comprises alternate CDR1 (SEQ ID NO:22), alternate CDR2 (SEQ ID NO:25), and alternate CDR3 (SEQ ID NO:5) of the VH of ALULA and/or alternate CDR1 (SEQ ID NO:6), alternate CDR2 (SEQ ID NO:7), and alternate CDR3 (SEQ ID NO:8) of the VL of ALULA. In a further embodiment, the anti-αvβ5 (or anti-β5) antibody or antigen-binding fragment thereof comprises alternate CDR1 (SEQ ID NO:23), alternate CDR2 (SEQ ID NO:26), and alternate CDR3 (SEQ ID NO:27) of the VH of ALULA and/or alternate CDR1 (SEQ ID NO:28), alternate CDR2 (SEQ ID NO:29), and alternate CDR3 (SEQ ID NO:30) of the VL of ALULA. In another embodiment, the anti-αvβ5 (or anti-β5) antibody or antigen-binding fragment thereof comprises ALULA's VH alternate CDR1 (SEQ ID NO:21, 22, or 23) with two or fewer substitutions (e.g., 2, 1, or none), VH alternate CDR2 (SEQ ID NO:24, 25, or 26), with two or fewer substitutions, and VH alternate CDR3 (SEQ ID NO:5 or 27) with two or fewer substitutions. In a further embodiment, the anti-αvβ5 (or anti-β5) antibody or antigen-binding fragment thereof comprises ALULA's VL alternate CDR1 (SEQ ID NO:6 or 28) with two or fewer substitutions, VL alternate CDR2 (SEQ ID NO:7 or 29) with two or fewer substitutions, and VL alternate CDR3 (SEQ ID NO:8 or 30) with two or fewer substitutions. In some embodiments, such antibodies or antigen-binding fragments that specifically bind αvβ5 (or the β5 subunit), inhibit the interaction between αvβ5 and vitronectin; inhibit the interaction between αvβ5 and LAP of TGF-β; and/or inhibit the activation of TGF-β.

This disclosure also includes antibodies that specifically bind αvβ5 and/or β5 that have six or fewer (e.g., six, five, four, three or fewer, three, two or fewer, two, or one) amino acid substitutions in one, two, three, or all four of the framework regions, and/or four or fewer (e.g., four, three or fewer, two or fewer, or one) amino acid substitutions in one, two, or all three CDRs (or alternate CDRs), of the heavy chain variable region of ALULA. The application also includes antibodies that have six or fewer (e.g., six, five, four, three or fewer, three, two or fewer, two, or one) amino acid substitutions in one, two, three, or all four of the framework regions, and/or four or fewer (e.g., four, three or fewer, three, two or fewer, two, or one) amino acid substitutions in one, two, or all three CDRs (or alternate CDRs), of the light chain variable region of ALULA. In some embodiments, the amino acid substitutions are conservative amino acid substitutions. In some embodiments, such antibodies or antigen-binding fragments that specifically bind αvβ5 (or the β5 subunit), inhibit the interaction between αvβ5 and vitronectin; inhibit the interaction between αvβ5 and LAP of TGF-β; and/or inhibit the activation of TGF-β.

This disclosure also encompasses the use of any antibody or antigen-binding fragment thereof that competes with an antibody that specifically binds the αvβ5 integrin and/or the β5 subunit of the integrin (e.g., ALULA or humanized forms of ALULA) for the treatment or prevention of acute kidney injury or any other indication described herein.

In certain embodiments the VH and or VL region can be linked to a constant region (e.g., a wild-type human Fc region or an Fc region that includes one or more alterations). In certain embodiments, the constant region comprises a CH1 domain and a hinge region. In some embodiments, the constant region comprises a CH3 domain. In some embodiments, the antibody has a constant region derived from a human kappa or lambda sequence. In a specific embodiment, the constant region comprises a human subgroup kappa 2 sequence. The constant region can be a human Fc region, e.g., a wild-type Fc region, or an Fc region that includes one or more amino acid substitutions. The constant region can have substitutions that modify the properties of the antibody (e.g., increase or decrease one or more of: Fc receptor binding, antibody glycosylation, the number of cysteine residues, effector cell function, or complement function). For example, the human IgG1 constant region can be mutated at one or more residues, e.g., one or more of residues 234 and 237 (based on Kabat numbering). Antibodies may have mutations in the CH2 region of the heavy chain that reduce or alter effector function, e.g., Fc receptor binding and complement activation. For example, antibodies may have mutations such as those described in U.S. Pat. Nos. 5,624,821 and 5,648,260. Antibodies may also have mutations that stabilize the disulfide bond between the two heavy chains of an immunoglobulin, such as mutations in the hinge region of IgG4, as disclosed in the art (e.g., Angal et al., Mol. Immunol., 30:105-08 (1993)). See also, e.g., U.S. 2005-0037000. In certain embodiments, the antibody has an isotype selected from the group consisting of IgG1, IgG2, IgG3, and IgG4.

In certain embodiments the antibodies or antigen-binding fragments thereof can be conjugated to one or more agents that are useful for treating or preventing acute kidney injury or any other indication described herein. These agents can be, e.g., micro RNAs, micro RNA mimics, small interfering RNAs, antagomirs, antisense nucleic acids, ribozymes, small molecule compounds, and other chemical moieities. Such antibodies or antigen-binding fragments can be used e.g., to deliver the bound agent(s) to a cell or tissue of interest that expresses αvβ5. In certain embodiments, the antibodies or antigen-binding fragments thereof can be conjugated to a drug that needs to be selectively delivered to αvβ5 expressing cells (e.g., to decrease or prevent the toxicity of the conjugated drug; to decrease side effects).

Antibodies can be selected for use based on improved potency, higher affinity or avidity for αvβ5, and/or reduced immunogenicity than previously known αvβ5 antibodies. Methods of determining potency, affinity or avidity, and immunogenicity of antibodies are within the skill of the ordinary artisan.

Methods of Obtaining Anti-αvβ5 Antibodies

Numerous methods are available for obtaining antibodies, particularly human antibodies. One exemplary method includes screening protein expression libraries, e.g., phage or ribosome display libraries. Phage display is described, for example, in U.S. Pat. No. 5,223,409; Smith, Science 228:1315-1317 (1985); WO 92/18619; WO 91/17271; WO 92/20791; WO 92/15679; WO 93/01288; WO 92/01047; WO 92/09690; and WO 90/02809. The display of Fab's on phage is described, e.g., in U.S. Pat. Nos. 5,658,727; 5,667,988; and 5,885,793.

In addition to the use of display libraries, other methods can be used to obtain a αvβ5-binding antibody. For example, the β5 protein or a peptide thereof can be used as an antigen in a non-human animal, e.g., a rodent, e.g., a mouse, hamster, or rat. In addition, cells transfected with a cDNA encoding β5 can be injected into a non-human animal as a means of producing antibodies that effectively bind the cell surface αvβ5 protein.

In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al., Nature Genetics 7:13-21 (1994), U.S. 2003-0070185, WO 96/34096, and WO 96/33735.

In another embodiment, a monoclonal antibody is obtained from the non-human animal, and then modified, e.g., humanized or deimmunized. Winter describes an exemplary CDR-grafting method that may be used to prepare humanized antibodies described herein (U.S. Pat. No. 5,225,539). All or some of the CDRs of a particular human antibody may be replaced with at least a portion of a non-human antibody. It may only be necessary to replace the CDRs required for binding or binding determinants of such CDRs to arrive at a useful humanized antibody that binds to αvβ5.

Humanized antibodies can be generated by replacing sequences of the Fv variable region that are not directly involved in antigen-binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16: 237-242 (1995); Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

A non-human αvβ5-binding antibody may also be modified by specific deletion of human T cell epitopes or “deimmunization” by the methods disclosed in WO 98/52976 and WO 00/34317. Briefly, the heavy and light chain variable regions of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the V_{H} and V_{L} sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable regions, or preferably, by single amino acid substitutions. As far as possible, conservative substitutions are made. Often, but not exclusively, an amino acid common to a position in human germline antibody sequences may be used. After the deimmunizing changes are identified, nucleic acids encoding V_{H} and V_{L} can be constructed by mutagenesis or other synthetic methods (e.g., de novo synthesis, cassette replacement, and so forth). A mutagenized variable sequence can, optionally, be fused to a human constant region, e.g., human IgG1 or kappa constant regions.

In some cases, a potential T cell epitope will include residues known or predicted to be important for antibody function. For example, potential T cell epitopes are usually biased towards the CDRs. In addition, potential T cell epitopes can occur in framework residues important for antibody structure and binding. Changes to eliminate these potential epitopes will in some cases require more scrutiny, e.g., by making and testing chains with and without the change. Where possible, potential T cell epitopes that overlap the CDRs can be eliminated by substitutions outside the CDRs. In some cases, an alteration within a CDR is the only option, and thus variants with and without this substitution can be tested. In other cases, the substitution required to remove a potential T cell epitope is at a residue position within the framework that might be critical for antibody binding. In these cases, variants with and without this substitution are tested. Thus, in some cases several variant deimmunized heavy and light chain variable regions are designed and various heavy/light chain combinations are tested to identify the optimal deimmunized antibody. The choice of the final deimmunized antibody can then be made by considering the binding affinity of the different variants in conjunction with the extent of deimmunization, particularly, the number of potential T cell epitopes remaining in the variable region. Deimmunization can be used to modify any antibody, e.g., an antibody that includes a non-human sequence, e.g., a synthetic antibody, a murine antibody other non-human monoclonal antibody, or an antibody isolated from a display library.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and 6,407,213; Tempest et al. (1991) Biotechnology 9:266-271. Still another method is termed “humaneering” and is described, for example, in U.S. 2005-008625.

Antibodies, such as those described above, can be made, for example, by preparing and expressing synthetic genes that encode the recited amino acid sequences. Methods of generating variants (e.g., comprising amino acid substitutions) of any of the anti-αvβ5 antibodies are well known in the art. These methods include, but are not limited to, preparation by site-directed (or oligonucleotide-mediated) mutagenesis, PCR mutagenesis, and cassette mutagenesis of a prepared DNA molecule encoding the antibody or any portion thereof (e.g., a framework region, a CDR, a constant region). Site-directed mutagenesis is well known in the art (see, e.g., Carter et al., Nucl. Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nucl. Acids Res. 17:723-733 (1989). Another method for preparing sequence variants, cassette mutagenesis, is based on the technique described by Wells et al., Gene, 34:315-323 (1985).

### Affinity Maturation

In one embodiment, an anti-αvβ5 antibody or antigen-binding fragment thereof described herein is modified, e.g., by mutagenesis, to provide a pool of modified antibodies. The modified antibodies are then evaluated to identify one or more antibodies having altered functional properties (e.g., improved binding, improved stability, reduced antigenicity, or increased stability in vivo). In one implementation, display library technology is used to select or screen the pool of modified antibodies. Higher affinity antibodies are then identified from the second library, e.g., by using higher stringency or more competitive binding and washing conditions. Other screening techniques can also be used.

In some implementations, the mutagenesis is targeted to regions known or likely to be at the binding interface. If, for example, the identified binding proteins are antibodies, then mutagenesis can be directed to the CDR regions (or alternate CDR regions) of the heavy or light chains as described herein. Further, mutagenesis can be directed to framework regions near or adjacent to the CDRs, e.g., framework regions, particularly within 10, 5, or 3 amino acids of a CDR (or alternate CDR) junction. In the case of antibodies, mutagenesis can also be limited to one or a few of the CDRs (or alternate CDRs), e.g., to make step-wise improvements.

In one embodiment, mutagenesis is used to make an antibody more similar to one or more germline sequences. One exemplary germlining method can include: identifying one or more germline sequences that are similar (e.g., most similar in a particular database) to the sequence of the isolated antibody. Then mutations (at the amino acid level) can be made in the isolated antibody, either incrementally, in combination, or both. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a CDR (or alternate CDR) region. For example, the germline CDR (or alternate CDR) residue can be from a germline sequence that is similar (e.g., most similar) to the variable region being modified. After mutagenesis, activity (e.g., binding or other functional activity) of the antibody can be evaluated to determine if the germline residue or residues are tolerated. Similar mutagenesis can be performed in the framework regions.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity, relative to the donor non-human antibody. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may include using two germline sequences that separately contribute to the amino-terminal portion and the carboxy-terminal portion. In other implementations, more than one or two germline sequences are used, e.g., to form a consensus sequence.

Calculations of “sequence identity” between two sequences are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In other embodiments, the antibody may be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies may be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Crit. Rev. Biochem., 22:259-306. Removal of any carbohydrate moieties present on the antibodies may be accomplished chemically or enzymatically as described in the art (Hakimuddin et al. (1987) Arch. Biochem. Biophys., 259:52; Edge et al. (1981) Anal. Biochem., 118:131; and Thotakura et al. (1987) Meth. Enzymol., 138:350). See, e.g., U.S. Pat. No. 5,869,046 for a modification that increases in vivo half life by providing a salvage receptor binding epitope.

In one embodiment, an antibody has CDR sequences (e.g., a Chothia or Kabat CDR) that differ from those of SEQ ID NOs: 3, 4, 5, 6, 7, and 8. CDR sequences that differ from those of the humanized ALULA antibodies described herein include amino acid changes, such as substitutions of 1, 2, 3, or 4 amino acids if a CDR is 5-7 amino acids in length, or substitutions of 1, 2, 3, 4, 5, 6, or 7 of amino acids in the sequence of a CDR if a CDR is 10 amino acids or greater in length. The amino acid that is substituted can have similar charge, hydrophobicity, or stereochemical characteristics. In some embodiments, the amino acid substitution(s) is a conservative substitution. In other embodiments, the amino acid substitution(s) is a non-conservative substitution. Such substitutions are within the ordinary skill of an artisan. The antibody or antibody fragments thereof that contain the substituted CDRs can be screened to identify antibodies having one or more of the features described herein (e.g., specifically binding to β5, inhibiting the binding of αvβ5 to vitronectin).

Unlike in CDRs, more substantial changes in structure framework regions (FRs) can be made without adversely affecting the binding properties of an antibody. Changes to FRs include, but are not limited to, humanizing a nonhuman-derived framework or engineering certain framework residues that are important for antigen contact or for stabilizing the binding site, e.g., changing the class or subclass of the constant region, changing specific amino acid residues which might alter an effector function such as Fc receptor binding (Lund et al., J. Immun., 147:2657-62 (1991); Morgan et al., Immunology, 86:319-24 (1995)), or changing the species from which the constant region is derived.

The anti-αvβ5 antibodies can be in the form of full length antibodies, or in the form of low molecular weight forms (e.g., biologically active antibody fragments or minibodies) of the anti-αvβ5 antibodies, e.g., Fab, Fab′, F(ab′)₂, Fv, Fd, dAb, scFv, and sc(Fv)2. Other anti-αvβ5 antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Microbiol. Biotechnol., 77(1):13-22 (2007); U.S. 2005/0079574 and Davies et al. (1996) Protein Eng., 9(6):531-7. Like a whole antibody, a sdAb is able to bind selectively to a specific antigen. With a molecular weight of only 12-15 kDa, sdAbs are much smaller than common antibodies and even smaller than Fab fragments and single-chain variable fragments.

Provided herein are compositions comprising a mixture of an anti-αvβ5 antibody or antigen-binding fragment thereof and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising an anti-αvβ5 antibody or antigen-binding fragment thereof comprising at least one deamidation site, wherein the pH of the composition is from about 5.0 to about 6.5, such that, e.g., at least about 90% of the anti-αvβ5 antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g. as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e. the neutral amide side chain has been converted to a residue with an overall acidic character.

The term “mixture” as used herein in reference to a composition comprising an anti-αvβ5 antibody or antigen-binding fragment thereof, means the presence of both the desired anti-αvβ5 antibody or antigen-binding fragment thereof and one or more acidic variants thereof. The acidic variants may comprise predominantly deamidated anti-αvβ5 antibody, with minor amounts of other acidic variant(s).

In certain embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

In certain embodiments, an anti-αvβ5 antibody or antigen-binding fragment thereof or low molecular weight antibodies thereof specifically binds to β5, inhibits the binding of αvβ5 to vitronectin, inhibits the binding of αvβ5 to LAP of TGF-β, inhibits the activation of TGF-β, inhibits TGF-β signaling, and/or reduces the severity of symptoms when administered to human patients having, or animal models of acute kidney injury (e.g., Heyman et al., Contrin. Nephrol., 169:286-296 (2011); Heyman et al., Exp. Opin. Drug Disc., 4(6): 629-641 (2009); Morishita et al., Ren. Fail., 33(10):1013-1018 (2011); Wei Q et al., Am. J. Physiol. Renal Physiol., 303(11):F1487-94 (2012)). In one embodiment, the anti-αvβ5 antibody or antigen-binding fragment thereof or low molecular weight antibodies thereof inhibit disease development in a rat unilateral renal ischemic clamp model. These features of an anti-αvβ5 antibody or antigen-binding fragment thereof or low molecular weight antibodies thereof can be measured according to methods known in the art.

### Antibody Fragments

Antibody fragments (e.g., Fab, Fab′, F(ab′)2, Facb, and Fv) of αvβ5-binding antibodies may be prepared by proteolytic digestion of intact αvβ5 antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Pluckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)2 fragments (Carter et al., Bio/Technology, 10:163-167 (1992)). According to another approach, F(ab′)2 fragments can be isolated directly from recombinant host cell culture. Fab and F(ab′) 2 fragment with increased in vivo half-life comprising a salvage receptor binding epitope residues are described in U.S. Pat. No. 5,869,046.

### Minibodies

Minibodies of anti-αvβ5 antibodies include diabodies, single chain (scFv), and single-chain (Fv)2 (sc(Fv)2).

A “diabody” is a bivalent minibody constructed by gene fusion (see, e.g., Holliger, P. et al., Proc. Natl. Acad. Sci. U.S.A, 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see e.g., Huston et al., Proc. Natl. Acad. Sci. U.S.A, 85:5879-5883 (1988); and Pluckthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The H chain V region and L chain V region in an scFv may be derived from any anti-αvβ5 antibody or antigen-binding fragment thereof described herein.

An sc(Fv)2 is a minibody in which two VHs and two VLs are linked by a linker to form a single chain (Hudson, et al., J. Immunol. Methods, (1999) 231: 177-189 (1999)). An sc(Fv)2 can be prepared, for example, by connecting scFvs with a linker. The sc(Fv)2 of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however the order of the two VHs and two VLs is not limited to the above arrangement, and they may be arranged in any order. Examples of arrangements are listed below:

[VL] linker [VH] linker [VH] linker [VL]

[VH] linker [VL] linker [VL] linker [VH]

[VH] linker [VH] linker [VL] linker [VL]

[VL] linker [VL] linker [VH] linker [VH]

[VL] linker [VH] linker [VL] linker [VH]

Normally, three linkers are required when four antibody variable regions are linked; the linkers used may be identical or different. There is no particular limitation on the linkers that link the VH and VL regions of the minibodies. In some embodiments, the linker is a peptide linker. Any arbitrary single-chain peptide comprising about three to 25 residues (e.g., 5, 6, 7, 8, 9, 10, 11, 12, 13, 14, 15, 16, 17, 18) can be used as a linker. Examples of such peptide linkers include: Ser; Gly Ser; Gly Gly Ser; Ser Gly Gly; Gly Gly Gly Ser (SEQ ID NO:13); Ser Gly Gly Gly (SEQ ID NO:14); Gly Gly Gly Gly Ser (SEQ ID NO:15); Ser Gly Gly Gly Gly (SEQ ID NO:16); Gly Gly Gly Gly Gly Ser (SEQ ID NO:17); Ser Gly Gly Gly Gly Gly (SEQ ID NO:18); Gly Gly Gly Gly Gly Gly Ser (SEQ ID NO:19); Ser Gly Gly Gly Gly Gly Gly (SEQ ID NO:20); (Gly Gly Gly Gly Ser (SEQ ID NO:15)ₙ, wherein n is an integer of one or more; and (Ser Gly Gly Gly Gly (SEQ ID NO:16)ₙ, wherein n is an integer of one or more.

In certain embodiments, the linker is a synthetic compound linker (chemical cross-linking agent). Examples of cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

The amino acid sequence of the VH or VL in the minibodies may include modifications such as substitutions, deletions, additions, and/or insertions. For example, the modification may be in one or more of the CDRs (or alternate CDRs) of the anti-αvβ5 antibody or antigen-binding fragment thereof. In certain embodiments, the modification involves one, two, or three amino acid substitutions in one or more CDRs (or alternate CDRs) and/or framework regions of the VH and/or VL domain of the anti-αvβ5 minibody. Such substitutions are made to improve the binding, functional activity, and/or reduce immunogenicity of the anti-αvβ5 minibody. In certain embodiments, the substitutions are conservative amino acid substitutions. In other embodiments, one, two, or three amino acids of the CDRs (or alternate CDRs) of the anti-αvβ5 antibody or antigen-binding fragment thereof may be deleted or added as long as there is αvβ5 binding and/or functional activity when VH and VL are associated. The modified minibodies can inhibit αvβ5 binding to vitronectin; inhibit αvβ5 binding to LAP of TGF-β; and/or inhibit TGF-β signaling.

### Bispecific Antibodies

Bispecific antibodies are antibodies that have binding specificities for at least two different epitopes. Exemplary bispecific antibodies may bind to two different epitopes of the αvβ5 protein. Other such antibodies may combine a αvβ5 binding site with a binding site for another protein (e.g., αvβ1, αvβ3, αvβ6, αvβ8). Bispecific antibodies can be prepared as full length antibodies or low molecular weight forms thereof (e.g., F(ab′)₂ bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Multivalent Antibodies

A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

Conjugated Antibodies

The antibodies disclosed herein may be conjugated antibodies which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, fluorescent substances, luminescent substances, haptens, enzymes, metal chelates, and drugs.

In certain embodiments, an anti-αvβ5 antibody or antigen-binding fragment thereof are modified with a moiety that improves its stabilization and/or retention in circulation, e.g., in blood, serum, or other tissues, e.g., by at least 1.5, 2, 5, 10, or 50 fold. For example, the anti-αvβ5 antibody or antigen-binding fragment thereof can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-αvβ5 antibody or antigen-binding fragment thereof can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) (see, e.g., Chapman et al., Nature Biotechnology, 17: 780-783 (1999), or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides. The efficacy of a therapeutic antibody can be improved by increasing its serum persistence, thereby allowing higher circulating levels, less frequent administration, and reduced doses. The half-life of an IgG depends on its pH-dependent binding to the neonatal receptor FcRn. FcRn, which is expressed on the surface of endothelial cells, binds the IgG in a pH-dependent manner and protects it from degradation. Some antibodies that selectively bind the FcRn at pH 6.0, but not pH 7.4, exhibit a higher half-life in a variety of animal models. In certain embodiments, the antibodies of the present disclosure have one or more mutations at the interface between the CH2 and CH3 domains, such as T250Q/M428L and M252Y/S254T/T256E+H433K/N434F (the numbering is according to the EU index), which increase the binding affinity to FcRn and the half-life of IgG1 in vivo. In other embodiments, the antibodies herein have a modified Fc region comprising at least one modification relative to a wild-type human Fc region, where the modification is selected from the group consisting of 434S, 252Y/428L, 252Y/434S, and 428L/434S, and the numbering is according to the EU index.

The antibodies or antigen-binding fragments thereof can also be conjugated to siRNAs, miRNAs, or anti-miRs to deliver the siRNA, miRNA, or anti-miR to cells expressing αvβ5 (see, e.g., Song et al., Nat. Biotechnol., 23(6):709-17 (2005); Schneider et al., Molecular Therapy Nucleic Acids, 1:e46 (2012)). The siRNAs, miRNAs, miRNA mimics, or anti-miRs can target factors involved in acute kidney injury (e.g., p53). For example, one or more of the following can be targeted to αvβ5-expressing cells using αvβ5 antibodies or antigen-binding fragments thereof conjugated to: an anti-microRNA or siRNA that targets p53, miR-320, miR-16, miR-34a, miR-132, miR-17-3p, miR-362, miR-685, miR-687, miR-207, miR-489, miR-7, miR-132, miR-486, miR-362, miR-467, miR-495, miR-668, miR-694, anti-miR-18a, anti-miR-135b, anti-miR-296, anti-miR-127, anti-miR-322, anti-miR-379, anti-miR-487b, anti-miR-491, anti-miR-324-3p, anti-miR-379, anti-miR-455-3p, and anti-miR-210.

The above-described conjugated antibodies can be prepared by performing chemical modifications on the antibodies or the lower molecular weight forms thereof described herein. Methods for modifying antibodies are well known in the art (e.g., U.S. Pat. No. 5,057,313 and U.S. Pat. No. 5,156,840).

### Methods of Producing Antibodies

The anti-αvβ5 antibodies (or antibody binding fragments thereof) of this disclosure may be produced in bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hanseula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DHSα, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, 293, 293T, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-αvβ5 antibody is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Characterization of the Antibodies

The αvβ5-binding properties of the antibodies described herein may be measured by any standard method, e.g., one or more of the following methods: OCTET®, Surface Plasmon Resonance (SPR), BIACORE™ analysis, Enzyme Linked Immunosorbent Assay (ELISA), EIA (enzyme immunoassay), RIA (radioimmunoassay), and Fluorescence Resonance Energy Transfer (FRET).

The binding interaction of a protein of interest (an anti-αvβ5 antibody) and a target (e.g., β5) can be analyzed using the OCTET® systems. In this method, one of several variations of instruments (e.g., OCTET® QK^{e} and QK), made by the FortéBio company are used to determine protein interactions, binding specificity, and epitope mapping. The OCTET® systems provide an easy way to monitor real-time binding by measuring the changes in polarized light that travels down a custom tip and then back to a sensor.

The binding interaction of a protein of interest (an anti-αvβ5 antibody) and a target (e.g., β5) can be analyzed using Surface Plasmon Resonance (SPR). SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Information from SPR can be used to provide an accurate and quantitative measure of the equilibrium dissociation constant (K_{d}), and kinetic parameters, including Kₒₙ and K_{off}, for the binding of a biomolecule to a target.

Epitopes can also be directly mapped by assessing the ability of different antibodies to compete with each other for binding to human αvβ5 or β5 using BIACORE chromatographic techniques (Pharmacia BIAtechnology Handbook, “Epitope Mapping”, Section 6.3.2, (May 1994); see also Johne et al. (1993) J. Immunol. Methods, 160:191-198).

When employing an enzyme immunoassay, a sample containing an antibody, for example, a culture supernatant of antibody-producing cells or a purified antibody is added to an antigen-coated plate. A secondary antibody labeled with an enzyme such as alkaline phosphatase is added, the plate is incubated, and after washing, an enzyme substrate such as p-nitrophenylphosphate is added, and the absorbance is measured to evaluate the antigen binding activity.

Additional general guidance for evaluating antibodies, e.g., Western blots and immunoprecipitation assays, can be found in Antibodies: A Laboratory Manual, ed. by Harlow and Lane, Cold Spring Harbor press (1988)).

Antibodies with Altered Effector Function

The interaction of antibodies and antibody-antigen complexes with cells of the immune system triggers a variety of responses, referred to herein as effector functions. Immune-mediated effector functions include two major mechanisms: antibody-dependent cell-mediated cytotoxicity (ADCC) and complement-dependent cytotoxicity (CDC). Both of them are mediated by the constant region of the immunoglobulin protein. The antibody Fc domain is, therefore, the portion that defines interactions with immune effector mechanisms.

IgG antibodies activate effector pathways of the immune system by binding to members of the family of cell surface Fcγ receptors and to C1q of the complement system. Ligation of effector proteins by clustered antibodies triggers a variety of responses, including release of inflammatory cytokines, regulation of antigen production, endocytosis, and cell killing. In some clinical applications these responses are crucial for the efficacy of a monoclonal antibody. In others they provoke unwanted side effects such as inflammation and the elimination of antigen-bearing cells. Accordingly, the present invention further relates to αvβ5-binding proteins, including antibodies, with altered, e.g., increased or reduced effector functions.

Effector function of an anti-αvβ5 antibody of the present invention may be determined using one of many known assays. The anti-αvβ5 antibody's effector function may be increased or reduced relative to a second anti-αvβ5 antibody. In some embodiments, the second anti-αvβ5 antibody may be any antibody that binds αvβ5 specifically. In other embodiments, where the anti-αvβ5 antibody of interest has been modified to increase or reduce effector function, the second anti-αvβ5 antibody may be the unmodified or parental version of the antibody.

Effector functions include antibody-dependent cell-mediated cytotoxicity (ADCC), whereby antibodies bind Fc receptors on cytotoxic T cells, natural killer (NK) cells, or macrophages leading to cell death, and complement-dependent cytotoxicity (CDC), which is cell death induced via activation of the complement cascade (reviewed in Daeron, Annu. Rev. Immunol., 15:203-234 (1997); Ward and Ghetie, Therapeutic Immunol., 2:77-94 (1995); and Ravetch and Kinet, Annu. Rev. Immunol. 9:457-492 (1991)). Such effector functions generally require the Fc region to be combined with a binding domain (e.g. an antibody variable domain) and can be assessed using standard assays that are known in the art (see, e.g., WO 05/018572, WO 05/003175, and U.S. Pat. No. 6,242,195).

Effector functions can be avoided by using antibody fragments lacking the Fc domain such as Fab, Fab′2, or single chain Fv. An alternative is to use the IgG4 subtype antibody, which binds to FcγRI but which binds poorly to C1q and FcγRII and RIII. The IgG2 subtype also has reduced binding to Fc receptors, but retains significant binding to the H131 allotype of FcγRIIa and to C1q. Thus, additional changes in the Fc sequence are required to eliminate binding to all the Fc receptors and to C1q.

Several antibody effector functions, including ADCC, are mediated by Fc receptors (FcRs), which bind the Fc region of an antibody. The affinity of an antibody for a particular FcR, and hence the effector activity mediated by the antibody, may be modulated by altering the amino acid sequence and/or post-translational modifications of the Fc and/or constant region of the antibody.

FcRs are defined by their specificity for immunoglobulin isotypes; Fc receptors for IgG antibodies are referred to as FcγR, for IgE as FcεR, for IgA as FcαR and so on. Three subclasses of FcγR have been identified: FcγRI (CD64), FcγRII (CD32) and FcγRIII (CD16). Both FcγRII and FcγRIII have two types: FcγRIIA (CD32) and FcγRIIB (CD32); and FcγRIIIA (CD16a) and FcγRIIIB (CD16b). Because each FcγR subclass is encoded by two or three genes, and alternative RNA splicing leads to multiple transcripts, a broad diversity in FcγR isoforms exists. For example, FcγRII (CD32) includes the isoforms IIa, IIb1, IIb2 IIb3, and IIc.

The binding site on human and murine antibodies for FcγR has been previously mapped to the so-called “lower hinge region” consisting of residues 233-239 (EU index numbering as in Kabat et al., Sequences of Proteins of Immunological Interest, 5th Ed. Public Health Service, National Institutes of Health, Bethesda, Md. (1991), Woof et al., Molec. Immunol. 23:319-330 (1986); Duncan et al., Nature 332:563 (1988); Canfield and Morrison, J Exp. Med. 173:1483-1491 (1991); Chappel et al., Proc. Natl. Acad. Sci USA 88:9036-9040 (1991)). Of residues 233-239, P238 and 5239 are among those cited as possibly being involved in binding. Other previously cited areas possibly involved in binding to FcγR are: G316-K338 (human IgG) for human FcγRI (Woof et al., Mol. Immunol., 23:319-330 (1986)); K274-R301 (human IgG1) for human FcγRIII (Sarmay et al., Molec. Immunol. 21:43-51 (1984)); and Y407-R416 (human IgG) for human FcγRIII (Gergely et al., Biochem. Soc. Trans. 12:739-743 (1984) and Shields et al., J Biol Chem 276: 6591-6604 (2001), Lazar G A et al., Proc Natl Acad Sci 103: 4005-4010 (2006). These and other stretches or regions of amino acid residues involved in FcR binding may be evident to the skilled artisan from an examination of the crystal structures of Ig-FcR complexes (see, e.g., Sondermann et al. 2000 Nature 406(6793):267-73 and Sondermann et al. 2002 Biochem Soc Trans. 30(4):481-6). Accordingly, the anti-αvβ5 antibodies of the present invention include modifications of one or more of the aforementioned residues (to increase or decrease effector function as needed).

Another approach for altering monoclonal antibody effector function include mutating amino acids on the surface of the monoclonal antibody that are involved in effector binding interactions (Lund, J., et al. (1991) J. Immunol. 147(8): 2657-62; Shields, R. L. et al. (2001) J Biol. Chem. 276(9): 6591-604).

Methods of increasing effector function of antibodies are well known in the art (see, e.g., Kelley et al., Methods Mol. Biol., 901:277-93 (2012); Natsume et al., Drug Des Devel Ther., 3:7-16 (2009); U.S. Pat. No. 8,188,231, U.S. Pat. No. 7,960,512). In one embodiment, the αvβ5 antibodies have one, two, three, four, five, six, seven, or more amino acid substitutions at a position selected from the group consisting of 221, 222, 223, 224, 225, 227, 228, 230, 231, 232, 233, 234, 235, 236, 237, 238, 239, 240, 241, 243, 244, 245, 246, 247, 249, 255, 258, 260, 262, 263, 264, 265, 266, 267, 268, 269, 270, 271, 272, 273, 274, 275, 276, 278, 280, 281, 282, 283, 284, 285, 286, 288, 290, 291, 292, 293, 294, 295, 296, 297, 298, 299, 300, 301, 302, 303, 304, 305, 313, 317, 318, 320, 322, 323, 324, 325, 326, 327, 328, 329, 330, 331, 332, 333, 334, 335, 336, and 337, wherein the numbering of the residues in the Fc region is that of the EU index as in Kabat. In certain embodiments, the αvβ5 antibodies have one, two, three, four, five, six, seven, or more of the amino acid substitutions selected from the group consisting of: D221K, D221Y, K222E, K222Y, T223E, T223K, H224E, H224Y, T225E, T225K, T225W, P227E, P227G, P227K, P227Y, P228E, P228G, P228K, P228Y, P230A, P230E, P230G, P230Y, A231E, A231G, A231K, A231P, A231Y, P232E, P232G, P232K, P232Y, E233A, E233D, E233F, E233G, E233H, E233I, E233K, E233L, E233M, E233N, E233Q, E233R, E233S, E233T, E233V, E233W, E233Y, L234A, L234D, L234E, L234F, L234G, L234H, L234I, L234K, L234M, L234N, L234P, L234Q, L234R, L234S, L234T, L234V, L234W, L234Y, L235A, L235D, L235E, L235F, L235G, L235H, L235I, L235K, L235M, L235N, L235P, L235Q, L235R, L235S, L235T, L235V, L235W, L235Y, G236A, G236D, G236E, G236F, G236H, G236I, G236K, G236L, G236M, G236N, G236P, G236Q, G236R, G236S, G236T, G236V, G236W, G236Y, G237D, G237E, G237F, G237H, G237I, G237K, G237L, G237M, G237N, G237P, G237Q, G237R, G237S, G237T, G237V, G237W, G237Y, P238D, P238E, P238F, P238G, P238H, P238I, P238K, P238L, P238M, P238N, P238Q, P238R, P238S, P238T, P238V, P238W, P238Y, S239D, S239E, S239F, S239G, S239H, S239I, S239K, S239L, S239M, S239N, S239P, S239Q, S239R, S239T, S239V, S239W, S239Y, V240A, V240I, V240M, V240T, F241D, F241E, F241L, F241R, F241S, F241W, F241Y, F243E, F243H, F243L, F243Q, F243R, F243W, F243Y, P244H, P245A, K246D, K246E, K246H, K246Y, P247G, P247V, D249H, D249Q, D249Y, R255E, R255Y, E258H, E258S, E258Y, T260D, T260E, T260H, T260Y, V262A, V262E, V262F, V262I, V262T, V263A, V263I, V263M, V263T, V264A, V264D, V264E, V264F, V264G, V264H, V264I, V264K, V264L, V264M, V264N, V264P, V264Q, V264R, V264S, V264T, V264W, V264Y, D265F, D265G, D265H, D265I, D265K, D265L, D265M, D265N, D265P, D265Q, D265R, D265S, D265T, D265V, D265W, D265Y, V266A, V266I, V266M, V266T, S267D, S267E, S267F, S267H, S267I, S267K, S267L, S267M, S267N, S267P, S267Q, S267R, S267T, S267V, S267W, S267Y, H268D, H268E, H268F, H268G, H268I, H268K, H268L, H268M, H268P, H268Q, H268R, H268T, H268V, H268W, E269F, E269G, E269H, E269I, E269K, E269L, E269M, E269N, E269P, E269R, E269S, E269T, E269V, E269W, E269Y, D270F, D270G, D270H, D270I, D270L, D270M, D270P, D270Q, D270R, D270S, D270T, D270W, D270Y, P271A, P271D, P271E, P271F, P271G, P271H, P271I, P271K, P271L, P271M, P271N, P271Q, P271R, P271S, P271T, P271V, P271W, P271Y, E272D, E272F, E272G, E272H, E272I, E272K, E272L, E272M, E272P, E272R, E272S, E272T, E272V, E272W, E272Y, V273I, K274D, K274E, K274F, K274G, K274H, K274I, K274L, K274M, K274N, K274P, K274R, K274T, K274V, K274W, K274Y, F275L, F275W, N276D, N276E, N276F, N276G, N276H, N276I, N276L, N276M, N276P, N276R, N276S, N276T, N276V, N276W, N276Y, Y278D, Y278E, Y278G, Y278H, Y278I, Y278K, Y278L, Y278M, Y278N, Y278P, Y278Q, Y278R, Y278S, Y278T, Y278V, Y278W, D280G, D280K, D280L, D280P, D280W, G281D, G281E, G281K, G281N, G281P, G281Q, G281Y, V282E, V282G, V282K, V282P, V282Y, E283G, E283H, E283K, E283L, E283P, E283R, E283Y, V284D, V284E, V284L, V284N, V284Q, V284T, V284Y, H285D, H285E, H285K, H285Q, H285W, H285Y, N286E, N286G, N286P, N286Y, K288D, K288E, K288Y, K290D, K290H, K290L, K290N, K290W, P291D, P291E, P291G, P291H, P291I, P291Q, P291T, R292D, R292E, R292T, R292Y, E293F, E293G, E293H, E293I, E293L, E293M, E293N, E293P, E293R, E293S, E293T, E293V, E293W, E293Y, E294F, E294G, E294H, E294I, E294K, E294L, E294M, E294P, E294R, E294S, E294T, E294V, E294W, E294Y, Q295D, Q295E, Q295F, Q295G, Q295H, Q295I, Q295M, Q295N, Q295P, Q295R, Q295S, Q295T, Q295V, Q295W, Q295Y, Y296A, Y296D, Y296E, Y296G, Y296H, Y296I, Y296K, Y296L, Y296M, Y296N, Y296Q, Y296R, Y296S, Y296T, Y296V, N297D, N297E, N297F, N297G, N297H, N297I, N297K, N297L, N297M, N297P, N297Q, N297R, N297S, N297T, N297V, N297W, N297Y, S298D, S298E, S298F, S298H, S298I, S298K, S298M, S298N, S298Q, S298R, S298T, S298W, S298Y, T299A, T299D, T299E, T299F, T299G, T299H, T299I, T299K, T299L, T299M, T299N, T299P, T299Q, T299R, T299S, T299V, T299W, T299Y, Y300A, Y300D, Y300E, Y300G, Y300H, Y300K, Y300M, Y300N, Y300P, Y300Q, Y300R, Y300S, Y300T, Y300V, Y300W, R301D, R301E, R301H, R301Y, V302I, V303D, V303E, V303Y, S304D, S304H, S304L, S304N, S304T, V305E, V305T, V305Y, W313F, K317E, K317Q, E318H, E318L, E318Q, E318R, E318Y, K320D, K320F, K320G, K320H, K320I, K320L, K320N, K320P, K320S, K320T, K320V, K320W, K320Y, K322D, K322F, K322G, K322H, K322I, K322P, K322S, K322T, K322V, K322W, K322Y, V323I, S324D, S324F, S324G, S324H, S324I, S324L, S324M, S324P, S324R, S324T, S324V, S324W, S324Y, N325A, N325D, N325E, N325F, N325G, N325H, N325I, N325K, N325L, N325M, N325P, N325Q, N325R, N325S, N325T, N325V, N325W, N325Y, K326I, K326L, K326P, K326T, A327D, A327E, A327F, A327H, A327I, A327K, A327L, A327M, A327N, A327P, A327R, A327S, A327T, A327V, A327W, A327Y, L328A, L328D, L328E, L328F, L328G, L328H, L328I, L328K, L328M, L328N, L328P, L328Q, L328R, L328S, L328T, L328V, L328W, L328Y, P329D, P329E, P329F, P329G, P329H, P329I, P329K, P329L, P329M, P329N, P329Q, P329R, P329S, P329T, P329V, P329W, P329Y, A330E, A330F, A330G, A330H, A330I, A330L, A330M, A330N, A330P, A330R, A330S, A330T, A330V, A330W, A330Y, P331D, P331F, P331H, P331I, P331L, P331M, P331Q, P331R, P331T, P331V, P331W, P331Y, I332A, I332D, I332E, I332F, I332H, I332K, I332L, I332M, I332N, I332P, I332Q, I332R, I332S, I332T, I332V, I332W, I332Y, E333F, E333H, E333I, E333L, E333M, E333P, E333T, E333Y, K334F, K334I, K334L, K334P, K334T, T335D, T335F, T335G, T335H, T335I, T335L, T335M, T335N, T335P, T335R, T335S, T335V, T335W, T335Y, I336E, I336K, I336Y, S337E, S337H, and S337N, wherein the numbering of the residues in the Fc region is that of the EU index as in Kabat. In a particular embodiment, the αvβ5 antibodies comprise one, two, or three of the following mutations: S239D, S239D/I332E, S239D/I332E/A330L, S239D/I332E/G236A, S298A, A330L I332E, E333A, and K334A.

The presence of oligosaccharides—specifically, the N-linked oligosaccharide at asparigine-297 in the CH2 domain of IgG1—is important for binding to FcγR as well as C1q. Reducing the fucose content of antibodies improves effector function (see, e.g., U.S. Pat. No. 8,163,551). In certain embodiments the αvβ5 antibodies have reduced fucosylation and amino acid substitutions that increase effector function (e.g., one, two, or three of the following mutations: S298A; E333A, and K334A). Effector function can also be achieved by preparing and expressing the anti-αvβ5 antibodies described herein in the presence of alpha-mannosidase I inhibitors (e.g., kifunensine) at a concentration of the inhibitor of about 60-200 ng/mL (e.g., 60 ng/mL, 75 ng/mL, 100 ng/mL, 150 ng/ml). Antibodies expressed in the presence of alpha-mannosidase I inhibitors contain mainly oligomannose-type glycans and generally demonstrate increased ADCC activity and affinity for FcγRIIIA, but reduced C1q binding.

Anti-αvβ5 antibodies of the present disclosure with increased effector function include antibodies with increased binding affinity for one or more Fc receptors (FcRs) relative to a parent or non-variant anti-αvβ5 antibody. Accordingly, anti-αvβ5 antibodies with increased FcR binding affinity includes anti-αvβ5 antibodies that exhibit a 1.5-fold, 2-fold, 2.5-fold, 3-fold, 4-fold, or 5-fold or higher increase in binding affinity to one or more Fc receptors compared to a parent or non-variant anti-αvβ5 antibody. In some embodiments, an anti-αvβ5 antibody with increased effector function binds to an FcR with about 10-fold greater affinity relative to a parent or non-variant antibody. In other embodiments, an anti-αvβ5 antibody with increased effector function binds to an FcR with about 15-fold greater affinity or with about 20-fold greater affinity relative to a parent or non-variant antibody. The FcR receptor may be one or more of FcγRI (CD64), FcγRII (CD32), and FcγRIII, and isoforms thereof, and FcεR, FcμR, FcδR, and/or an FcαR. In particular embodiments, an anti-αvβ5 antibody with increased effector function exhibits a 1.5-fold, 2-fold, 2.5-fold, 3-fold, 4-fold, or 5-fold or higher increase in binding affinity to FcγRIIa.

To reduce effector function, one can use combinations of different subtype sequence segments (e.g., IgG2 and IgG4 combinations) to give a greater reduction in binding to Fcγ receptors than either subtype alone (Armour et al., Eur. J. Immunol., 29:2613-1624 (1999); Mol. Immunol., 40:585-593 (2003)). In addition, sites of N-linked glycosylation can be removed as a means of reducing effector function. A large number of Fc variants having altered and/or reduced affinities for some or all Fc receptor subtypes (and thus for effector functions) are known in the art. See, e.g., US 2007/0224188; US 2007/0148171; US 2007/0048300; US 2007/0041966; US 2007/0009523; US 2007/0036799; US 2006/0275283; US 2006/0235208; US 2006/0193856; US 2006/0160996; US 2006/0134105; US 2006/0024298; US 2005/0244403; US 2005/0233382; US 2005/0215768; US 2005/0118174; US 2005/0054832; US 2004/0228856; US 2004/132101; US 2003/158389; see also U.S. Pat. Nos. 7,183,387; 6,737,056; 6,538,124; 6,528,624; 6,194,551; 5,624,821; 5,648,260. In certain embodiments amino acids at positions 232, 234, 235, 236, 237, 239, 264, 265, 267, 269, 270, 299, 325, 328, 329, and 330 (numbered according to Kabat) are substituted to reduce effector function. Non-limiting examples of substitutions that reduce effector function include one or more of: K322A; L234A/L235A; G236T; G236R; G236Q; H268A; H268Q; V309L; A330S; P331S; V234A/G237A/P238S/H268A/V309L/A330S/P331S; E233P/L234V/L235A/G236Q+A327G/A330S/P331S; and L235E+E318A/K320A/K322A.

Anti-αvβ5 antibodies of the present invention with reduced effector function include antibodies with reduced binding affinity for one or more Fc receptors (FcRs) relative to a parent or non-variant anti-αvβ5 antibody. Accordingly, anti-αvβ5 antibodies with reduced FcR binding affinity includes anti-αvβ5 antibodies that exhibit a 1.5-fold, 2-fold, 2.5-fold, 3-fold, 4-fold, or 5-fold or higher decrease in binding affinity to one or more Fc receptors compared to a parent or non-variant anti-αvβ5 antibody. In some embodiments, an anti-αvβ5 antibody with reduced effector function binds to an FcR with about 10-fold less affinity relative to a parent or non-variant antibody. In other embodiments, an anti-αvβ5 antibody with reduced effector function binds to an FcR with about 15-fold less affinity or with about 20-fold less affinity relative to a parent or non-variant antibody. The FcR receptor may be one or more of FcγRI (CD64), FcγRII (CD32), and FcγRIII, and isoforms thereof, and FcεR, FcμR, FcδR, and/or an FcαR. In particular embodiments, an anti-αvβ5 antibody with reduced effector function exhibits a 1.5-fold, 2-fold, 2.5-fold, 3-fold, 4-fold, or 5-fold or higher decrease in binding affinity to FcγRIIa.

In CDC, the antibody-antigen complex binds complement, resulting in the activation of the complement cascade and generation of the membrane attack complex. Activation of the classical complement pathway is initiated by the binding of the first component of the complement system (C1q) to antibodies (of the appropriate subclass) which are bound to their cognate antigen; thus the activation of the complement cascade is regulated in part by the binding affinity of the immunoglobulin to C1q protein. To activate the complement cascade, it is necessary for C1q to bind to at least two molecules of IgG1, IgG2, or IgG3, but only one molecule of IgM, attached to the antigenic target (Ward and Ghetie, Therapeutic Immunology 2:77-94 (1995) p. 80). To assess complement activation, a CDC assay, e.g. as described in Gazzano-Santoro et al., J. Immunol. Methods, 202:163 (1996), may be performed.

Various residues of the IgG molecule are involved in binding to C1q including the Glu318, Lys320 and Lys322 residues on the CH2 domain, amino acid residue 331 located on a turn in close proximity to the same beta strand, the Lys235 and Gly237 residues located in the lower hinge region, and residues 231 to 238 located in the N-terminal region of the CH2 domain (see e.g., Xu et al., J. Immunol. 150:152A (Abstract) (1993), WO94/29351; Tao et al, J. Exp. Med., 178:661-667 (1993); Brekke et al., Eur. J. Immunol., 24:2542-47 (1994); Burton et al; Nature, 288:338-344 (1980); Duncan and Winter, Nature 332:738-40 (1988); Idusogie et al J Immunol 164: 4178-4184 (2000; U.S. Pat. No. 5,648,260, and U.S. Pat. No. 5,624,821).

Anti-αvβ5 antibodies with improved C1q binding can comprise an amino acid substitution at one, two, three, or four of amino acid positions 326, 327, 333 and 334 of the human IgG Fc region, where the numbering of the residues in the IgG Fc region is that of the EU index as in Kabat. In one embodiment, the anti-αvβ5 antibodies include the following amino acid substitutions: K326W/E333S, which are known to increase binding of an IgG1 antibody to C1q (Steurer W. et al., J Immunol., 155(3):1165-74 (1995)).

Anti-αvβ5 antibodies with reduced C1q binding can comprise an amino acid substitution at one, two, three, or four of amino acid positions 270, 322, 329 and 331 of the human IgG Fc region, where the numbering of the residues in the IgG Fc region is that of the EU index as in Kabat. As an example in IgG1, two mutations in the COOH terminal region of the CH2 domain of human IgG1—K322A and P329A—do not activate the CDC pathway and were shown to result in more than a 100 fold decrease in C1q binding (U.S. Pat. No. 6,242,195).

Accordingly, in certain embodiments, an anti-αvβ5 antibody of the present invention exhibits increased or reduced binding to a complement protein relative to a second anti-αvβ5 antibody. In certain embodiments, an anti-αvβ5 antibody of the invention exhibits increased or reduced binding to C1q by a factor of about 1.5-fold or more, about 2-fold or more, about 3-fold or more, about 4-fold or more, about 5-fold or more, about 6-fold or more, about 7-fold or more, about 8-fold or more, about 9-fold or more, about 10-fold or more, or about 15-fold or more, relative to a second anti-αvβ5 antibody.

Thus, in certain embodiments of the invention, one or more of these residues may be modified, substituted, or removed or one or more amino acid residues may be inserted so as to increase or decrease CDC activity of the anti-αvβ5 antibodies provided herein.

In certain other embodiments, the present invention provides an anti-αvβ5 antibody that exhibits reduced binding to one or more FcR receptors but that maintains its ability to bind complement (e.g., to a similar or, in some embodiments, to a lesser extent than a native, non-variant, or parent anti-αvβ5 antibody). Accordingly, an anti-αvβ5 antibody of the present invention may bind and activate complement while exhibiting reduced binding to an FcR, such as, for example, FcγRIIa (e.g., FcγRIIa expressed on platelets). Such an antibody with reduced or no binding to FcγRIIa (such as FcγRIIa expressed on platelets, for example) but that can bind C1q and activate the complement cascade to at least some degree will reduce the risk of thromboembolic events while maintaining perhaps desirable effector functions. In alternative embodiments, an anti-αvβ5 antibody of the present invention exhibits reduced binding to one or more FcRs but maintains its ability to bind one or more other FcRs. See, for example, US 2007-0009523, 2006-0194290, 2005-0233382, 2004-0228856, and 2004-0191244, which describe various amino acid modifications that generate antibodies with reduced binding to FcRI, FcRII, and/or FcRIII, as well as amino acid substitutions that result in increased binding to one FcR but decreased binding to another FcR.

Accordingly, effector functions involving the constant region of an anti-αvβ5 antibody may be modulated by altering properties of the constant region, and the Fc region in particular. In certain embodiments, the anti-αvβ5 antibody having increased or decreased effector function is compared with a second antibody with effector function and which may be a non-variant, native, or parent antibody comprising a native constant or Fc region that mediates effector function.

A native sequence Fc or constant region comprises an amino acid sequence identical to the amino acid sequence of a Fc or constant chain region found in nature. Preferably, a control molecule used to assess relative effector function comprises the same type/subtype Fc region as does the test or variant antibody. A variant or altered Fc or constant region comprises an amino acid sequence which differs from that of a native sequence heavy chain region by virtue of at least one amino acid modification (such as, for example, post-translational modification, amino acid substitution, insertion, or deletion). Accordingly, the variant constant region may contain one or more amino acid substitutions, deletions, or insertions that results in altered post-translational modifications, including, for example, an altered glycosylation pattern. A parent antibody or Fc region is, for example, a variant having normal effector function used to construct a constant region (i.e., Fc) having altered, e.g., increased effector function.

Antibodies with altered (e.g., increased) effector function(s) may be generated by engineering or producing antibodies with variant constant, Fc, or heavy chain regions. Recombinant DNA technology and/or cell culture and expression conditions may be used to produce antibodies with altered function and/or activity. For example, recombinant DNA technology may be used to engineer one or more amino acid substitutions, deletions, or insertions in regions (such as, for example, Fc or constant regions) that affect antibody function including effector functions. Alternatively, changes in post-translational modifications, such as, e.g. glycosylation patterns, may be achieved by manipulating the host cell and cell culture and expression conditions by which the antibody is produced.

Certain embodiments of the present invention relate to an anti-αvβ5 antibody comprising one or more (i.e., 1, 2 or 3) heavy chain CDR sequences selected from VH CDR1 of SEQ ID NO:3, VH CDR2 of SEQ ID NO:4, and VH CDR3 of SEQ ID NO:5; or one or more (i.e., 1, 2 or 3) heavy chain alternate CDR sequences selected from: VH CDR1 of SEQ ID NO:21, VH CDR2 of SEQ ID NO:24, and VH CDR3 of SEQ ID NO:5; or VH CDR1 of SEQ ID NO:22, VH CDR2 of SEQ ID NO:25, and VH CDR3 of SEQ ID NO:5; or VH CDR1 of SEQ ID NO:23, VH CDR2 of SEQ ID NO:26, and VH CDR3 of SEQ ID NO:7, wherein the antibody further comprises a variant Fc region that confers increased or reduced effector function compared to a native or parental Fc region. In further embodiments, the anti-αvβ5 antibody comprises at least two of the CDRs (or alternate CDRs), and in other embodiments the antibody comprises all three of the heavy chain CDR (or alternate CDR) sequences. These anti-αvβ5 antibodies inhibit the interaction between αvβ5 and vitronectin, inhibit the interaction between αvβ5 and LAP of TGF-β, inhibit TGF-β signaling, inhibit TGF-β activation, and/or inhibit the interaction between αvβ5 and its RGD-motif containing ligands.

Other embodiments of the present invention relate to an anti-αvβ5 antibody comprising one or more (i.e., 1, 2 or 3) light chain CDR sequences selected from VL CDR1 of SEQ ID NO:6, VL CDR2 of SEQ ID NO:7, and VL CDR3 of SEQ ID NO:8; or one or more (i.e., 1, 2 or 3) light chain alternate CDR sequences selected from VL CDR1 of SEQ ID NO:28, VL CDR2 of SEQ ID NO:29, and VL CDR3 of SEQ ID NO:30, the antibody further comprising a variant Fc region that confers increased or reduced effector function compared to a native or parental Fc region. In further embodiments, the anti-αvβ5 antibody comprises at least two of the light chain CDRs (or alternate CDRs), and in other embodiments the antibody comprises all three of the light chain CDR (or alternate CDR) sequences. These anti-αvβ5 antibodies inhibit the interaction between αvβ5 and vitronectin, inhibit the interaction between αvβ5 and LAP of TGF-β, inhibit TGF-β signaling, inhibit TGF-β activation, and/or inhibit the interaction between αvβ5 and its RGD-motif containing ligands.

In further embodiments of the present invention, the anti-αvβ5 antibody with increased or reduced effector function comprises all three light chain CDR sequences (CDRs 1, 2, and 3) or all three light chain alternate CDRs of SEQ ID NO:11 and comprises all three heavy chain CDR sequences (CDRs 1, 2, and 3) or all three heavy chain alternate CDRs of SEQ ID NO:9. In certain embodiments, the anti-αvβ5 antibody with increased or reduced effector function comprises: three or fewer, two or fewer, or one amino acid substitution in one, two, or three CDRs (or alternate CDRs) of SEQ ID NO:9 and three or fewer, two or fewer, or one amino acid substitution in one, two, or three CDRs (or alternate CDRs) of SEQ ID NO:11. These anti-αvβ5 antibodies inhibit the interaction between αvβ5 and vitronectin, inhibit the interaction between αvβ5 and LAP of TGF-β, inhibit TGF-β signaling, inhibit TGF-β activation, and/or inhibit the interaction between αvβ5 and its RGD-motif containing ligands.

Anti-αvβ5 Antibodies with Altered Glycosylation

Glycan removal produces a structural change that should greatly reduce binding to all members of the Fc receptor family across species. In glycosylated antibodies, including anti-αvβ5 antibodies, the glycans (oligosaccharides) attached to the conserved N-linked site in the CH2 domains of the Fc dimer are enclosed between the CH2 domains, with the sugar residues making contact with specific amino acid residues on the opposing CH2 domain. Different glycosylation patterns are associated with different biological properties of antibodies (Jefferis and Lund, 1997, Chem. Immunol., 65: 111-128; Wright and Morrison, 1997, Trends Biotechnol., 15: 26-32). Certain specific glycoforms confer potentially advantageous biological properties. Loss of the glycans changes spacing between the domains and increases their mobility relative to each other and is expected to have an inhibitory effect on the binding of all members of the Fc receptor family. For example, in vitro studies with various glycosylated antibodies have demonstrated that removal of the CH2 glycans alters the Fc structure such that antibody binding to Fc receptors and the complement protein C1Q are greatly reduced. Another known approach to reducing effector functions is to inhibit production of or remove the N-linked glycans at position 297 (EU numbering) in the CH2 domain of the Fc (Nose et al., 1983 PNAS 80: 6632; Leatherbarrow et al., 1985 Mol. Immunol. 22: 407; Tao et al., 1989 J. Immunol. 143: 2595; Lund et al., 1990 Mol. Immunol. 27: 1145; Dorai et al., 1991 Hybridoma 10:211; Hand et al., 1992 Cancer Immunol. Immunother. 35:165; Leader et al., 1991 Immunology 72: 481; Pound et al., 1993 Mol. Immunol. 30:233; Boyd et al., 1995 Mol. Immunol. 32: 1311). It is also known that different glycoforms can profoundly affect the properties of a therapeutic, including pharmacokinetics, pharmacodynamics, receptor-interaction and tissue-specific targeting (Graddis et al., 2002, Curr Pharm Biotechnol. 3: 285-297). In particular, for antibodies, the oligosaccharide structure can affect properties relevant to protease resistance, the serum half-life of the antibody mediated by the FcRn receptor, phagocytosis and antibody feedback, in addition to effector functions of the antibody (e.g., binding to the complement complex C1, which induces CDC, and binding to FcγR receptors, which are responsible for modulating the ADCC pathway) (Nose and Wigzell, 1983; Leatherbarrow and Dwek, 1983; Leatherbarrow et al., 1985; Walker et al., 1989; Carter et al., 1992, PNAS, 89: 4285-4289).

Accordingly, another means of modulating effector function of antibodies includes altering glycosylation of the antibody constant region. Altered glycosylation includes, for example, a decrease or increase in the number of glycosylated residues, a change in the pattern or location of glycosylated residues, as well as a change in sugar structure(s). The oligosaccharides found on human IgGs affects their degree of effector function (Raju, T. S. BioProcess International April 2003. 44-53); the microheterogeneity of human IgG oligosaccharides can affect biological functions such as CDC and ADCC, binding to various Fc receptors, and binding to C1q protein (Wright A. & Morrison SL. TIBTECH 1997, 15 26-32; Shields et al. J Biol Chem. 2001 276(9):6591-604; Shields et al. J Biol Chem. 2002; 277(30):26733-40; Shinkawa et al. J Biol Chem. 2003 278(5):3466-73; Umana et al. Nat Biotechnol. 1999 February; 17(2): 176-80). For example, the ability of IgG to bind C1q and activate the complement cascade may depend on the presence, absence or modification of the carbohydrate moiety positioned between the two CH2 domains (which is normally anchored at Asn297) (Ward and Ghetie, Therapeutic Immunology 2:77-94 (1995).

Glycosylation sites in an Fc-containing polypeptide, for example an antibody such as an IgG antibody, may be identified by standard techniques. The identification of the glycosylation site can be experimental or based on sequence analysis or modeling data. Consensus motifs, that is, the amino acid sequence recognized by various glycosyl transferases, have been described. For example, the consensus motif for an N-linked glycosylation motif is frequently NXT or NXS, where X can be any amino acid except proline. Several algorithms for locating a potential glycosylation motif have also been described. Accordingly, to identify potential glycosylation sites within an antibody or Fc-containing fragment, the sequence of the antibody is examined, for example, by using publicly available databases such as the website provided by the Center for Biological Sequence Analysis (see NetNGlyc services for predicting N-linked glycosylation sites and NetOGlyc services for predicting O-linked glycosylation sites).

In vivo studies have confirmed the reduction in the effector function of aglycosyl antibodies. For example, an aglycosyl anti-CD8 antibody is incapable of depleting CD8-bearing cells in mice (Isaacs, 1992 J. Immunol. 148: 3062) and an aglycosyl anti-CD3 antibody does not induce cytokine release syndrome in mice or humans (Boyd, 1995 supra; Friend, 1999 Transplantation 68:1632).

Importantly, while removal of the glycans in the CH2 domain appears to have a significant effect on effector function, other functional and physical properties of the antibody remains unaltered. Specifically, it has been shown that removal of the glycans had little to no effect on serum half-life and binding to antigen (Nose, 1983 supra; Tao, 1989 supra; Dorai, 1991 supra; Hand, 1992 supra; Hobbs, 1992 Mol. Immunol. 29:949).

Although there is in vivo validation of the aglycosyl approach, there are reports of residual effector function with aglycosyl mAbs (see, e.g., Pound, J. D. et al. (1993) Mol. Immunol. 30(3): 233-41; Dorai, H. et al. (1991) Hybridoma 10(2): 211-7). Armour et al. show residual binding to FcγRIIa and FcγRIIb proteins (Eur. J. Immunol. (1999) 29: 2613-1624; Mol. Immunol. 40 (2003) 585-593). Thus a further decrease in effector function, particularly complement activation, may be important to guarantee complete ablation of activity in some instances. For that reason, aglycosyl forms of IgG2 and IgG4 and a G1/G4 hybrid are envisioned as being useful in methods and antibody compositions of the invention having reduced effector functions.

The anti-αvβ5 antibodies of the present invention may be modified or altered to elicit reduced effector function(s) (compared to a second αvβ5-specific antibody) while optionally retaining the other valuable attributes of the Fc portion.

Accordingly, in certain embodiments, the present invention relates to aglycosyl anti-αvβ5 antibodies with decreased effector function, which are characterized by a modification at the conserved N-linked site in the CH2 domains of the Fc portion of the antibody. A modification of the conserved N-linked site in the CH2 domains of the Fc dimer can lead to aglycosyl anti-αvβ5 antibodies. Examples of such modifications include mutation of the conserved N-linked site in the CH2 domains of the Fc dimer, removal of glycans attached to the N-linked site in the CH2 domains, and prevention of glycosylation. For example, an aglycosyl anti-αvβ5 antibody may be created by changing the canonical N-linked Asn site in the heavy chain CH2 domain to a Gln residue (see, for example, WO 05/03175 and US 2006-0193856).

In one embodiment of present invention, the modification comprises a mutation at the heavy chain glycosylation site to prevent glycosylation at the site. Thus, in one embodiment of this invention, the aglycosyl anti-αvβ5 antibodies are prepared by mutation of the heavy chain glycosylation site, i.e., mutation of N298Q (N297 using Kabat EU numbering) and expressed in an appropriate host cell. For example, this mutation may be accomplished by following the manufacturer's recommended protocol for unique site mutagenesis kit from Amersham-Pharmacia Biotech® (Piscataway, N.J., USA).

The mutated antibody can be stably expressed in a host cell (e. g. NSO or CHO cell) and then purified. As one example, purification can be carried out using Protein A and gel filtration chromatography. It will be apparent to those of skill in the art that additional methods of expression and purification may also be used.

In another embodiment of the present invention, the aglycosyl anti-αvβ5 antibodies have decreased effector function, wherein the modification at the conserved N-linked site in the CH2 domains of the Fc portion of said antibody or antibody derivative comprises the removal of the CH2 domain glycans, i.e., deglycosylation. These aglycosyl anti-αvβ5 antibodies may be generated by conventional methods and then deglycosylated enzymatically. Methods for enzymatic deglycosylation of antibodies are well known to those of skill in the art (Williams, 1973; Winkelhake & Nicolson, 1976 J. Biol Chem. 251:1074-80.).

In another embodiment of this invention, deglycosylation may be achieved by growing host cells which produce the antibodies in culture medium comprising a glycosylation inhibitor such as tunicamycin (Nose & Wigzell, 1983). That is, the modification is the reduction or prevention of glycosylation at the conserved N-linked site in the CH2 domains of the Fc portion of said antibody.

In other embodiments of this invention, recombinant X polypeptides (or cells or cell membranes containing such polypeptides) may be used as an antigen to generate an anti-αvβ5 antibody or antibody derivatives, which may then be deglycosylated.

In alternative embodiments, agyclosyl anti-αvβ5 antibodies or anti-αvβ5 antibodies with reduced glycosylation may be produced by the method described in Taylor et al. (WO 05/18572 and US 2007-0048300). For example, in one embodiment, an anti-αvβ5 aglycosyl antibody may be produced by altering a first amino acid residue (e.g., by substitution, insertion, deletion, or by chemical modification), wherein the altered first amino acid residue inhibits the glycosylation of a second residue by either steric hindrance or charge or both. In certain embodiments, the first amino acid residue is modified by amino acid substitution. In further embodiments, the amino acid substitution is selected from the group consisting of Gly, Ala, Val, Leu, Ile, Phe, Asn, Gln, Trp, Pro, Ser, Thr, Tyr, Cys, Met, Asp, Glu, Lys, Arg, and His. In other embodiments, the amino acid substitution is a non-traditional amino acid residue. The second amino acid residue may be near or within a glycosylation motif, for example, an N-linked glycosylation motif that contains the amino acid sequence NXT or NXS. In one exemplary embodiment, the first amino acid residue is amino acid 299 and the second amino acid residue is amino acid 297, according to the Kabat numbering. For example, the first amino acid substitution may be T299A, T299N, T299G, T299Y, T299C, T299H, T299E, T299D, T299K, T299R, T299G, T299I, T299L, T299M, T299F, T299P, T299W, and T299V, according to the Kabat numbering. In particular embodiments, the amino acid substitution is T299C.

Effector function may also be reduced by modifying an antibody of the present invention such that the antibody contains a blocking moiety. Exemplary blocking moieties include moieties of sufficient steric bulk and/or charge such that reduced glycosylation occurs, for example, by blocking the ability of a glycosidase to glycosylate the polypeptide. The blocking moiety may additionally or alternatively reduce effector function, for example, by inhibiting the ability of the Fc region to bind a receptor or complement protein. In some embodiments, the present invention relates to an αvβ5-binding protein, e.g., an anti-αvβ5 antibody, comprising a variant Fc region, the variant Fc region comprising a first amino acid residue and an N-glycosylation site, the first amino acid residue modified with side chain chemistry to achieve increased steric bulk or increased electrostatic charge compared to the unmodified first amino acid residue, thereby reducing the level of or otherwise altering glycosylation at the N-glycosylation site. In certain of these embodiments, the variant Fc region confers reduced effector function compared to a control, non-variant Fc region. In further embodiments, the side chain with increased steric bulk is a side chain of an amino acid residue selected from the group consisting of Phe, Trp, His, Glu, Gln, Arg, Lys, Met and Tyr. In yet further embodiments, the side chain chemistry with increased electrostatic charge is a side chain of an amino acid residue selected from the group consisting of Asp, Glu, Lys, Arg, and His.

Accordingly, in one embodiment, glycosylation and Fc binding can be modulated by substituting T299 with a charged side chain chemistry such as D, E, K, or R. The resulting antibody will have reduced glycosylation as well as reduced Fc binding affinity to an Fc receptor due to unfavorable electrostatic interactions.

In another embodiment, a T299C variant antibody, which is both aglycosylated and capable of forming a cysteine adduct, may exhibit less effector function (e.g., FcγRI binding) compared to its aglycosylated antibody counterpart (see, e.g., WO 05/18572). Accordingly, alteration of a first amino acid proximal to a glycosylation motif can inhibit the glycosylation of the antibody at a second amino acid residue; when the first amino acid is a cysteine residue, the antibody may exhibit even further reduced effector function. In addition, inhibition of glycosylation of an antibody of the IgG4 subtype may have a more profound effect on FcγRI binding compared to the effects of agycosylation in the other subtypes.

In additional embodiments, the present invention relates to anti-αvβ5 antibodies with altered glycosylation that exhibit reduced binding to one or more FcR receptors and that optionally also exhibit increased or normal binding to one or more Fc receptors and/or complement—e.g., antibodies with altered glycosylation that at least maintain the same or similar binding affinity to one or more Fc receptors and/or complement as a native, control anti-αvβ5 antibody). For example, anti-αvβ5 antibodies with predominantly Man₅GlcNAc₂N-glycan as the glycan structure present (e.g., wherein Man₅GlcNAc₂N-glycan structure is present at a level that is at least about 5 mole percent more than the next predominant glycan structure of the Ig composition) may exhibit altered effector function compared to an anti-αvβ5 antibody population wherein Man₅GlcNAc₂N-glycan structure is not predominant Antibodies with predominantly this glycan structure exhibit decreased binding to FcγRIIa and FcγRIIb, increased binding to FcγRIIIa and FcγRIIIb, and increased binding to C1q subunit of the C1 complex (see US 2006-0257399). This glycan structure, when it is the predominant glycan structure, confers increased ADCC, increased CDC, increased serum half-life, increased antibody production of B cells, and decreased phagocytosis by macrophages.

In general, the glycosylation structures on a glycoprotein will vary depending upon the expression host and culturing conditions (Raju, T S. BioProcess International April 2003. 44-53). Such differences can lead to changes in both effector function and pharmacokinetics (Israel et al. Immunology, 1996; 89(4):573-578; Newkirk et al. P. Clin. Exp., 1996; 106(2):259-64). For example, galactosylation can vary with cell culture conditions, which may render some immunoglobulin compositions immunogenic depending on their specific galactose pattern (Patel et al., 1992. Biochem J. 285: 839-845). The oligosaccharide structures of glycoproteins produced by non-human mammalian cells tend to be more closely related to those of human glycoproteins. Further, protein expression host systems may be engineered or selected to express a predominant Ig glycoform or alternatively may naturally produce glycoproteins having predominant glycan structures. Examples of engineered protein expression host systems producing a glycoprotein having a predominant glycoform include gene knockouts/mutations (Shields et al., 2002, JBC, 277: 26733-26740); genetic engineering in (Umana et al., 1999, Nature Biotech., 17: 176-180) or a combination of both. Alternatively, certain cells naturally express a predominant glycoform—for example, chickens, humans and cows (Raju et al., 2000, Glycobiology, 10: 477-486). Thus, the expression of an anti-αvβ5 antibody or antibody composition having altered glycosylation (e.g., predominantly one specific glycan structure) can be obtained by one skilled in the art by selecting at least one of many expression host systems. Protein expression host systems that may be used to produce anti-αvβ5 antibodies of the present invention include animal, plant, insect, bacterial cells and the like. For example, US 2007-0065909, 2007-0020725, and 2005-0170464 describe producing aglycosylated immunoglobulin molecules in bacterial cells. As a further example, Wright and Morrison produced antibodies in a CHO cell line deficient in glycosylation (1994 J Exp Med 180: 1087-1096) and showed that antibodies produced in this cell line were incapable of complement-mediated cytolysis. Other examples of expression host systems found in the art for production of glycoproteins include: CHO cells: Raju WO 99/22764 and Presta WO 03/35835; hybridoma cells: Trebak et al., 1999, J. Immunol. Methods, 230: 59-70; insect cells: Hsu et al., 1997, JBC, 272:9062-970, and plant cells: Gerngross et al., WO 04/74499. To the extent that a given cell or extract has resulted in the glycosylation of a given motif, art recognized techniques for determining if the motif has been glycosylated are available, for example, using gel electrophoresis and/or mass spectroscopy.

Additional methods for altering glycosylation sites of antibodies are described, e.g., in U.S. Pat. No. 6,350,861 and U.S. Pat. No. 5,714,350, WO 05/18572 and WO 05/03175; these methods can be used to produce anti-αvβ5 antibodies of the present invention with altered, reduced, or no glycosylation.

The aglycosyl anti-αvβ5 antibodies with reduced effector function may be antibodies that comprise modifications or that may be conjugated to comprise a functional moiety. Such moieties include a blocking moiety (e.g., a PEG moiety, cysteine adducts, etc.), a detectable moiety (e.g., fluorescent moieties, radioisotopic moieties, radiopaque moieties, etc., including diagnostic moieties), a therapeutic moiety (e.g., cytotoxic agents, anti-inflammatory agents, immunomodulatory agents, anti-infective agents, anti-cancer agents, anti-neurodegenerative agents, radionuclides, etc.), and/or a binding moiety or bait (e.g., that allows the antibody to be pre-targeted to a tumor and then to bind a second molecule, composed of the complementary binding moiety or prey and a detectable moiety or therapeutic moiety, as described above).

### Indications

The anti-αvβ5 antibodies or antigen-binding fragments thereof described herein can be used to treat, prevent, or reduce the symptoms or severity of acute kidney injury in a subject (e.g. a human subject) in need thereof. These antibodies and antibody fragments are also useful in preventing the development of chronic kidney disease in a subject in need thereof. In certain embodiments, the antibodies and antibody fragments are useful in preventing the development of chronic kidney disease in a subject in need thereof following an insult that can cause or causes acute kidney injury. In addition, the antibodies or antigen-binding fragments thereof described herein can be used in methods for protecting a kidney from acute or chronic kidney injury in a subject in need thereof. Furthermore, the antibodies or antigen-binding fragments thereof described herein can be used in methods for treating patients with renal insufficiency or renal failure, attributable at least in part to use of a drug or chemical.

Acute kidney injury is commonly divided into two major categories based on the type of insult. The first category is ischemic acute kidney injury (alternatively referred to as kidney hypoperfusion) and the second category is nephrotoxic acute kidney injury. The former results from impaired blood flow (kidney hypoperfusion) and oxygen delivery to the kidney; whereas, the latter results from a toxic insult to the kidney. Both of these categories of insults can lead to a secondary condition called acute tubular necrosis (ATN).

The most common causes of ischemic acute kidney injury are intravascular volume depletion, reduced cardiac output, systemic vasodilatation, and renal vasoconstriction. Intravascular volume depletion can be caused by hemorrhage (e.g., following surgery, postpartum, or trauma); gastrointestinal loss (e.g., from diarrhea, vomiting, nasogastric loss); renal losses (e.g., caused by diuretics, osmotic diuresis, diabetes insipidus); skin and mucous membrane losses (e.g., burns, hyperthermia); nephrotic syndrome; cirrhosis; or capillary leak. Reduced cardiac output can be due to cardiogenic shock, pericardial disease (e.g. restrictive, constrictive, tamponade), congestive heart failure, valvular heart disease, pulmonary disease (e.g., pulmonary hypertension, pulmonary embolism), or sepsis. Systemic vasodilation can be the result of cirrhosis, anaphylaxis, or sepsis. Finally, renal vasoconstriction can be caused by early sepsis, hepatorenal syndrome, acute hypercalcemia, drug-related (e.g., norepinephrine, vasopressin, nonsteroidal anti-inflammatory drugs, angiotensin-converting enzyme inhibitors, calcineurin inhibitors), or use of a radiocontrast agent. The antibodies or antigen-binding fragments thereof described herein can be used to treat or reduce the symptoms or severity of acute kidney injury or any other kidney injury caused by any of the above mentioned causes of ischemic acute kidney injury. In addition, the antibodies or antigen-binding fragments thereof described herein can be used to prevent the development of acute kidney injury or any other kidney injury following exposure to the above mentioned causes of ischemic acute kidney injury.

Nephrotoxic acute kidney injury is often associated with exposure to a nephrotoxin such as a nephrotoxic drug. Examples of nephrotoxic drugs include an antibiotic (e.g., aminoglycosides such as gentamicin), a chemotherapeutic agent (e.g., cis-platinum), a calcineurin inhibitor (e.g., tacrolimus, cyclosporine), cephalosporins such as cephaloridine, cyclosporin, pesticides (e.g., paraquat), environmental contaminants (e.g., trichloroethylene, dichloroacetylene), amphotericin B, puromcyin, aminonucleoside (PAN), a radiographic contrast agent (e.g., acetrizoate, diatrizoate, iodamide, ioglicate, iothalamate, ioxithalamate, metrizoate, metrizamide, iohexol, iopamidol, iopentol, iopromide, and ioversol), a nonsteroidal anti-inflammatory, an anti-retroviral, an immunosuppressant, an oncological drug, or an ACE inhibitor. A nephrotoxin can be, for example, a trauma injury, a crush injury, an illicit drug, analgesic abuse, a gunshot wound, or a heavy metal. The antibodies or antigen-binding fragments thereof described herein can be used to treat or reduce the symptoms or severity of acute kidney injury or any other kidney injury caused by any of the above mentioned causes of nephrotoxic acute kidney injury. In addition, the antibodies or antigen-binding fragments thereof described herein can be used to prevent the development of acute kidney injury or any other kidney injury following exposure to the above mentioned causes of nephrotoxic acute kidney injury.

In certain embodiments, the antibodies or antigen-binding fragments thereof described herein can be used to prevent the development of ATN following exposure to an insult such as ischemia or nephrotoxins/nephrotoxic drugs. In certain embodiments, the antibodies or antigen-binding fragments thereof described herein can be used to treat or reduce the symptoms or severity of ATN following ischemia or exposure to nephrotoxins/nephrotoxic drugs.

In certain embodiments, the antibodies or antigen-binding fragments thereof described herein can be used to prevent a drop in glomerular filtration following ischemia or exposure to nephrotoxins/nephrotoxic drugs. In some embodiments, the antibodies or antigen-binding fragments thereof described herein can be used to prevent tubular epithelial injury and/or necrosis following ischemia or exposure to nephrotoxins/nephrotoxic drugs. In some embodiments, the antibodies or antigen-binding fragments thereof described herein can be used to decrease the microvascular permeability, improve vascular tone, and/or reduce inflammation of endothelial cells. In other embodiments, the antibodies or antigen-binding fragments thereof described herein can be used to restore blood flow in the kidney following ischemia or exposure to nephrotoxins/nephrotoxic drugs. In further embodiments, the antibodies or antigen-binding fragments thereof described herein can be used to prevent chronic renal failure.

The antibodies or antigen-binding fragments thereof described herein can also be used to treat or prevent acute kidney injury resulting from surgery complicated by hypoperfusion. In certain specific embodiments, the surgery is one of cardiac surgery, major vascular surgery, major trauma, or surgery associated with treating a gunshot wound. In one embodiment, the cardiac surgery is coronary artery bypass grafting (CABG). In another embodiment, the cardiac surgery is valve surgery.

In some embodiments, the antibodies or antigen-binding fragments thereof described herein can be used to treat or prevent acute kidney injury following organ transplantation such as kidney transplantation or heart transplantation.

In some embodiments, the antibodies or antigen-binding fragments thereof described herein can be used to treat or prevent acute kidney injury following reduced effective arterial volume and kidney hypoperfusion.

In some embodiments, the antibodies or antigen-binding fragments thereof described herein can be used to treat or prevent acute kidney injury in a subject who is taking medication (e.g., an anticholinergic) that interferes with normal emptying of the bladder. In certain embodiments, the antibodies or antigen-binding fragments thereof described herein can be used to treat or prevent acute kidney injury in a subject who has an obstructed urinary catheter. In some embodiments, the antibodies or antigen-binding fragments thereof described herein can be used to treat or prevent acute kidney injury in a subject who is taking a drug that causes crystalluria. In some embodiments, the antibodies or antigen-binding fragments thereof described herein can be used to treat or prevent acute kidney injury in a subject who is taking a drug that causes or leads to myoglobinuria. In some embodiments, the antibodies or antigen-binding fragments thereof described herein can be used to treat or prevent acute kidney injury in a subject who is taking a drug that causes or leads to cystitis.

In some embodiments, the antibodies or antigen-binding fragments thereof described herein can be used to treat or prevent acute kidney injury in a subject who has benign prostatic hypertrophy or prostate cancer.

In some embodiments, the antibodies or antigen-binding fragments thereof described herein can be used to treat or prevent acute kidney injury in a subject who has a kidney stone.

In some embodiments, the antibodies or antigen-binding fragments thereof described herein can be used to treat or prevent acute kidney injury in a subject who has an abdominal malignancy (e.g., ovarian cancer, colorectal cancer).

In certain embodiments, the antibodies or antigen-binding fragments thereof described herein can be used to treat or prevent acute kidney injury, wherein sepsis does not cause or result in the acute kidney injury.

Acute kidney injury typically occurs within hours to days following the original insult (e.g., ischemia or nephrotoxin insult). Thus, the antibodies or antigen-binding fragments thereof described herein can be administered before the insult, or within an hour to 30 days (e.g., 0.5 hours, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 15 days, 20 days, 25 days, 28 days, or 30 days) after the insult (e.g., a surgery or nephrotoxin insult described herein).

A subject can be determined to have, or have the risk of developing, acute kidney injury based on, e.g., the Risk Injury Failure Loss ESRD (RIFLE) criteria or the Acute Kidney Injury Network criteria (Bagshaw et al., Nephrol. Dial. Transplant., 23 (5):1569-1574 (2008); Lopes et al., Clin. Kidney J., 6(1):8-14 (2013)).

In certain embodiments, the methods of this disclosure involve determining measuring the levels of one or more of: serum, plasma or urine creatinine or blood urea nitrogen (BUN); measuring the levels of serum or urine neutrophil gelatinase-associated lipocalin (NGAL), serum or urine interleukin-18 (IL-18), serum or urine cystatin C, or urine KIM-1, compared to a healthy control subject, to assess whether the subject has, or has a risk of developing, acute kidney injury.

The efficacy of the antibodies of the invention can be assessed in various animal models. Animal models for acute kidney injury include those disclosed in e.g., Heyman et al., Contrin. Nephrol., 169:286-296 (2011); Heyman et al., Exp. Opin. Drug Disc., 4(6): 629-641 (2009); Morishita et al., Ren. Fail., 33(10):1013-1018 (2011); Wei Q et al., Am. J. Physiol. Renal Physiol., 303(11):F1487-94 (2012).

The efficacy of treatments may be measured by a number of available diagnostic tools, including physical examination, blood tests, measurements of blood systemic and capillary pressure, proteinuria (e.g., albuminuria), microscopic and macroscopic hematuria, assessing serum creatinine levels, assessment of the glomerular filtration rate, histological evaluation of renal biopsy, urinary albumin creatinine ratio, albumin excretion rate, creatinine clearance rate, 24-hour urinary protein secretion, and renal imaging (e.g., MRI, ultrasound).

### Pharmaceutical Compositions

An anti-αvβ5 antibody or antigen-binding fragment thereof described herein can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

In one embodiment, an anti-αvβ5 antibody described herein is formulated with excipient materials, such as sodium citrate, sodium dibasic phosphate heptahydrate, sodium monobasic phosphate, Tween-80, and a stabilizer. It can be provided, for example, in a buffered solution at a suitable concentration and can be stored at 2-8° C. In some other embodiments, the pH of the composition is between about 5.5 and 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5).

The pharmaceutical compositions can also include agents that reduce aggregation of the αvβ5 antibody or antigen-binding fragment thereof when formulated. Examples of aggregation reducing agents include one or more amino acids selected from the group consisting of methionine, arginine, lysine, aspartic acid, glycine, and glutamic acid. These amino acids may be added to the formulation to a concentration of about 0.5 mM to about 145 mM (e.g., 0.5 mM, 1 mM, 2 mM, 5 mM, 10 mM, 25 mM, 50 mM, 100 mM). The pharmaceutical compositions can also include a sugar (e.g., sucrose, trehalose, mannitol, sorbitol, or xylitol) and/or a tonicity modifier (e.g., sodium chloride, mannitol, or sorbitol) and/or a surfactant (e.g., polysorbate-20 or polysorbate-80).

Such compositions can be administered by a parenteral mode (e.g., intravenous, subcutaneous, intraperitoneal, or intramuscular injection. The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In one embodiment, the anti-αvβ5 antibody or antigen-binding fragment thereof compositions are administered subcutaneously. In one embodiment, the anti-αvβ5 or the antibody or antigen-binding fragment thereof compositions are administered intravenously. In one embodiment, the anti-αvβ5 or the antibody or antigen-binding fragment thereof compositions are administered intraarterially.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the anti-αvβ5 antibody or antigen-binding fragment thereof may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

In one embodiment, the pharmaceutical formulation comprises an anti-αvβ5 antibody or antigen-binding fragment thereof at a concentration of about 0.5 mg/mL to 500 mg/mL (e.g., 0.5 mg/mL, 1 mg/mL, 5 mg/mL, 10 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 250 mg/mL, 300 mg/mL, 350 mg/mL, 400 mg/mL, 450 mg/mL, 500 mg/mL), formulated with a pharmaceutically acceptable carrier. In some embodiments, the anti-αvβ5 antibody or antigen-binding fragment thereof is formulated in sterile distilled water or phosphate buffered saline. The pH of the pharmaceutical formulation may be between 5.5 and 7.5 (e.g., 5.5, 5.6, 5.7, 5.8, 5.9, 6.0, 6.1, 6.2 6.3, 6.4 6.5, 6.6 6.7, 6.8, 6.9 7.0, 7.1, 7.3, 7.4, 7.5).

### Administration

The anti-αvβ5 antibody or antigen-binding fragment thereof can be administered to a subject, e.g., a subject in need thereof, for example, a human subject, by a variety of methods. For many applications, the route of administration is one of: intravenous injection or infusion (IV), subcutaneous injection (SC), intraarterial, intraperitoneally (IP), or intramuscular injection. It is also possible to use inhalation delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody or antigen-binding fragment thereof can also be tailored for the individual case.

The antibody or antigen-binding fragment thereof can be administered as a fixed dose, or in a mg/kg dose. The dose can also be chosen to reduce or avoid production of antibodies against the anti-αvβ5 antibody. Dosage regimens are adjusted to provide the desired response, e.g., a therapeutic response or a combinatorial therapeutic effect. Generally, doses of the anti-αvβ5 antibody or antigen-binding fragment thereof (and optionally a second agent) can be used in order to provide a subject with the agent in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used. In certain embodiments, a subject in need of treatment with an anti-αvβ5 antibody or antigen-binding fragment thereof is administered the antibody at a dose of 1 mg/kg to 30 mg/kg. In some embodiments, a subject in need of treatment with an anti-αvβ5 antibody or antigen-binding fragment thereof is administered the antibody at a dose of 1 mg/kg, 2 mg/kg, 4 mg/kg, 5 mg/kg, 7 mg/kg 10 mg/kg, 12 mg/kg, 15 mg/kg, 20 mg/kg, 25 mg/kg, 28 mg/kg, 30 mg/kg, 35 mg/kg, 40 mg/kg, or 50 mg/kg. In a specific embodiment, the antibodies or antigen-binding fragments thereof are administered subcutaneously at a dose of 1 mg/kg to 3 mg/kg. In another embodiment, the antibodies or antigen-binding fragments thereof are administered intravenously at a dose of 4 mg/kg to 30 mg/kg.

A composition may comprise about 1 mg/mL to 100 mg/ml or about 10 mg/mL to 100 mg/ml or about 50 to 250 mg/mL or about 100 to 150 mg/ml or about 100 to 250 mg/ml of anti-αvβ5 antibody or an antigen-binding fragment thereof. In certain embodiments, the anti-αvβ5 antibody or antigen-binding fragment thereof in a composition is predominantly in monomeric form, e.g., at least about 90%, 92%, 94%, 96%, 98%, 98.5% or 99% in monomeric form. Certain anti-αvβ5 antibody or antigen-binding fragment thereof compositions may comprise less than about 5, 4, 3, 2, 1, 0.5, 0.3 or 0.1% aggregates, as detected, e.g., by UV at A280 nm. Certain anti-αvβ5 antibody or antigen-binding fragment thereof compositions comprise less than about 5, 4, 3, 2, 1, 0.5, 0.3, 0.2 or 0.1% fragments, as detected, e.g., by UV at A280 nm.

Dosage unit form or “fixed dose” as used herein refers to physically discrete units suited as unitary dosages for the subjects to be treated; each unit contains a predetermined quantity of anti-αvβ5 antibody calculated to produce the desired therapeutic effect in association with the required pharmaceutical carrier and optionally in association with the other agent. Single or multiple dosages may be given. Alternatively, or in addition, the antibody may be administered via continuous infusion.

An anti-αvβ5 antibody or antigen-binding fragment thereof dose can be administered, e.g., at a periodic interval over a period of time (a course of treatment) sufficient to encompass at least 2 doses, 3 doses, 5 doses, 10 doses, or more, e.g., once, twice, or thrice daily, or about one to six, seven, eight, nine, or ten times per week, or weekly, biweekly (every two weeks), every three weeks, or monthly. Preferably the dose(s) is/are administered within 0.5 hours, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 1 day, 2 days, 3 days, 5 days, 7 days, 12 days, 15 days, 20 days, 25 days or 30 days of the insult (e.g., ischemic or nephrotoxic) that leads to AKI. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

If a subject is at risk for developing a disorder described herein, the antibody or antigen-binding fragment thereof can be administered before the full onset of the disorder, e.g., as a preventative measure. The duration of such preventative treatment can be a single dosage of the antibody or antigen-binding fragment thereof or the treatment may continue (e.g., multiple dosages). For example, a subject at risk for the disorder or who has a predisposition for the disorder may be treated with the antibody or antigen-binding fragment thereof for hours, days, weeks, or a month so as to prevent the disorder from occurring or fulminating. For example, a subject who is expected to be exposed to an insult (e.g., ischemic or nephrotoxic) that can lead to acute kidney injury, can be administered an antibody or antigen-binding fragment thereof described herein a month, a week, 6 days, 5 days, 4 days, 3 days, 2 days, 1 day, 0.5 hours, 0.4 hours, 0.3 hours, 0.2 hours, 0.1 hours before, or substantially at the same time as, the insult.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

In certain embodiments, the anti-αvβ5 antibody or antigen-binding fragment thereof is administered subcutaneously at a concentration of about 1 mg/mL to about 500 mg/mL (e.g., 1 mg/mL, 2 mg/mL, 3 mg/mL, 4 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 400 mg/mL, 450 mg/mL). In one embodiment, the anti-αvβ5 antibody or antigen-binding fragment thereof is administered subcutaneously at a concentration of 50 mg/mL. In another embodiment, the anti-αvβ5 antibody or antigen-binding fragment thereof is administered intravenously at a concentration of about 1 mg/mL to about 500 mg/mL. In a particular embodiment, the anti-αvβ5 antibody or antigen-binding fragment thereof is administered intravenously at a concentration of 50 mg/mL.

The anti-αvβ5 antibody or antigen-binding fragment thereof can be administered to a patient in need thereof (e.g., a patient having, or at risk of developing, an acute kidney injury) in combination with a second therapeutic agent. For example, the second therapeutic agent can be an antagonist (e.g., antibodies, polypeptide antagonists, and/or small molecule antagonists) of one or more: other integrin receptors (e.g., αvβ5, αvβ6, α1β1, α4β1, αvβ8, αvβ1, etc.); cytokines (e.g., IL-1α, IL-6, IL-12); chemokines (e.g., CXCR4, MIP-1α); or a chemical moiety that is considered useful for treatment or prevention of acute kidney injury (e.g., AP214, THR-184, QPI-1002, an aromatic cationic peptide disclosed in US 2003/0017150). In certain embodiments, the second therapeutic agent is an anti-apoptosis/necrosis agent (e.g., a caspase inhibitor (e.g., nonselective caspase inhibitors, selective caspase 3 and 7 inhibitors, selective caspase 1 inhibitor), minocycline, guanosine, pifithrin-α, Poly ADP-Ribose Polymerase Inhibitor (PARP) inhibitor); an anti-inflammatory agent (e.g., Sphingosine 1 phosphate analog, Adenosine 2A agonist, α-MSH, IL-10, Fibrate, PPAR-γ agonist, Minocycline, Activated protein C, iNOS inhibitor); an anti-sepsis agent (e.g., insulin, activated protein C, ethyl pyruvate); a growth factor (e.g., recombinant erythropoietin, hepatocyte growth factor); a vasodilator (e.g., Carbon monoxide release compound and bilirubin, endothelin antagonist, fenoldopam, and ANP); a free radical scavenger (e.g., deferoxamine); or compounds such as neutrophil gelatinase-associated lipocalin, IL-6, C5a antagonists, IL-10, and α-melanocyte-stimulating hormone.

The anti-αvβ5 antibody or antigen-binding fragment thereof and the second therapeutic agent may be administered simultaneously or sequentially. In certain embodiments, the anti-αvβ5 antibody or antigen-binding fragment thereof and the second therapeutic agent can each be administered at either a subtherapeutic dose or a therapeutic dose.

### Devices and Kits for Therapy

Pharmaceutical compositions that include the anti-αvβ5 antibody or antigen-binding fragment thereof can be administered with a medical device. The device can be designed with features such as portability, room temperature storage, and ease of use so that it can be used in emergency situations, e.g., by an untrained subject or by emergency personnel in the field, removed from medical facilities and other medical equipment. The device can include, e.g., one or more housings for storing pharmaceutical preparations that include anti-αvβ5 antibody or antigen-binding fragment thereof, and can be configured to deliver one or more unit doses of the antibody. The device can be further configured to administer a second therapeutic agent, either as a single pharmaceutical composition that also includes the anti-αvβ5 antibody or antigen-binding fragment thereof or as two separate pharmaceutical compositions.

The pharmaceutical composition may be administered with a syringe. The pharmaceutical composition can also be administered with a needleless hypodermic injection device, such as the devices disclosed in U.S. Pat. No. 5,399,163; 5,383,851; 5,312,335; 5,064,413; 4,941,880; 4,790,824; or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other devices, implants, delivery systems, and modules are also known.

An anti-αvβ5 antibody or antigen-binding fragment thereof can be provided in a kit. In one embodiment, the kit includes (a) a container that contains a composition that includes anti-αvβ5 antibody, and optionally (b) informational material. The informational material can be descriptive, instructional, marketing or other material that relates to the methods described herein and/or the use of the agents for therapeutic benefit.

In an embodiment, the kit also includes a second therapeutic agent for treating or preventing acute kidney injury. For example, the kit includes a first container that contains a composition that includes the anti-αvβ5 antibody, and a second container that includes the second therapeutic agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the anti-αvβ5 antibody or antigen-binding fragment thereof, e.g., in a suitable dose, dosage form, or mode of administration (e.g., a dose, dosage form, or mode of administration described herein), to treat a subject who has had or who is at risk for an immunological disorder described herein. The information can be provided in a variety of formats, include printed text, computer readable material, video recording, or audio recording, or information that provides a link or address to substantive material, e.g., on the internet.

In addition to the antibody, the composition in the kit can include other ingredients, such as a solvent or buffer, a stabilizer, or a preservative. The antibody can be provided in any form, e.g., liquid, dried or lyophilized form, preferably substantially pure and/or sterile. When the agents are provided in a liquid solution, the liquid solution preferably is an aqueous solution. In certain embodiments, the antibody or antigen-binding fragment thereof in the liquid solution is at a concentration of about 25 mg/mL to about 250 mg/mL (e.g., 40 mg/mL, 50 mg/mL, 60 mg/mL, 75 mg/mL, 85 mg/mL, 100 mg/mL, 125 mg/mL, 150 mg/mL, 200 mg/mL). When the antibody or antigen-binding fragment is provided as a lyophilized product, the antibody or antigen-binding fragment is at about 75 mg/vial to about 200 mg/vial (e.g., 100 mg/vial, 108.5 mg/vial, 125 mg/vial, 150 mg/vial). The lyophilized powder is generally reconstituted by the addition of a suitable solvent. The solvent, e.g., sterile water or buffer (e.g., PBS), can optionally be provided in the kit. In certain embodiments, the lyophilized product is at 108.5 mg/vial and reconstituted to a liquid solution at a concentration of 75 mg/mL.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the anti-αvβ5 antibody or antigen-binding fragment thereof and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1

### Efficacy of an Anti-Integrin αvβ5 Antibody in the Rat Unilateral Ischemic Clamp Model

### Objective:

The dose response efficacy of ALULA in the rat kidney unilateral ischemic clamp model was determined after subcutaneous injection administration 18 hours pre- and 30 hours post-clamp release.

### Unilateral Clamp Ischemia Model Protocol

The animal was anesthetized with a Isofluorane/O₂ mixture, 5% for induction and 1-2% for maintenance of anesthesia. An induction chamber was used for induction and an anesthesia circuit was used during surgery. The abdomen was shaved with clippers and washed with germicidal soap and water, towel dried and swabbed with Betadine. The animal was placed on a sterile disposable absorbent towel over a warming pad thermostatically controlled by a rectal thermometer. The animal was monitored continuously for pulse, oximetry, respiratory rate, and blood pressure. The abdomen was opened using a 3 cm midline incision. Each kidney was isolated and the fat and connective tissue surrounding the renal artery and vein were dissected away using sterile cotton swabs. The right kidney was removed and the renal artery and vein sutured off.

Ischemia of the left kidney was initiated by clamping the renal artery and vein for 30 minutes using non-traumatic clamps on each renal pedicle. For sham surgery, the kidneys were isolated as above but not clamped. The incision was covered with sterile saline saturated gauze sponge during the ischemic period.

At the conclusion of the ischemic period, the clamps were removed and the kidneys were observed to insure rapid re-establishment of blood flow. The animal was rehydrated with 2 cc of sterile saline introduced into the abdominal cavity. The muscle layer was closed with 3-0 silk; the skin was closed with surgical 3-0 silk.

ALULA antibody or the control antibody (isotype negative mAB-1E6) was administered by subcutaneous injection, in an injection volume of 300 μl, 18 hours prior to clamping and then 30 hours after clamping. The following doses of ALULA were used for Study I: 10 mg/kg/body weight; 3 mg/kg/body weight; and 1 mg/kg/body weight. The following doses of ALULA were used for Study II: 10 mg/kg/body weight; 3 mg/kg/body weight; 1 mg/kg/body weight; 0.3 mg/kg/body weight; and 0.1 mg/kg/body weight.

Serum creatinine was measured at 0, 24, and 72 hours post surgery and the results were reported as mg per deciliter.

### Animals:

Species/Strain: Sprague Dawley Rats
Source: Harlan Laboratories
   P.O. Box 29176
   Indianapolis, Ind. 46229-0176
   USA
Age: 50-60 days
Body Weight Range: 250-320 g
Sex: Males
Group Size: 6 rats (Study I); 5 rats (Study II)
Total number: 24 rats (Study I); 25 rats (Study II)
Diet: Animals were provided an ad libitum commercial rodent diet, and free access to drinking water.
Environment: (i) Acclimatization of at least 5 days.
   (ii) All the animals were confined in a limited access facility with environmentally-controlled housing conditions throughout the entire study period, and maintained in accordance with approved standard operating procedures (SOPs).

### Blood Sampling

A 0.15 mL venous blood sample was drawn at study initiation for baseline creatinine measurement and at 0, 24, and 72 hours post surgery for pathological serum creatinine levels evaluation.

### Study Termination

72 hours post surgery, the study was terminated and the rats were euthanized by pentobarbital overdose followed by cervical dislocation.

### Tissue Collection

Following euthanasia, the left kidney of each individual rat was harvested and sliced into 2 longitudinal parts. One part was fixed in 10% buffered formalin and processed for paraffin embedding according to standard procedure for pathomorphological assessment. The second part of each kidney was immediately frozen in liquid nitrogen.

### Creatinine Measurement

Serum creatinine of all rats was measured at baseline and at days 1, 2, and 3 post-clamp. A 0.15 ml venous blood sample was drawn and centrifuged. The serum was removed and stored at +4° C. and saved for analysis. Creatinine concentration was measured on a Creatinine Analyzer 2 from Beckman Inc. The machine was standardized with a known control and the samples were run using a picric acid reaction.

### Pathomorphological Examination Method

Renal tissue samples are processed for paraffin embedding according to standard procedure and five micrometers sections are prepared and mounted on microscopic slides. Sections are stained with hematoxilin-eosin.

Slides are randomly numbered to blind the pathologist to the animal treatment.

Three pathomorphological features are assessed for the renal cortex and medulla separately: tubular necrosis, tubular dilatation and presence of tubular “casts” (necrotic debris within the tubular lumen). Grading of pathological changes is performed according to an established scoring system (K. J. Kelly et al., J. Clin. Invest., 108:1291-1298 (2001); Wei Q. et al., Am J Nephrol., 25(5):491-9 (2005)):

Grade 0=no pathological changes

Grade 1=feature involves 1-10% of the area

Grade 2=feature involves 10-25% of the area

Grade 3=feature involves 25-75% of the area

Grade 4=feature involves more than 75% of the area

### Results

The creatinine measurements (in mg/dL) at baseline, 24 hours, 48 hours, and 72 hours post-surgery from Study I are summarized in the Tables below. A value of 2.5-3.0 at 24 hours in untreated ischemic rats is common. This model has a rapid recovery phase, making it difficult to achieve statistical significance at times after 48-72 hours.

Group 1: Antibody: Isotype Negative mAb-1E6; Dose: 10 mg/kg/BW

| Rat | BW g | Baseline | BW g | 24 h | BW g | 48 h | BW g | 72 h |
|---|---|---|---|---|---|---|---|---|
| 1 | 310 | 0.5 | 291 | 2.8 | 284 | 1.6 | 279 | 1.6 |
| 2 | 309 | 0.3 | 288 | 3.1 | 272 | 1.9 | 275 | 1.3 |
| 3 | 316 | 0.5 | 307 | 3.9 | 289 | 4.6 | 275 | 4.1 |
| 4 | 309 | 0.5 | 301 | 1.8 | 292 | 1.4 | 282 | 0.9 |
| 5 | 315 | 0.5 | 310 | 3.8 | 289 | 3.0 | 285 | 2.9 |
| 6 | 321 | 0.4 | 311 | 3.4 | 298 | 2.9 | 301 | 2.4 |
| mean | 313 | 0.4 | 301 | 3.1 | 287 | 2.5 | 282 | 2.2 |

Group 2: Antibody: ALULA; Dose: 10 mg/kg/BW

| Rat | BW g | Baseline | BW g | 24 h | BW g | 48 h | BW g | 72 h |
|---|---|---|---|---|---|---|---|---|
| 1 | 310 | 0.4 | 293 | 2.3 | 279 | 1.7 | 280 | 1.3 |
| 2 | 315 | 0.5 | 303 | 1.6 | 291 | 1.2 | 296 | 0.8 |
| 3 | 313 | 0.3 | 285 | 1.8 | 282 | 1.6 | 278 | 1.1 |
| 4 | 318 | 0.4 | 304 | 0.8 | 304 | 0.7 | 304 | 0.8 |
| 5 | 312 | 0.4 | 309 | 1.0 | 304 | 0.8 | 308 | 0.7 |
| 6 | 315 | 0.5 | 295 | 0.7 | 293 | 0.8 | 293 | 0.7 |
| mean | 313 | 0.4 | 298 | 1.3 | 292 | 1.1 | 293 | 0.9 |

Group3: Antibody: ALULA; Dose: 3 mg/kg/BW

| Rat | BW g | Baseline | BW g | 24 h | BW g | 48 h | BW g | 72 h |
|---|---|---|---|---|---|---|---|---|
| 1 | 315 | 0.5 | 291 | 1.3 | 291 | 0.7 | 289 | 0.7 |
| 2 | 320 | 0.5 | 286 | 1.7 | 287 | 1.1 | 287 | 0.9 |
| 3 | 307 | 0.4 | 278 | 1.4 | 280 | 0.6 | 281 | 0.8 |
| 4 | 318 | 0.3 | 299 | 1.3 | 288 | 0.9 | 290 | 0.5 |
| 5 | 316 | 0.5 | 292 | 1.3 | 287 | 0.8 | 293 | 0.8 |
| 6 | 320 | 0.2 | 316 | 1.0 | 310 | 1.1 | 313 | 0.8 |
| mean | 316 | 0.4 | 293 | 1.3 | 290 | 0.8 | 292 | 0.7 |

Group 4: Antibody: ALULA; Dose: 1 mg/kg/BW

| Rat | BW g | Baseline | BW g | 24 h | BW g | 48 h | BW g | 72 h |
|---|---|---|---|---|---|---|---|---|
| 1 | 311 | 0.3 | 287 | 1.4 | 290 | 1.0 | 294 | 0.7 |
| 2 | 314 | 0.4 | 296 | 1.2 | 293 | 0.9 | 295 | 0.7 |
| 3 | 306 | 0.5 | 283 | 1.5 | 277 | 1.0 | 277 | 0.9 |
| 4 | 316 | 0.5 | 285 | 1.5 | 278 | 1.1 | 282 | 1.0 |
| 5 | 313 | 0.3 | 285 | 1.1 | 286 | 0.8 | 288 | 0.6 |
| 6 | 314 | 0.5 | 290 | 1.3 | 293 | 1.0 | 293 | 0.9 |
| mean | 312 | 0.4 | 287 | 1.3 | 286 | 0.9 | 288 | 0.8 |

The creatinine measurements (in mg/dL) at baseline, 24 hours, 48 hours, and 72 hours post-surgery from Study II are summarized in the Tables below.

Group 1: Antibody: Isotype Negative mAb-1E6; Dose: 3 mg/kg/BW

| Rat | BW g | Baseline | BW g | 24 h | BW g | 48 h | BW g | 72 h |
|---|---|---|---|---|---|---|---|---|
| 1 | 329 | 0.5 | 310 | 2.5 | 301 | 1.5 | 297 | 1.1 |
| 2 | 339 | 0.5 | 328 | 3.0 | 317 | 1.7 | 312 | 1.0 |
| 3 | 339 | 0.5 | 326 | 2.1 | 319 | 1.5 | 309 | 1.4 |
| 4 | 334 | 0.3 | 321 | 3.3 | 308 | 1.9 | 302 | 1.5 |
| 5 | 341 | 0.3 | 340 | 2.5 | 333 | 1.5 | 331 | 1.3 |
| mean | 336 | 0.4 | 325 | 2.6 | 315 | 1.6 | 310 | 1.2 |

Group 2: Antibody: ALULA; Dose: 3 mg/kg/BW

| Rat | BW g | Baseline | BW g | 24 h | BW g | 48 h | BW g | 72 h |
|---|---|---|---|---|---|---|---|---|
| 1 | 316 | 0.4 | 296 | 0.9 | 289 | 0.9 | 286 | 0.9 |
| 2 | 331 | 0.4 | 311 | 1.3 | 307 | 1.0 | 303 | 0.6 |
| 3 | 336 | 0.4 | 316 | 1.3 | 309 | 1.3 | 305 | 0.7 |
| 4 | 340 | 0.3 | 338 | 0.9 | 333 | 0.6 | 329 | 0.4 |
| 5 | 342 | 0.3 | 336 | 1.0 | 335 | 0.9 | 335 | 0.8 |
| mean | 333 | 0.3 | 319 | 1.0 | 314 | 0.9 | 311 | 0.6 |

Group 3: Antibody: ALULA; Dose: 1 mg/kg/BW

| Rat | BW g | Baseline | BW g | 24 h | BW g | 48 h | BW g | 72 h |
|---|---|---|---|---|---|---|---|---|
| 1 | 322 | 0.4 | 295 | 0.8 | 293 | 0.7 | 293 | 0.5 |
| 2 | 330 | 0.4 | 310 | 1.6 | 302 | 0.8 | 311 | 1.0 |
| 3 | 337 | 0.4 | 317 | 1.6 | 309 | 0.6 | 307 | 0.6 |
| 4 | 340 | 0.4 | 313 | 0.8 | 306 | 0.5 | 305 | 0.4 |
| 5 | 336 | 0.4 | 328 | 2.1 | 323 | 1.8 | 321 | 1.4 |
| mean | 333 | 0.4 | 312 | 1.3 | 306 | 0.8 | 307 | 0.7 |

Group 4: Antibody: ALULA; Dose: 0.3 mg/kg/BW

| Rat | BW g | Baseline | BW g | 24 h | BW g | 48 h | BW g | 72 h |
|---|---|---|---|---|---|---|---|---|
| 1 | 331 | 0.3 | 319 | 2.7 | 308 | 2.3 | 301 | 1.4 |
| 2 | 337 | 0.4 | 316 | 1.2 | 311 | 1.1 | 311 | 0.5 |
| 3 | 334 | 0.5 | 315 | 1.4 | 305 | 0.8 | 301 | 0.8 |
| 4 | 337 | 0.5 | 309 | 0.9 | 309 | 0.8 | 308 | 0.7 |
| 5 | 338 | 0.3 | 328 | 0.9 | 316 | 0.7 | 315 | 0.6 |
| mean | 335 | 0.4 | 317 | 1.4 | 309 | 1.1 | 307 | 0.8 |

Group 5: Antibody: ALULA; Dose: 0.1 mg/kg/BW

| Rat | BW g | Baseline | BW g | 24 h | BW g | 48 h | BW g | 72 h |
|---|---|---|---|---|---|---|---|---|
| 1 | 339 | 0.5 | 317 | 2.9 | 306 | 1.8 | 302 | 1.4 |
| 2 | 340 | 0.4 | 321 | 1.3 | 318 | 0.7 | 316 | 0.5 |
| 3 | 335 | 0.4 | 325 | 1.2 | 320 | 0.9 | 318 | 0.8 |
| 4 | 338 | 0.4 | 322 | 2.4 | 308 | 1.5 | 306 | 0.7 |
| 5 | 337 | 0.4 | 318 | 1.3 | 313 | 0.8 | 307 | 0.7 |
| mean | 337 | 0.4 | 320 | 1.8 | 313 | 1.1 | 309 | 0.8 |

The data from both Study I and Study II show that ALULA reduces creatinine levels even at the lowest doses tested compared to the control antibody.

### Example 2

### Efficacy of a Single Administration of an Anti-Integrin αvβ5 Antibody Pre-Injury in the Rat Unilateral Ischemic Clamp Model

### Objective

To establish the dose response efficacy of the monoclonal anti-αvβ5 antibody, ALULA, in the rat kidney unilateral ischemic clamp model after one subcutaneous injection at 18, 12, or 6 hours prior to clamping.

### Methods

### Unilateral Clamp Ischemia Model Protocol

The animal was anesthetized with an Isofluorane/O₂ mixture, 5% for induction and 1-2% for maintenance of anesthesia. An induction chamber was used for induction and an anesthesia circuit was used during surgery. The abdomen was shaved with clippers and washed with germicidal soap and water, towel dried and swabbed with Betadine. The animal was placed on a sterile disposable absorbent towel over a warming pad thermostatically controlled by a rectal thermometer. The animal was monitored continuously for pulse, oximetry, respiratory rate, and blood pressure. The abdomen was opened using a 3 cm midline incision. Each kidney was isolated and the fat and connective tissue surrounding the renal artery and vein were dissected away using sterile cotton swabs. The right kidney was removed and the renal artery and vein sutured off.

ALULA antibody or the control antibody (isotype negative mAB-1E6) was administered by subcutaneous injection, in an injection volume of 300 μl, 18, 12, or 6 hours prior to clamping. In this study ALULA and the control antibody were used at a dose of 3 mg/kg/body weight.

Ischemia of the left kidney was initiated by clamping the renal artery and vein for 30 minutes using non-traumatic clamps on each renal pedicle. For sham surgery, the kidneys were isolated as above but not clamped. The incision was covered with sterile saline saturated gauze sponge during the ischemic period.

At the conclusion of the ischemic period, the clamps were removed and the kidneys were observed to insure rapid re-establishment of blood flow. The animal was rehydrated with 2 cc of sterile saline introduced into the abdominal cavity. The muscle layer was closed with 3-0 silk; the skin was closed with surgical 3-0 silk.

Serum creatinine was measured at 0, 24, 48, and 72 hours post-surgery and the results were reported as mg per deciliter.

### Animals:

Species/Strain: Sprague Dawley Rats
Source: Harlan Laboratories
   P.O. Box 29176
   Indianapolis, Ind. 46229-0176
   USA
Age: 50-60 days
Body Weight Range: 250-320 g
Sex: Males
Group Size: 6 rats
Total number: 24 rats
Diet: Animals were provided an ad libitum commercial rodent diet and free access to drinking water.
Environment: (i) Acclimatization of at least 5 days.
   (ii) All the animals were confined in a limited access facility with environmentally-controlled housing conditions throughout the entire study period, and maintained in accordance with approved standard operating procedures (SOPs).

### Blood Sampling

A 0.15 mL venous blood sample was drawn at study initiation for baseline creatinine measurement and at 0, 24, 48, and 72 hours post-surgery for pathological serum creatinine levels evaluation.

### Study Termination

72 hours post-surgery, the study was terminated and the rats were euthanized by pentobarbital overdose followed by cervical dislocation.

### Creatinine Measurement

Serum creatinine of all rats was measured at baseline and at days 1, 2, and 3 post-clamp. A 0.15 ml venous blood sample was drawn and centrifuged. The serum was removed and stored at +4° C. and saved for analysis. Creatinine concentration was measured on a Creatinine Analyzer 2 from Beckman Inc. The machine was standardized with a known control and the samples were run using a picric acid reaction.

### Results

The creatinine measurements (in mg/dL) at baseline, 24 hours, 48 hours, and 72 hours post-surgery from this study are summarized in the Tables below. A value of 2.5-3.0 at 24 hours in untreated ischemic rats is common. This model has a rapid recovery phase, making it difficult to achieve statistical significance at times after 48-72 hours.

Group 1: Antibody: mAb-1E6; Dose: 3 mg/kg/BW; Administration: 18 hr Pre-Clamp

| Rat | BW g | Baseline | BW g | 24 h | BW g | 48 h | BW g | 72 h |
|---|---|---|---|---|---|---|---|---|
| 1 | 272 | 0.5 | 251 | 2.7 | 249 | 2.3 | 252 | 1.8 |
| 2 | 274 | 0.4 | 259 | 3.4 | 250 | 4.9 | died | died |
| 3 | 278 | 0.4 | 268 | 2.3 | 250 | 1.9 | 246 | 1.5 |
| 4 | 267 | 0.5 | 250 | 2.4 | 241 | 1.8 | 240 | 1.1 |
| 5 | 299 | 0.3 | 285 | 2.8 | 269 | 2.5 | 263 | 2.4 |
| 6 | 291 | 0.3 | 282 | 2.6 | 265 | 2.1 | 262 | 1.9 |
| mean | 280 | 0.4 | 265 | 2.7 | 254 | 2.5 | 252 | 1.7 |

Group 2: Antibody: ALULA; Dose: 3 mg/kg/BW; Administration: 18 hr Pre-Clamp

| Rat | BW g | Baseline | BW g | 24 h | BW g | 48 h | BW g | 72 h |
|---|---|---|---|---|---|---|---|---|
| 1 | 269 | 0.4 | 252 | 1.0 | 252 | 0.8 | 250 | 0.7 |
| 2 | 280 | 0.5 | 252 | 1.2 | 246 | 0.7 | 253 | 0.8 |
| 3 | 273 | 0.3 | 265 | 2.5 | 250 | 2.3 | 243 | 2.0 |
| 4 | 265 | 0.4 | 247 | 1.5 | 240 | 1.2 | 243 | 0.7 |
| 5 | 292 | 0.4 | 270 | 1.1 | 269 | 1.3 | 268 | 0.7 |
| 6 | 286 | 0.3 | 269 | 1.0 | 266 | 0.8 | 266 | 0.6 |
| mean | 277 | 0.3 | 259 | 1.3 | 253 | 1.1 | 253 | 0.9 |

Group 3: Antibody: ALULA; Dose: 3 mg/kg/BW; Administration: 12 hr Pre-Clamp

| Rat | BW g | Baseline | BW g | 24 h | BW g | 48 h | BW g | 72 h |
|---|---|---|---|---|---|---|---|---|
| 1 | 271 | 0.5 | 255 | 1.3 | 246 | 0.9 | 248 | 0.8 |
| 2 | 266 | 0.4 | 246 | 1.5 | 239 | 1.1 | 236 | 0.9 |
| 3 | 283 | 0.5 | 264 | 1.2 | 258 | 1.0 | 259 | 0.8 |
| 4 | 269 | 0.4 | 257 | 2.8 | 243 | 2.1 | 237 | 1.7 |
| 5 | 298 | 0.5 | 281 | 1.2 | 276 | 0.9 | 281 | 0.7 |
| 6 | 288 | 0.3 | 280 | 1.2 | 275 | 0.8 | 275 | 0.8 |
| mean | 279 | 0.4 | 263 | 1.5 | 256 | 1.1 | 256 | 0.9 |

Group 4: Antibody: ALULA; Dose: 3 mg/kg/BW; Administration: 6 hr Pre-Clamp

| Rat | BW g | Baseline | BW g | 24 h | BW g | 48 h | BW g | 72 h |
|---|---|---|---|---|---|---|---|---|
| 1 | 261 | 0.4 | 248 | 1.8 | 239 | 1.0 | 246 | 0.9 |
| 2 | 255 | 0.4 | 240 | 1.2 | 237 | 0.9 | 241 | 0.7 |
| 3 | 274 | 0.4 | 259 | 2.2 | 252 | 1.9 | 251 | 1.5 |
| 4 | 276 | 0.4 | 269 | 1.9 | 252 | 1.3 | 244 | 1.1 |
| 5 | 302 | 0.4 | 266 | 1.0 | 265 | 0.7 | 266 | 0.6 |
| 6 | 299 | 0.4 | 288 | 1.1 | 280 | 0.8 | 275 | 0.6 |
| mean | 277 | 0.4 | 261 | 1.5 | 254 | 1.1 | 253 | 0.9 |

These data show that ALULA reduces creatinine levels (compared to the control antibody) when only one dose of the antibody is administered prior to injury.

### Example 3

### Efficacy of a Single Administration of an Anti-Integrin αvβ5 Antibody Post-Injury in the Rat Unilateral Ischemic Clamp Model

### Objective

To establish the dose response efficacy of the monoclonal anti-αvβ5 antibody, ALULA, in the rat kidney unilateral ischemic clamp model after one subcutaneous injection at 12, 8, or 4 hours post-clamp release.

### Methods

### Unilateral Clamp Ischemia Model Protocol

The animal was anesthetized with an Isofluorane/O₂ mixture, 5% for induction and 1-2% for maintenance of anesthesia. An induction chamber was used for induction and an anesthesia circuit was used during surgery. The abdomen was shaved with clippers and washed with germicidal soap and water, towel dried and swabbed with Betadine. The animal was placed on a sterile disposable absorbent towel over a warming pad thermostatically controlled by a rectal thermometer. The animal was monitored continuously for pulse, oximetry, respiratory rate, and blood pressure. The abdomen was opened using a 3 cm midline incision. Each kidney was isolated and the fat and connective tissue surrounding the renal artery and vein were dissected away using sterile cotton swabs. The right kidney was removed and the renal artery and vein sutured off.

Ischemia of the left kidney was initiated by clamping the renal artery and vein for 30 minutes using non-traumatic clamps on each renal pedicle. For sham surgery, the kidneys were isolated as above but not clamped. The incision was covered with sterile saline saturated gauze sponge during the ischemic period.

At the conclusion of the ischemic period, the clamps were removed and the kidneys were observed to insure rapid re-establishment of blood flow. The animal was rehydrated with 2 cc of sterile saline introduced into the abdominal cavity. The muscle layer was closed with 3-0 silk; the skin was closed with surgical 3-0 silk.

ALULA antibody or the control antibody (isotype negative mAB-1E6) was administered by subcutaneous injection, in an injection volume of 300 μl, 12, 8, or 4 hours post-clamp release. In this study ALULA and the control antibody were used at a dose of 3 mg/kg/body weight.

Serum creatinine was measured at 0, 24, 48, and 72 hours post-surgery and the results were reported as mg per deciliter.

### Animals:

Species/Strain: Sprague Dawley Rats
Source: Harlan Laboratories
   P.O. Box 29176
   Indianapolis, Ind. 46229-0176
   USA
Age: 50-60 days
Body Weight Range: 250-320 g
Sex: Males
Group Size: 6 rats
Total number: 24 rats
Diet: Animals were provided an ad libitum commercial rodent diet and free access to drinking water.
Environment: (i) Acclimatization of at least 5 days.
   (ii) All the animals were confined in a limited access facility with environmentally-controlled housing conditions throughout the entire study period, and maintained in accordance with approved standard operating procedures (SOPs).

### Blood Sampling

A 0.15 mL venous blood sample was drawn at study initiation for baseline creatinine measurement and at 0, 24, 48, and 72 hours post-surgery for pathological serum creatinine levels evaluation.

### Study Termination

72 hours post-surgery, the study was terminated and the rats were euthanized by pentobarbital overdose followed by cervical dislocation.

### Creatinine Measurement

Serum creatinine of all rats was measured at baseline and at days 1, 2, and 3 post-clamp. A 0.15 ml venous blood sample was drawn and centrifuged. The serum was removed and stored at +4° C. and saved for analysis. Creatinine concentration was measured on a Creatinine Analyzer 2 from Beckman Inc. The machine was standardized with a known control and the samples were run using a picric acid reaction.

### Results

The creatinine measurements (in mg/dL) at baseline, 24 hours, 48 hours, and 72 hours post-surgery from this study are summarized in the Tables below. A value of 2.5-3.0 at 24 hours in untreated ischemic rats is common. This model has a rapid recovery phase, making it difficult to achieve statistical significance at times after 48-72 hours.

Group 1: Antibody: mAb-1E6; Dose: 3 mg/kg/BW; Administration: 12 hr Post-Clamp Release

| Rat | BW g | Baseline | BW g | 24 h | BW g | 48 h | BW g | 72 h |
|---|---|---|---|---|---|---|---|---|
| 1 | 283 | 0.3 | 265 | 3.9 | 255 | 3.9 | 251 | 3.2 |
| 2 | 305 | 0.5 | 287 | 3.4 | 270 | 3.4 | 270 | 2.0 |
| 3 | 297 | 0.5 | 274 | 2.4 | 264 | 2.2 | 256 | 1.8 |
| 4 | 314 | 0.5 | 301 | 3.7 | 284 | 3.3 | 278 | 2.1 |
| 5 | 324 | 0.4 | 306 | 3.6 | 301 | 3.4 | 295 | 2.3 |
| 6 | 310 | 0.5 | 286 | 2.1 | 276 | 1.9 | 276 | 1.3 |
| mean | 305 | 0.4 | 286 | 3.1 | 275 | 3.0 | 271 | 2.1 |

Group 2: Antibody: ALULA; Dose: 3 mg/kg/BW; Administration: 12 hr Post-Clamp Release

| Rat | BW g | Baseline | BW g | 24 h | BW g | 48 h | BW g | 72 h |
|---|---|---|---|---|---|---|---|---|
| 1 | 296 | 0.4 | 281 | 1.4 | 277 | 0.8 | 270 | 0.7 |
| 2 | 297 | 0.4 | 272 | 1.5 | 272 | 0.8 | 276 | 0.7 |
| 3 | 304 | 0.5 | 284 | 2.5 | 270 | 1.8 | 268 | 1.3 |
| 4 | 311 | 0.5 | 288 | 1.2 | 286 | 1.1 | 284 | 1.0 |
| 5 | 324 | 0.4 | 297 | 2.4 | 294 | 1.7 | 291 | 1.3 |
| 6 | 310 | 0.4 | 285 | 1.3 | 285 | 0.8 | 288 | 0.7 |
| mean | 307 | 0.4 | 284 | 1.7 | 280 | 1.1 | 279 | 0.9 |

Group 3: Antibody: ALULA; Dose: 3 mg/kg/BW; Administration: 8 hr Post-Clamp Release

| Rat | BW g | Baseline | BW g | 24 h | BW g | 48 h | BW g | 72 h |
|---|---|---|---|---|---|---|---|---|
| 1 | 307 | 0.5 | 292 | 3.7 | 282 | 3.4 | 268 | 2.4 |
| 2 | 284 | 0.4 | 261 | 1.9 | 262 | 1.7 | 266 | 1.3 |
| 3 | 304 | 0.5 | 289 | 3.1 | 281 | 1.9 | 278 | 1.1 |
| 4 | 304 | 0.5 | 297 | 1.8 | 291 | 1.8 | 293 | 0.9 |
| 5 | 334 | 0.4 | 319 | 3.3 | 314 | 2.1 | 308 | 1.7 |
| 6 | 326 | 0.5 | 294 | 1.5 | 299 | 1.1 | 293 | 0.8 |
| mean | 306 | 0.4 | 292 | 2.5 | 288 | 2.0 | 284 | 1.3 |

Group 4: Antibody: ALULA; Dose: 3 mg/kg/BW; Administration: 4 hr Post-Clamp Release

| Rat | BW g | Baseline | BW g | 24 h | BW g | 48 h | BW g | 72 h |
|---|---|---|---|---|---|---|---|---|
| 1 | 281 | 0.5 | 256 | 2.1 | 255 | 1.8 | 255 | 1.7 |
| 2 | 269 | 0.4 | 258 | 1.9 | 238 | 1.6 | 246 | 1.3 |
| 3 | 300 | 0.5 | 289 | 3.1 | 275 | 3.3 | 265 | 2.7 |
| 4 | 299 | 0.4 | 276 | 3.2 | 251 | 2.9 | 250 | 2.1 |
| 5 | 301 | 0.5 | 288 | 2.3 | 279 | 1.9 | 272 | 1.8 |
| 6 | 306 | 0.5 | 291 | 1.8 | 285 | 1.2 | 278 | 1.1 |
| mean | 292 | 0.4 | 276 | 2.4 | 263 | 2.1 | 261 | 1.7 |

These data show that ALULA surprisingly reduces creatinine levels (compared to the control antibody) when only one dose of the antibody is administered after injury.

### Example 4

### Efficacy of ALULA, in the Treatment of Renal Ischemia in the Rat Unilateral Ischemic Clamp Model with Pre-Clamp Treatment Time Course Analysis

### Objective:

To evaluate the efficacy of the monoclonal antibody ALULA in the rat kidney unilateral ischemic clamp model after subcutaneous (SQ) injection with one administration of ALULA or isotype control mAb at 6 hr pre-clamp and sacrificing animals at 24 hrs and 72 hours post-clamp.

### Methods:

The methods used in this study were substantially the same as those used in Example 3.

### Study Design:

| | | mg/kg | | | | |
|---|---|---|---|---|---|---|
| Group | Sample | BW | kg | mg/rat | N | Doses |
| 1 | Isotype negative mAb- | 3 | 0.3 | 0.9 | 6 | 1 |
| | IgG2b | | | | | |
| 2 | anti-avb5mAb, | 3 | 0.3 | 0.9 | 6 | 1 |
| | ALULA | | | | | |
| 3 | Isotype negative mAb- | 3 | 0.3 | 0.9 | 6 | 1 |
| | IgG2b | | | | | |
| 4 | anti-avb5mAb, ALULA | 3 | 0.3 | 0.9 | 6 | 1 |
| | | | | Dosed: | 24 | |
| | | | | Spare: | 0 | |
| | | | | Animals: | 24 | |

ALULA antibody or the control antibody was administered by subcutaneous injection, in an injection volume of 300 μl, 6 hours before clamping. A 0.15 mL venous blood sample was drawn at study initiation for baseline creatinine measurement and at 24, 48, and 72 hours post-surgery for pathological serum creatinine levels evaluation

### Results:

The creatinine measurements (in mg/dL) at baseline, 24 hours, 48 hours, and 72 hours post-surgery from this study are summarized in the Tables below. A value of 2.5-3.0 at 24 hours in untreated ischemic rats is common.

Group1: Antibody: Isotype Negative mAb-IgG2b; Dose: 3 mg/kg/BW

| Rat | BW g | baseline | BW g | 24 hr |
|---|---|---|---|---|
| 1 | 268 | 0.3 | 248 | 2.5 |
| 2 | 262 | 0.4 | 258 | 3.4 |
| 3 | 260 | 0.5 | 244 | 2.4 |
| 4 | 256 | 0.5 | 238 | 2.4 |
| 5 | 260 | 0.5 | 241 | 2.2 |
| 6 | 251 | 0.3 | 237 | 2.4 |
| mean | 259 | 0.4 | 244 | 2.5 |

Group2: Antibody: anti-αvβ5mAb, ALULA; Dose: 3 mg/kg/BW

| Rat | BW g | baseline | BW g | 24 hr |
|---|---|---|---|---|
| 1 | 255 | 0.3 | 242 | 1.4 |
| 2 | 263 | 0.5 | 240 | 1.2 |
| 3 | 255 | 0.5 | 238 | 1.1 |
| 4 | 266 | 0.4 | 251 | 1.2 |
| 5 | 262 | 0.4 | 248 | 1.7 |
| 6 | 260 | 0.4 | 239 | 1.1 |
| mean | 260 | 0.4 | 243 | 1.2 |

Group3: Antibody: Isotype Negative mAb-IgG2b; Dose: 3 mg/kg/BW

| Rat | BW g | baseline | BW g | 24 hr | BW g | 48 h | BW g | 72 h |
|---|---|---|---|---|---|---|---|---|
| 1 | 267 | 0.4 | 251 | 2.8 | 241 | 2.2 | 238 | 1.5 |
| 2 | 258 | 0.3 | 240 | 2.6 | 239 | 1.7 | 239 | 1.1 |
| 3 | 261 | 0.5 | 241 | 3.3 | 238 | 2.6 | 233 | 1.3 |
| 4 | 257 | 0.4 | 243 | 3.9 | 234 | 3.2 | 233 | 2.7 |
| 5 | 256 | 0.5 | 239 | 2.5 | 243 | 1.8 | 241 | 1.2 |
| 6 | 248 | 0.5 | 237 | 2.2 | 235 | 1.6 | 234 | 1.1 |
| mean | 257 | 0.4 | 241 | 2.8 | 238 | 2.1 | 236 | 1.4 |

Group 4: Antibody: anti-αvβ5mAb, ALULA; Dose: 3 mg/kg/BW

| Rat | BW g | baseline | BW g | 24 hr | BW g | 48 h | BW g | 72 h |
|---|---|---|---|---|---|---|---|---|
| 1 | 243 | 0.3 | 241 | 1.3 | 239 | 1.0 | 236 | 0.7 |
| 2 | 252 | 0.4 | 246 | 1.5 | 241 | 0.9 | 239 | 0.6 |
| 3 | 262 | 0.4 | 242 | 1.4 | 236 | 0.9 | 232 | 0.7 |
| 4 | 252 | 0.4 | 237 | 1.9 | 235 | 1.3 | 233 | 1.0 |
| 5 | 252 | 0.5 | 241 | 1.5 | 240 | 0.8 | 238 | 0.6 |
| 6 | 263 | 0.4 | 260 | 1.2 | 258 | 0.9 | 257 | 0.5 |
| mean | 254 | 0.4 | 244 | 1.4 | 241 | 0.9 | 239 | 0.6 |

These data show that ALULA reduces creatinine levels (compared to the control antibody) as early as 24 hours after injury when only one dose of the antibody is administered 6 hours prior to injury.

### Example 5

### Efficacy of Different Fc versions of Humanized ALULA in the Prevention of Renal Ischemia in the Rat Unilateral Ischemic Model with Pre-clamp Treatment

### Objective:

This study tested the efficacy of a humanized ALULA (containing all six CDRs of murine ALULA) expressed as a chimeric mAb with two different murine Fc tails (IgG2a or agly IgG1) compared to the murine ALULA mAb (murine IgG2b) or an isotype control mAb in the rat kidney unilateral ischemic clamp model after subcutaneous (SQ) injection administration 6 hr pre clamp release. This study compared the activity of the humanized ALULA agly IgG1 mAb to the humanized ALULA antibody containing a murine IgG2a Fc tail (murine IgG2a Fc is expected to have full effector function in rat). The original murine ALULA was included as a control.

### Methods:

The methods used in this study were substantially the same as those used in Example 3.

### Study Design:

| | | mg/kg | | | | |
|---|---|---|---|---|---|---|
| Group | Sample | BW | kg | mg/rat | N | Doses |
| 1 | Isotype negative mAb- | 1 | 0.3 | 0.3 | 6 | 1 |
| | IgG2b | | | | | |
| 2 | anti-avb5mAb, | 1 | 0.3 | 0.3 | 6 | 1 |
| | ALULA IgG2b | | | | | |
| | (original mu mAb) | | | | | |
| 3 | anti-avb5mAb, | 1 | 0.3 | 0.3 | 6 | 1 |
| | ALULA agly IgG1 | | | | | |
| | (humanized/mu Fc) | | | | | |
| 4 | anti-avb5mAb, ALULA | 1 | 0.3 | 0.3 | 6 | 1 |
| | IgG2a (humanized/mu Fc) | | | | | |
| | | | | Dosed: | 24 | |
| | | | | Animals: | 24 | |

The ALULA antibodies or the control antibody were administered by subcutaneous injection, in an injection volume of 300 μl, 6 hours before clamping. A 0.15 mL venous blood sample was drawn at study initiation for baseline creatinine measurement and at 24, 48, and 72 hours post-surgery for pathological serum creatinine levels evaluation.

### Results:

The creatinine measurements (in mg/dL) at baseline, 24 hours, 48 hours, and 72 hours post-surgery from this study are summarized in the Tables below. A value of 2.5-3.0 at 24 hours in untreated ischemic rats is common.

Group1: Antibody: Isotype Negative mAb-IgG2b; Dose: 1 mg/kg/BW (6 hr Pre-Clamp).

| Rat | BW g | baseline | BW g | 24 hr | BW g | 48 h | BW g | 72 h |
|---|---|---|---|---|---|---|---|---|
| 1 | 284 | 0.4 | 261 | 2.7 | 252 | 1.3 | 250 | 0.5 |
| 2 | 272 | 0.4 | 254 | 3.0 | 244 | 1.1 | 239 | 0.8 |
| 3 | 279 | 0.4 | 260 | 4.0 | 243 | 2.1 | 234 | 1.6 |
| 4 | 273 | 0.5 | 265 | 3.5 | 251 | 2.3 | 239 | 1.9 |
| 5 | 288 | 0.4 | 274 | 2.5 | 269 | 1.1 | 264 | 0.8 |
| 6 | 277 | 0.5 | 270 | 4.8 | 261 | 2.4 | 250 | 1.8 |
| mean | 278 | 0.4 | 264 | 3.4 | 253 | 1.7 | 246 | 1.2 |

Group2: Antibody: ALULA IgG2b; Dose: 1 mg/kg/BW (6 hr Pre-Clamp).

| Rat | BW g | baseline | BW g | 24 hr | BW g | 48 h | BW g | 72 h |
|---|---|---|---|---|---|---|---|---|
| 1 | 277 | 0.4 | 260 | 2.3 | 246 | 2.0 | 246 | 1.6 |
| 2 | 276 | 0.5 | 264 | 1.5 | 254 | 1.0 | 245 | 0.6 |
| 3 | 290 | 0.5 | 271 | 1.7 | 260 | 0.8 | 259 | 0.8 |
| 4 | 271 | 0.4 | 257 | 1.9 | 247 | 1.5 | 243 | 1.0 |
| 5 | 287 | 0.4 | 264 | 1.2 | 264 | 0.5 | 263 | 0.5 |
| 6 | 276 | 0.4 | 264 | 1.5 | 254 | 1.3 | 252 | 0.9 |
| mean | 279 | 0.4 | 263 | 1.6 | 254 | 1.1 | 251 | 0.9 |

Group3: Antibody: Chimeric Humanized ALULA agly IgG1; Dose: 1 mg/kg/BW (6 hr Pre-Clamp).

| Rat | BW g | baseline | BW g | 24 hr | BW g | 48 h | BW g | 72 h |
|---|---|---|---|---|---|---|---|---|
| 1 | 281 | 0.5 | 255 | 1.2 | 248 | 0.6 | 251 | 0.6 |
| 2 | 278 | 0.4 | 265 | 1.3 | 257 | 0.8 | 258 | 0.6 |
| 3 | 277 | 0.4 | 248 | 1.1 | 239 | 0.5 | 245 | 0.6 |
| 4 | 287 | 0.5 | 261 | 1.0 | 255 | 0.6 | 257 | 0.5 |
| 5 | 274 | 0.4 | 263 | 2.0 | 245 | 1.5 | 238 | 1.2 |
| 6 | 291 | 0.5 | 273 | 2.4 | 269 | 1.7 | 268 | 1.7 |
| mean | 281 | 0.4 | 260 | 1.5 | 252 | 0.9 | 252 | 0.8 |

Group 4: Antibody: Chimeric Humanized ALULA IgG2a; Dose: 1 mg/Kg/BW (6 hr Pre-Clamp).

| Rat | BW g | baseline | BW g | 24 hr | BW g | 48 h | BW g | 72 h |
|---|---|---|---|---|---|---|---|---|
| 1 | 279 | 0.5 | 260 | 1.6 | 253 | 0.7 | 251 | 0.4 |
| 2 | 269 | 0.4 | 265 | 1.3 | 247 | 0.7 | 243 | 0.6 |
| 3 | 264 | 0.4 | 248 | 1.1 | 245 | 0.6 | 250 | 0.6 |
| 4 | 276 | 0.5 | 266 | 2.5 | 246 | 2.0 | 238 | 1.8 |
| 5 | 274 | 0.3 | 257 | 1.3 | 252 | 0.7 | 248 | 0.6 |
| 6 | 278 | 0.4 | 254 | 0.9 | 250 | 0.8 | 247 | 0.7 |
| mean | 273 | 0.4 | 258 | 1.4 | 248 | 0.9 | 246 | 0.7 |

The serum creatinine data above indicates that there is no difference in potency when comparing all three versions of the mAb ALULA. The fact that there is no loss of activity after eliminating effector function suggests that effector function is not required as part of the mechanism of action producing efficacy.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating, preventing, or reducing the severity of acute kidney injury in a human subject in need thereof, the method comprising administering to the human subject an effective amount of an antibody or an antigen-binding fragment thereof that specifically binds to the αvβ5 integrin.

2. The method of claim 1, wherein the antibody or the antigen-binding fragment thereof competes with an antibody produced by the hybridoma deposited as ATCC Deposit No. PTA-5817.

3. The method of claim 1, wherein the antibody or the antigen-binding fragment thereof comprises the heavy chain variable region CDR1, CDR2, and CDR3 according to the Kabat definition of the antibody produced by the hybridoma deposited as ATCC Deposit No. PTA-5817.

4. The method of claim 3, wherein the antibody or the antigen-binding fragment thereof further comprises the light chain variable region CDR1, CDR2, and CDR3 according to the Kabat definition of the antibody produced by the hybridoma deposited as ATCC Deposit No. PTA-5817.

5. The method of claim 1, wherein the antibody or the antigen-binding fragment thereof is a humanized form of the antibody produced by the hybridoma deposited as ATCC Deposit No. PTA-5817.

6. The method of claim 1, wherein the antibody or the antigen-binding fragment thereof is administered intravenously, subcutaneously, or intraarterially.

7. The method of claim 1, wherein the human subject has been identified as having acute kidney injury based on the Acute Kidney Injury Network criteria or Risk/Injury/Failure/Loss/ESRD criteria.

8. The method of claim 1, wherein the human subject has been identified as having an elevated level of serum creatinine, plasma creatinine, urine creatinine, or blood urea nitrogen, compared to a healthy control subject.

9. The method of claim 1, wherein the human subject has been identified as having an elevated level of serum or urine neutrophil gelatinase-associated lipocalin, serum or urine interleukin-18, serum or urine cystatin C, or urine KIM-1, compared to a healthy control subject.

10. The method of claim 1, wherein the acute kidney injury is an ischemic acute kidney injury.

11. The method of claim 10, wherein the human subject has been identified as having reduced effective arterial volume.

12. The method of claim 10, wherein the human subject has been identified as having intravascular volume depletion.

13. The method of claim 12, wherein the intravascular volume depletion is due to hemorrhage, gastrointestinal loss, renal loss, skin and mucous membrane loss, nephrotic syndrome, cirrhosis, or capillary leak.

14. The method of claim 10, wherein the human subject has been identified as having reduced cardiac output.

15. The method of claim 14, wherein the reduced cardiac output is due to cardiogenic shock, pericardial disease, congestive heart failure, valvular heart disease, pulmonary disease, or sepsis.

16. The method of claim 10, wherein the human subject has been identified as having systemic vasodilation.

17. The method of claim 16, wherein the systemic vasodilation is caused by cirrhosis, anaphylaxis, or sepsis.

18. The method of claim 10, wherein the human subject has been identified as having renal vasoconstriction.

19. The method of claim 18, wherein the renal vasoconstriction is caused by early sepsis, hepatorenal syndrome, acute hypercalcemia, a drug, or a radiocontrast agent.

20. The method of claim 1, wherein the acute kidney injury is a nephrotoxic acute kidney injury.

21. The method of claim 20, wherein the human subject has been exposed to a nephrotoxin.

22. The method of claim 21, wherein the nephrotoxin is a nephrotoxic drug selected from the group consisting of an antibiotic, a chemotherapeutic agent, a calcineurin inhibitor, amphotericin B, and a radiographic contrast agent.

23. The method of claim 21, wherein the nephrotoxin is an illicit drug or a heavy metal.

24. The method of claim 1, wherein the human subject has undergone a trauma injury or a crush injury.

25. The method of claim 1, wherein the human subject has undergone an organ transplant surgery.

26. The method of claim 25, wherein the organ transplant surgery is kidney transplant surgery or heart transplant surgery.

27. The method of claim 1, wherein the human subject has undergone a surgery complicated by hypoperfusion.

28. The method of claim 1, wherein the human subject has undergone cardiothoracic surgery or a vascular surgery.

29. The method of claim 1, wherein the human subject has taken medication that interferes with normal emptying of the bladder.

30. The method of claim 29, wherein the medication is an anticholinergic.

31. The method of claim 1, wherein the human subject has benign prostatic hypertrophy.

32. The method of claim 1, wherein the human subject has a cancer.

33. The method of claim 32, wherein the cancer is prostate cancer, ovarian cancer, or colorectal cancer.

34. The method of claim 1, wherein the human subject has a kidney stone.

35. The method of claim 1, wherein the human subject has an obstructed urinary catheter.

36. The method of claim 1, wherein the human subject has taken a drug that causes or leads to crystalluria, a drug that causes or leads to myoglobinuria, or a drug that causes or leads to cystitis.

37. The method of claim 1, wherein the human subject is administered a second therapeutic agent selected from the group consisting of an αvβ5 integrin inhibitor, an αvβ6 integrin inhibitor, a CXCR4 antagonist, an IL-6 inhibitor, an IL-1α inhibitor, an IL-12 inhibitor, a MIP-1-α inhibitor, AP214, THR-184, QPI-1002, a human alkaline phosphatase, an anti-apoptosis agent, an anti-necrosis agent, an anti-inflammatory agent, an anti-sepsis agent, a growth factor, a vasodilator, a free radical scavenger, neutrophil gelatinase-associated lipocalin, a C5a receptor antagonist, and α-melanocyte-stimulating hormone.


SEQUENCE LISTING (30 sequences)

SEQ ID NO: 1 - protein, 799 aa, Homo sapiens
MPRAPAPLYACLLGLCALLPRLAGLNICTSGSATSCEECLLIHPKCAWCSKEDFGSPRSITSRCDLRANLVKNGCGGEIESPASSFHVLRSLPLSSKGSGSAGWDVIQMTPQEIAVNLRPGDKTTFQLQVRQVEDYPVDLYYLMDLSLSMKDDLDNIRSLGTKLAEEMRKLTSNFRLGFGSFVDKDISPFSYTAPRYQTNPCIGYKLFPNCVPSFGFRHLLPLTDRVDSFNEEVRKQRVSRNRDAPEGGFDAVLQAAVCKEKIGWRKDALHLLVFTTDDVPHIALDGKLGGLVQPHDGQCHLNEANEYTASNQMDYPSLALLGEKLAENNINLIFAVTKNHYMLYKNFTALIPGTTVEILDGDSKNIIQLIINAYNSIRSKVELSVWDQPEDLNLFFTATCQDGVSYPGQRKCEGLKIGDTASFEVSLEARSCPSRHTEHVFALRPVGFRDSLEVGVTYNCTCGCSVGLEPNSARCNGSGTYVCGLCECSPGYLGTRCECQDGENQSVYQNLCREAEGKPLCSGRGDCSCNQCSCFESEFGKIYGPFCECDNFSCARNKGVLCSGHGECHCGECKCHAGYIGDNCNCSTDISTCRGRDGQICSERGHCLCGQCQCTEPGAFGEMCEKCPTCPDACSTKRDCVECLLLHSGKPDNQTCHSLCRDEVITWVDTIVKDDQEAVLCFYKTAKDCVMMFTYVELPSGKSNLTVLREPECGNTPNAMTILLAVVGSILLVGLALLAIWKLLVTIHDRREFAKFQSERSRARYEMASNPLYRKPISTHTVDFTFNKFNKSYNGTVD

SEQ ID NO: 2 - protein, 799 aa, Mus musculus
MPRVPATLYACLLGLCALVPRLAGLNICTSGSATSCEECLLIHPKCAWCSKEYFGNPRSITSRCDLKANLIRNGCEGEIESPASSTHVLRNLPLSSKGSSATGSDVIQMTPQEIAVSLRPGEQTTFQLQVRQVEDYPVDLYYLMDLSLSMKDDLENIRSLGTKLAEEMRKLTSNFRLGFGSFVDKDISPFSYTAPRYQTNPCIGYKLFPNCVPSFGFRHLLPLTDRVDSFNEEVRKQRVSRNRDAPEGGFDAVLQAAVCKEKIGWRKDALHLLVFTTDDVPHIALDGKLGGLVQPHDGQCHLNEANEYTASNQMDYPSLALLGEKLAENNINLIFAVTKNHYMLYKNFTALIPGTTVEILHGDSKNIIQLIINAYSSIRAKVELSVWDQPEDLNLFFTATCQDGISYPGQRKCEGLKIGDTASFEVSVEARSCPGRQAAQSFTLRPVGFRDSLQVEVAYNCTCGCSTGLEPNSARCSGNGTYTCGLCECDPGYLGTRCECQEGENQSGYQNLCREAEGKPLCSGRGECSCNQCSCFESEFGRIYGPFCECDSFSCARNKGVLCSGHGECHCGECKCHAGYIGDNCNCSTDVSTCRAKDGQICSDRGRCVCGQCQCTEPGAFGETCEKCPTCPDACSSKRDCVECLLLHQGKPDNQTCHHQCKDEVITWVDTIVKDDQEAVLCFYKTAKDCVMMFSYTELPNGRSNLTVLREPECGSAPNAMTILLAVVGSILLIGMALLAIWKLLVTIHDRREFAKFQSERSRARYEMASNPLYRKPISTHTVDFAFNKFNKSYNGSVD

SEQ ID NO: 3 - protein, 5 aa, Artificial Sequence
  source: /note=“Description of Artificial Sequence Synthetic peptide”
SYWMH

SEQ ID NO: 4 - protein, 17 aa, Artificial Sequence
  source: /note=“Description of Artificial Sequence Synthetic peptide”
AIYPGNSDTSYNQKFKG

SEQ ID NO: 5 - protein, 10 aa, Artificial Sequence
  source: /note=“Description of Artificial Sequence Synthetic peptide”
TTYGYDWFAY

SEQ ID NO: 6 - protein, 17 aa, Artificial Sequence
  source: /note=“Description of Artificial Sequence Synthetic peptide”
KSSQSVLYSSNQKNYLA

SEQ ID NO: 7 - protein, 7 aa, Artificial Sequence
  source: /note=“Description of Artificial Sequence Synthetic peptide”
WASTRES

SEQ ID NO: 8 - protein, 8 aa, Artificial Sequence
  source: /note=“Description of Artificial Sequence Synthetic peptide”
HQYLSSLT

SEQ ID NO: 9 - protein, 119 aa, Artificial Sequence
  source: /note=“Description of Artificial Sequence Synthetic polypeptide”
EVQVQQSGTVLARPGASVKMSCKASGYTFTSYWMHWVKQRPGQGLEWIGAIYPGNSDTSYNQKFKGKAKLTAVTSPNTAYMELSSLTNEDSAVYYCTTTTYGYDWFAYWGQGTLVTVSA

SEQ ID NO: 10 - DNA, 357 bp, Artificial Sequence
  source: /note=“Description of Artificial Sequence Synthetic polynucleotide”
gaggttcaggtccagcagtctgggactgtgctggcaaggcctggggcttcagtgaagatgtcctgcaaggcttctggctacacctttaccagctactggatgcactgggtaaaacagaggcctggacagggtctggaatggattggcgctatttatcctggaaatagtgatactagctacaaccagaagttcaagggcaaggccaaactgactgcagtcacatcccccaacactgcctacatggagctcagcagcctgacaaatgaggactctgcggtctattactgtacaaccactacatatggttacgactggtttgcttactggggccaagggactctggtcactgtctctgca

SEQ ID NO: 11 - protein, 112 aa, Artificial Sequence
  source: /note=“Description of Artificial Sequence Synthetic polypeptide”
NIMMTQSPSSLTVSAGEKVTMSCKSSQSVLYSSNQKNYLAWYQQKPGQSPKLLIYWASTRESGVPDRFTGSGSGTDFTLTISSVQAEDLAVYYCHQYLSSLTFGAGTKLELK

SEQ ID NO: 12 - DNA, 336 bp, Artificial Sequence
  source: /note=“Description of Artificial Sequence Synthetic polynucleotide”
aacattatgatgacacagtcgccatcatctctgactgtgtctgcaggagaaaaggtcactatgagctgtaagtccagtcaaagtgttttatacagttcaaatcagaagaactacttggcctggtaccagcagaaaccagggcagtctcctaaactgctgatctactgggcatccactagggaatctggtgtccctgatcgcttcacaggcagtggatctgggacagattttactcttaccatcagcagtgtacaagctgaagacctggcagtttattactgtcatcaatacctctcctcgctcacgttcggtgctgggaccaagctggagctgaaa

SEQ ID NO: 13 - protein, 4 aa, Artificial Sequence
  source: /note=“Description of Artificial Sequence Synthetic peptide”
GGGS

SEQ ID NO: 14 - protein, 4 aa, Artificial Sequence
  source: /note=“Description of Artificial Sequence Synthetic peptide”
SGGG

SEQ ID NO: 15 - protein, 5 aa, Artificial Sequence
  source: /note=“Description of Artificial Sequence Synthetic peptide”
GGGGS

SEQ ID NO: 16 - protein, 5 aa, Artificial Sequence
  source: /note=“Description of Artificial Sequence Synthetic peptide”
SGGGG

SEQ ID NO: 17 - protein, 6 aa, Artificial Sequence
  source: /note=“Description of Artificial Sequence Synthetic peptide”
GGGGGS

SEQ ID NO: 18 - protein, 6 aa, Artificial Sequence
  source: /note=“Description of Artificial Sequence Synthetic peptide”
SGGGGG

SEQ ID NO: 19 - protein, 7 aa, Artificial Sequence
  source: /note=“Description of Artificial Sequence Synthetic peptide”
GGGGGGS

SEQ ID NO: 20 - protein, 7 aa, Artificial Sequence
  source: /note=“Description of Artificial Sequence Synthetic peptide”
SGGGGGG

SEQ ID NO: 21 - protein, 7 aa, Artificial Sequence
  source: /note=“Description of Artificial Sequence Synthetic peptide”
GYTFTSY

SEQ ID NO: 22 - protein, 10 aa, Artificial Sequence
  source: /note=“Description of Artificial Sequence Synthetic peptide”
GYTFTSYWMH

SEQ ID NO: 23 - protein, 6 aa, Artificial Sequence
  source: /note=“Description of Artificial Sequence Synthetic peptide”
TSYWMH

SEQ ID NO: 24 - protein, 6 aa, Artificial Sequence
  source: /note=“Description of Artificial Sequence Synthetic peptide”
YPGNSD

SEQ ID NO: 25 - protein, 10 aa, Artificial Sequence
  source: /note=“Description of Artificial Sequence Synthetic peptide”
AIYPGNSDTS

SEQ ID NO: 26 - protein, 13 aa, Artificial Sequence
  source: /note=“Description of Artificial Sequence Synthetic peptide”
WIGAIYPGNSDTS

SEQ ID NO: 27 - protein, 11 aa, Artificial Sequence
  source: /note=“Description of Artificial Sequence Synthetic peptide”
TTTTYGYDWFA

SEQ ID NO: 28 - protein, 13 aa, Artificial Sequence
  source: /note=“Description of Artificial Sequence Synthetic peptide”
LYSSNQKNYLAWY

SEQ ID NO: 29 - protein, 10 aa, Artificial Sequence
  source: /note=“Description of Artificial Sequence Synthetic peptide”
LLIYWASTRE

SEQ ID NO: 30 - protein, 7 aa, Artificial Sequence
  source: /note=“Description of Artificial Sequence Synthetic peptide”
HQYLSSL